(19) United States
(12) Patent Application Publication
(10) Pub. No.: US 2024/0108755 A1
(43) Pub. Date: Apr. 4, 2024

(54) COMPOSITIONS AND METHODS FOR TREATING A NEURODEGENERATIVE OR DEVELOPMENTAL DISORDER

(71) Applicant: The Broad Institute, Inc., Cambridge, MA (US)

(72) Inventors: Lindy BARRETT, Cambridge, MA (US); Sara G. SUSCO, Cambridge, MA (US)

(73) Assignee: The Broad Institute, Inc., Cambridge, MA (US)

(21) Appl. No.: 18/472,864

(22) Filed: Sep. 22, 2023

Related U.S. Application Data
(63) Continuation of application No. PCT/US2022/021667, filed on Mar. 24, 2022.
(60) Provisional application No. 63/165,919, filed on Mar. 25, 2021.

(51) Int. Cl. A61K 48/00; A61P 25/00; C07K 14/47; C12N 15/86

(52) U.S. Cl. CPC A61K 48/005; A61P 25/00; C07K 14/47; C12N 15/86; C12N 2750/14143

(57) ABSTRACT

The invention features compositions and methods for treating developmental, neurodevelopmental (e.g., Fragile X syndrome (FXS)) or Down syndrome (DS) or neurodegenerative disorders (e.g., Alzheimer's disease (AD)) by increasing expression of Fragile X Mental Retardation Protein (FMRP) in patients having such disorders.

### CROSS-REFERENCE TO RELATED APPLICATIONS

This application is a continuation under 35 U.S.C. § 111(a) of PCT International Patent Application No. PCT/US2022/021667, filed Mar. 24, 2022, designating the United States and published in English, which claims priority to and the benefit of U.S. Provisional Application No. 63/165,919, filed Mar. 25, 2021, the entire contents of each of which are incorporated by reference herein.

### STATEMENT OF RIGHTS TO INVENTIONS MADE UNDER FEDERALLY SPONSORED RESEARCH

This invention was made with government support under Grant Numbers R01HD101534 and R21MH109761 awarded by the National Institutes of Health. The government has certain rights in the invention.

### SEQUENCE LISTING

The present application contains a Sequence Listing which has been submitted electronically in XML format following conversion from the originally filed TXT format.

The content of the electronic XML Sequence Listing, (Date of creation: Sep. 20, 2023; Size: 22,558 bytes; Name: 167741-037002US-Sequence_Listing.xml), is herein incorporated by reference in its entirety.

### BACKGROUND OF THE INVENTION

Fragile X syndrome (FXS) is the most common inherited cause of intellectual disability and a leading monogenic cause of autism, driven by a trinucleotide repeat expansion in the 5′ UTR of the Fragile X mental retardation 1 (FMR1) gene. This expansion in the FMRI gene leads to epigenetic silencing and loss of the encoded RNA binding protein, Fragile X Mental Retardation Protein (FMRP). While RNA targets of FMRP have been delineated in mouse brain tissue or cells, HEK293T cells, and K562 cells, molecular mechanisms based on these data have failed to translate into effective therapeutic strategies. These targets of FMRP are key targets for therapeutic approaches to FXS, Down syndrome (DS) and Alzheimer's disease (AD), particularly AD associated with DS. Currently, there are no effective treatments for Fragile X syndrome (FXS), Down syndrome (DS) or Alzheimer's disease (AD). Methods of treating such developmental, neurodevelopmental, or neurodegenerative disorders are urgently required.

### SUMMARY OF THE EMBODIMENTS

The present invention features compositions and methods for treating developmental, neurodevelopmental (e.g., Fragile X syndrome (FXS), Down syndrome (DS)), or neurodegenerative diseases or disorders (e.g., Alzheimer's disease (AD)) by increasing expression of Fragile X Mental Retardation Protein (FMRP) in patients having such diseases or disorders.

In an aspect of the invention, a method of modulating the expression, expression level, amount, or activity of a gene as set forth in Table 5, or a polypeptide encoded by such gene, in a cell is provided, in which the method involves contacting the cell with an expression vector encoding a Fragile X mental retardation protein (FMRP) polypeptide or a fragment thereof, thereby modulating the expression, expression level, amount, or activity of the gene, or the encoded polypeptide in the cell. In an embodiment, the expression, expression level, amount, or activity of the gene, or the encoded polypeptide, is increased or enhanced following contact of the cell with FMRP. In an embodiment, the expression, expression level, amount, or activity of the gene, or the encoded polypeptide, is decreased or reduced following contact of the cell with FMRP. In an embodiment, the gene set forth in Table 5 is associated with one or both of Fragile X syndrome (FXS) and Down syndrome (DS). In an embodiment, the gene set forth in Table 5 is associated with both Fragile X syndrome (FXS) and Down syndrome (DS). In an embodiment, the gene set forth in Table 5 is associated with Fragile X syndrome (FXS). In an embodiment, the gene set forth in Table 5 is associated with Down syndrome (DS). In some embodiments, Down syndrome is also associated with another disorder or condition, such as autism, a seizure disorder, or a leukemia. In an embodiment, the expression, expression level, amount, or activity of the gene or the encoded polypeptide is increased or enhanced by at least 5%, 10%, 15%, 20%, 25%, 30%, or greater relative to a normal, non-disease, healthy control cell. In an embodiment, the expression, expression level, amount, or activity of the gene or the encoded polypeptide is decreased or reduced by at least 5%, 10%, 15%, 20%, 25%, 30%, or greater relative to a normal, non-disease, healthy control cell. In an embodiment, the cell is a mammalian cell or is derived from a mammalian subject. In an embodiment, the cell is a human cell or is derived from a human subject. In an embodiment, the cell is in vitro, in vivo, or ex vivo.

In an aspect of the invention, a method of modulating the expression, expression level, amount, or activity of a gene as set forth in Table 5, or the encoded product of the gene, which is associated with a developmental, neurodevelopmental, or neurodegenerative disease or disorder is provided, in which the method involves administering to the subject an effective amount of a Fragile X mental retardation protein (FMRP) polypeptide, or a fragment thereof, or a polynucleotide sequence encoding FMRP, or a fragment thereof, so as to modulate the expression, expression level, amount, or activity of the gene as set forth in Table 5, or the encoded product of the gene. In an embodiment, the expression, expression level, amount, or activity of the gene, or the encoded gene product, is increased or enhanced in the subject following the administration of FMRP to the subject. In an embodiment, the expression, expression level, amount, or activity of the gene, or the encoded gene product, is decreased or reduced in the subject following the administration of FMRP to the subject. In an embodiment, the developmental, neurodevelopmental, or neurodegenerative disease or disorder one or both of Fragile X syndrome (FXS) or Down syndrome (DS). In an embodiment, the gene set forth in Table 5 is associated with both Fragile X syndrome (FXS) and Down syndrome (DS). In an embodiment, the gene set forth in Table 5 is associated with Fragile X syndrome (FXS). In an embodiment, the gene set forth in Table 5 is associated with Down syndrome (DS), or, optionally, a disease or disorder associated with Down syndrome such as autism, a seizure disorder, or a leukemia. In an embodiment, the subject is a mammal. In an embodiment, the subject is a human. In an embodiment, the subject is at risk of having, or has a propensity for having, one or more developmental, neurodevelopmental, or neurodegenerative diseases or disorders. In an embodiment, the developmental, neurodevelopmental or neurodegenerative disease or disorder one or both of Fragile X syndrome (FXS) or Down syndrome (DS). In an embodiment, the polynucleotide sequence encoding FMRP is present in an expression vector. In an embodiment, the expression vector is a lentiviral vector, adenoviral vector, or adeno-associated viral vector (AAV). In an embodiment, the expression, expression level, or amount of the gene as set forth in Table 5, or the encoded product of the gene, is increased or enhanced in a subject by at least 5%, 10%, 15%, 20%, 25%, 30%, or greater following administration of FMRP. In an embodiment, the expression, expression level, or amount of the gene as set forth in Table 5, or the encoded product of the gene, is decreased or reduced in a subject by at least 5%, 10%, 15%, 20%, 25%, 30%, or greater following administration of FMRP.

In another aspect, a method of treating or reducing the severity of a neurodevelopmental or a neurodegenerative disease or disorder, or the symptoms thereof is provided, in which the method comprises modulating the expression, expression level, amount, or activity of one or more genes as set forth in Table 5 in a cell and/or in a subject. In an embodiment of the method, the neurodevelopmental or neurodegenerative disease or disorder is one or both of Fragile X syndrome (FXS) and Down syndrome (DS), or, optionally, a disease or disorder associated with Down syndrome, such as autism, a seizure disorder, or a leukemia.

In another aspect, a method of treating or reducing the severity of one or both of Fragile X syndrome (FXS) and Down syndrome (DS), or the symptoms thereof is provided, in which the method comprises modulating the expression, expression level, amount, or activity of one or more genes as set forth in Table 5 in a cell and/or in a subject.

In another aspect, a method of treating or reducing the severity of one or both of Fragile X syndrome (FXS) and Down syndrome (DS), or the symptoms thereof, in a cell and/or in a subject is provided, in which the method comprises administering or providing to the cell and/or to the subject an effective amount of an agent that modulates the expression, expression level, amount, or activity of one or more genes as set forth in Table 5 in the cell and/or in a subject. In an embodiment of the method, the agent is a polypeptide, a polynucleotide, a drug, or a small molecule compound. In an embodiment, the agent is a Fragile X mental retardation protein (FMRP) polypeptide, or a fragment thereof, or a polynucleotide sequence encoding FMRP, or a fragment thereof.

In an embodiment of the above-delineated methods and/or embodiments thereof, the expression, expression level, amount, or activity of the one or more genes of Table 5 is aberrant or abnormal in the cell and/or the subject relative to the expression, expression level, amount, or activity of the one or more genes in a normal, non-disease control. In an embodiment of the above-delineated methods and/or embodiments thereof, modulation of the expression, expression level, amount, or activity of the one or more genes of Table 5 comprises increasing or enhancing the expression, expression level, amount, or activity of the one or more genes in the cell and/or in the subject to treat or reduce the severity of the neurodevelopmental or neurodegenerative disease or disorder, or one or both of FXS, or DS. In an embodiment of the above-delineated methods and/or embodiments thereof, modulation of the expression, expression level, amount, or activity of the one or more genes of Table 5 comprises decreasing or reducing the expression, expression level, amount, or activity of said one or more genes in the cell and/or in the subject to treat or reduce the severity of the neurodevelopmental or neurodegenerative disease or disorder, or one or both of FXS, or DS. In an embodiment of the above-delineated methods and/or embodiments thereof, the cell is in vivo, in vitro, or ex vivo. In an embodiment of the above-delineated methods and/or embodiments thereof, the subject is a mammal. In an embodiment of the above-delineated methods and/or embodiments thereof, the subject is a human.

In another aspect, a gene panel as set forth in Table 5 is provided, in which the gene panel comprises one or more genes whose expression, expression level, amount, or activity is disrupted or aberrant in one or both of Fragile X syndrome (FXS) or Down syndrome (DS), wherein modulation of the expression, expression level, amount, or activity of the one or more genes in a cell and/or in a subject treats or reduces the severity of one or both of FXS or DS. In an embodiment, modulation of the expression, expression level, amount, or activity of the one or more genes comprises increasing or enhancing the expression, expression level, amount, or activity of the one or more genes in the cell and/or in the subject to treat or reduce the severity of one or both of FXS or DS. In another embodiment, modulation of the expression, expression level, amount, or activity of the one or more genes comprises decreasing or reducing the expression, expression level, amount, or activity of the one or more genes in the cell and/or in the subject to treat or reduce the severity of one or both of FXS or DS. In an embodiment, Fragile X mental retardation protein (FMRP) polypeptide, or a fragment thereof, reverses or ameliorates the disrupted or aberrant expression, expression level, amount, or activity of the one or more genes to treat or reduce the severity of one or both of FXS or DS.

Compositions and articles defined by the invention were isolated or otherwise manufactured in connection with the examples provided below. Other features and advantages of the invention will be apparent from the detailed description, drawings, examples, and tables herein, and from the claims.

### Definitions

Unless defined otherwise, all technical and scientific terms used herein have the meaning commonly understood by a person skilled in the art to which this invention pertains or relates. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); Benjamin Lewin, Genes V, published by Oxford University Press, 1994 (ISBN 0-19-854287-9); Kendrew et al. (eds.); The Encyclopedia of Molecular Biology, published by Blackwell Science Ltd., 1994 (ISBN 0-632-02182-9); Molecular Biology and Biotechnology: a Comprehensive Desk Reference, Robert A. Meyers (ed.), published by VCH Publishers, Inc., 1995 (ISBN 1-56081-569-8); and Hale & Marham, The Harper Collins Dictionary of Biology (1991). As used herein, the following terms have the meanings ascribed to them below, unless specified otherwise.

By “administering” is meant giving, supplying, providing, delivering, or dispensing a composition, agent, therapeutic and the like to a subject, or applying or bringing the composition and the like into contact with the subject. Administering or administration may be accomplished by any of a number of routes, such as, for example, without limitation, topically, orally, subcutaneously, intramuscularly, intraperitoneally, or intravenously (IV).

By “agent” is meant a polypeptide, nucleic acid molecule, or small molecule, as well as fragments of such agents.

By “alteration” or “modulation” is meant a change (an increase, elevation, or enhancement, or a decrease or reduction) in the expression levels or activity of a gene or polypeptide as detected by standard art known methods such as those described herein. As used herein, an alteration includes a 5% change, a 10% change, a 15% change, a 20% change, a 25% change, or greater, such as a 30% change, a 35% change, a 40% change, or a 50% change or greater change in expression level or activity. In embodiments, the change (increase, etc., or decrease, etc.) is relative to a normal, non-disease, healthy control cell, subject, and the like. Accordingly, an increase, etc. or a decrease, etc. may include an at least 5%, 10%, 15%, 20%, 25%, 30%, 35%, 40%, 45%, 50% or greater (including percentage values therebetween) increase, etc., or decrease, etc. in expression, expression level, or activity of a gene (polynucleotide) or the encoded polypeptide product of the gene (polynucleotide).

By “ameliorate” is meant decrease, reduce, delay, suppress, attenuate, diminish, arrest, or stabilize the development or progression of a disease, condition, or pathology.

By “amyloid-beta precursor (APP) polypeptide” is meant a polypeptide having at least about 85% or greater amino acid sequence identity to GenBank Reference Sequence: AAW82435.1 or a fragment thereof that is increased in Alzheimer's disease or Downs Syndrome. An exemplary amino acid sequence of APP is provided below:

>AAW82435.1 amyloid beta (A4) precursor protein (protease nexin-II,
Alzheimer disease) [Homo sapiens]
(SEQ ID NO: 1)
MLPGLALLLLAAWTARALEVPTDGNAGLLAEPQIAMFCGRLNMHMNVQNGKWDSDPSGTKTCIDTKEGILQYCQE
VYPELQITNVVEANQPVTIQNWCKRGRKQCKTHPHFVIPYRCLVGEFVSDALLVPDKCKFLHQERMDVCETHLHW
HTVAKETCSEKSTNLHDYGMLLPCGIDKFRGVEFVCCPLAEESDNVDSADAEEDDSDVWWGGADTDYADGSEDKV
VEVAEEEEVAEVEEEEADDDEDDEDGDEVEEEAEEPYEEATERTTSIATTTTTTTESVEEVVREVCSEQAETGPC
RAMISRWYFDVTEGKCAPFFYGGCGGNRNNFDTEEYCMAVCGSAMSQSLLKTTQEPLARDPVKLPTTAASTPDAV
DKYLETPGDENEHAHFQKAKERLEAKHRERMSQVMREWEEAERQAKNLPKADKKAVIQHFQEKVESLEQEAANER
QQLVETHMARVEAMLNDRRRLALENYITALQAVPPRPRHVFNMLKKYVRAEQKDRQHTLKHFEHVRMVDPKKAAQ
IRSQVMTHLRVIYERMNQSLSLLYNVPAVAEEIQDEVDELLQKEQNYSDDVLANMISEPRISYGNDALMPSLTET
KTTVELLPVNGEFSLDDLQPWHSFGADSVPANTENEVEPVDARPAADRGLTTRPGSGLTNIKTEEISEVKMDAEF
RHDSGYEVHHQKLVFFAEDVGSNKGAIIGLMVGGVVIATVIVITLVMLKKKQYTSIHHGVVEVDAAVTPEERHLS
KMQQNGYENPTYKFFEQMQN

By “amyloid-beta precursor (APP) polynucleotide” is meant a polynucleotide encoding an APP polypeptide. The sequence of an exemplary APP polynucleotide is provided at GenBank Reference Sequence: AK312326.1, which is reproduced below:

>AK312326.1 Homo sapiens cDNA, FLJ92638, Homo sapiens amyloid beta (A4)
precursor protein (proteasenexin-II, Alzheimer disease) (APP), mRNA
(SEQ ID NO: 2)
AGAGCAAGGACGCGGCGGATCCCACTCGCACAGCAGCGCACTCGGTGCCCCGCGCAGGGTCGCAGATGCTGCCCG
GTTTGGCACTGCTCCTGCTGGCCGCCTGGACGGCTCGGGCGCTGGAGGTACCCACTGATGGTAATGCTGGCCTGC
TGGCTGAACCCCAGATTGCCATGTTCTGTGGCAGACTGAACATGCACATGAATGTCCAGAATGGGAAGTGGGATT
CAGATCCATCAGGGACCAAAACCTGCATTGATACCAAGGAAGGCATCCTGCAGTATTGCCAAGAAGTCTACCCTG
AACTGCAGATCACCAATGTGGTAGAAGCCAACCAACCAGTGACCATCCAGAACTGGTGCAAGCGGGGCCGCAAGC
AGTGCAAGACCCATCCCCACTTTGTGATTCCCTACCGCTGCTTAGTTGGTGAGTTTGTAAGTGATGCCCTTCTCG
TTCCTGACAAGTGCAAATTCTTACACCAGGAGAGGATGGATGTTTGCGAAACTCATCTTCACTGGCACACCGTCG
CCAAAGAGACATGCAGTGAGAAGAGTACCAACTTGCATGACTACGGCATGTTGCTGCCCTGCGGAATTGACAAGT
TCCGAGGGGTAGAGTTTGTGTGTTGCCCACTGGCTGAAGAAAGTGACAATGTGGATTCTGCTGATGCGGAGGAGG
ATGACTCGGATGTCTGGTGGGGCGGAGCAGACACAGACTATGCAGATGGGAGTGAAGACAAAGTAGTAGAAGTAG
CAGAGGAGGAAGAAGTGGCTGAGGTGGAAGAAGAAGAAGCCGATGATGACGAGGACGATGAGGATGGTGATGAGG
TAGAGGAAGAGGCTGAGGAACCCTACGAAGAAGCCACAGAGAGAACCACCAGCATTGCCACCACCACCACCACCA
CCACAGAGTCTGTGGAAGAGGTGGTTCGAGAGGTGTGCTCTGAACAAGCCGAGACGGGGCCGTGCCGAGCAATGA
TCTCCCGCTGGTACTTTGATGTGACTGAAGGGAAGTGTGCCCCATTCTTTTACGGCGGATGTGGCGGCAACCGGA
ACAACTTTGACACAGAAGAGTACTGCATGGCCGTGTGTGGCAGCGCCATGTCCCAAAGTTTACTCAAGACTACCC
AGGAACCTCTTGCCCGAGATCCTGTTAAACTCCCTACAACAGCAGCCAGTACCCCTGATGCCGTTGACAAGTATC
TCGAGACACCTGGGGATGAGAATGAACATGCCCATTTCCAGAAAGCCAAAGAGAGGCTTGAGGCCAAGCACCGAG
AGAGAATGTCCCAGGTCATGAGAGAATGGGAAGAGGCAGAACGTCAAGCAAAGAACTTGCCTAAAGCTGATAAGA
AGGCAGTTATCCAGCATTTCCAGGAGAAAGTGGAATCTTTGGAACAGGAAGCAGCCAACGAGAGACAGCAGCTGG
TGGAGACACACATGGCCAGAGTGGAAGCCATGCTCAATGACCGCCGCCGCCTGGCCCTGGAGAACTACATCACCG
CTCTGCAGGCTGTTCCTCCTCGGCCTCGTCACGTGTTCAATATGCTAAAGAAGTATGTCCGCGCAGAACAGAAGG
ACAGACAGCACACCCTAAAGCATTTCGAGCATGTGCGCATGGTGGATCCCAAGAAAGCCGCTCAGATCCGGTCCC
AGGTTATGACACACCTCCGTGTGATTTATGAGCGCATGAATCAGTCTCTCTCCCTGCTCTACAACGTGCCTGCAG
TGGCCGAGGAGATTCAGGATGAAGTTGATGAGCTGCTTCAGAAAGAGCAAAACTATTCAGATGACGTCTTGGCCA
ACATGATTAGTGAACCAAGGATCAGTTACGGAAACGATGCTCTCATGCCATCTTTGACCGAAACGAAAACCACCG
TGGAGCTCCTTCCCGTGAATGGAGAGTTCAGCCTGGACGATCTCCAGCCGTGGCATTCTTTTGGGGCTGACTCTG
TGCCAGCCAACACAGAAAACGAAGTTGAGCCTGTTGATGCCCGCCCTGCTGCCGACCGAGGACTGACCACTCGAC
CAGGTTCTGGGTTGACAAATATCAAGACGGAGGAGATCTCTGAAGTGAAGATGGATGCAGAATTCCGACATGACT
CAGGATATGAAGTTCATCATCAAAAATTGGTGTTCTTTGCAGAAGATGTGGGTTCAAACAAAGGTGCAATCATTG
GACTCATGGTGGGCGGTGTTGTCATAGCGACAGTGATCGTCATCACCTTGGTGATGCTGAAGAAGAAACAGTACA
CATCCATTCATCATGGTGTGGTGGAGGTTGACGCCGCTGTCACCCCAGAGGAGCGCCACCTGTCCAAGATGCAGC
AGAACGGCTACGAAAATCCAACCTACAAGTTCTTTGAGCAGATGCAGAACTAG

The term “effective amount” as used herein refers to the amount of an agent required to ameliorate, reduce, delay, improve, abrogate, abate, diminish, alleviate, or eliminate the symptoms and/or effects of a disease, condition, or pathology relative to an untreated patient, and also relates to a sufficient amount of a pharmacological composition to provide the desired effect. The effective amount of an agent or a composition as used to practice the methods of therapeutic treatment of a disease, condition, or pathology, varies depending upon the manner of administration, the age, body weight, and general health of the subject. Ultimately, the attending physician or veterinarian will decide the appropriate amount and dosage regimen for use according to knowledge and skill in the art. Such amount is referred to as an “effective” amount.

The phrase “therapeutically effective amount” as used herein, e.g., of a polynucleotide encoding FMRP as disclosed herein, means a sufficient amount of the composition to treat a disorder, at a reasonable benefit/risk ratio applicable to any medical treatment. The term “therapeutically effective amount” therefore refers to an amount of an agent or composition as disclosed herein that is sufficient to, for example, effect a therapeutically or prophylactically significant reduction in a symptom or clinical marker associated with a developmental disorder (e.g., autism, Fragile X syndrome, Down syndrome) or a neurodegenerative disorder (e.g., Alzheimer's disease) when administered to a typical subject who has such a disorder. In one embodiment, an effective amount of a polynucleotide encoding FMRP is the amount required to reduce the level of APP.

An effective amount depends on the type of disease to be treated, the severity of the symptoms, the subject being treated, the age and general condition of the subject, the mode of administration and so forth. Thus, it is not possible to specify the exact “effective amount.” However, for any given case, an appropriate “effective amount” can be determined by one of ordinary skill in the art using only routine experimentation. The efficacy of treatment can be judged by an ordinarily skilled practitioner, for example, efficacy can be assessed in animal or in vitro models of a disease. In one embodiment, an effective amount of an FMRP polypeptide or polynucleotide is an amount that leads to a decrease in DYRK1A or APP levels.

Subjects amenable to treatment by the methods as disclosed herein can be identified by any method to diagnose a disease associated with an increase in DYRK1A or APP, such diseases include Fragile X syndrome, Down syndrome, and Alzheimer's disease. Methods of diagnosing these conditions are well known by persons of ordinary skill in the art.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited are not changed by the presence of more than that which is recited, but excludes prior art embodiments.

“Detect” refers to identifying the presence, absence, or amount of an analyte, compound, agent, or substance to be detected or determined. In one embodiment, the analyte is DYRK1A or APP.

By “disease” is meant any condition, disorder, or pathology that damages or interferes with the normal function of a cell, tissue, or organ. Examples of diseases include diseases associated with increased levels of DYRK1A or APP, such as Fragile X syndrome, Down syndrome, and Alzheimer's disease.

By “Dual Specificity Tyrosine Phosphorylation Regulated Kinase 1A (DYRK1A) polypeptide” is meant is meant a polypeptide having at least about 85% or greater amino acid sequence identity to UniProtKB/Swiss-Prot Reference Sequence: Q13627 or a fragment thereof having protein kinase activity. In one embodiment, a DYRK1A polypeptide catalyzes its autophosphorylation on serine/threonine and tyrosine residues. An exemplary amino acid sequence of DYRK1A is provided below:

>sp|Q13627|DYR1A_HUMAN Dual specificity tyrosine-phosphorylation-regulated
kinase 1A OS = Homo sapiens OX = 9606 GN = DYRK1A PE = 1 SV = 2
(SEQ ID NO: 3)
MHTGGETSACKPSSVRLAPSFSFHAAGLQMAGQMPHSHQYSDRRQPNISDQQVSALSYSDQIQQPLTNQVMPDIV
MLQRRMPQTFRDPATAPLRKLSVDLIKTYKHINEVYYAKKKRRHQQGQGDDSSHKKERKVYNDGYDDDNYDYIVK
NGEKWMDRYEIDSLIGKGSFGQVVKAYDRVEQEWVAIKIIKNKKAFLNQAQIEVRLLELMNKHDTEMKYYIVHLK
RHFMFRNHLCLVFEMLSYNLYDLLRNTNFRGVSLNLTRKFAQQMCTALLFLATPELSIIHCDLKPENILLCNPKR
SAIKIVDFGSSCQLGQRIYQYIQSRFYRSPEVLLGMPYDLAIDMWSLGCILVEMHTGEPLFSGANEVDQMNKIVE
VLGIPPAHILDQAPKARKFFEKLPDGTWNLKKTKDGKREYKPPGTRKLHNILGVETGGPGGRRAGESGHTVADYL
KFKDLILRMLDYDPKTRIQPYYALQHSFFKKTADEGTNTSNSVSTSPAMEQSQSSGTTSSTSSSSGGSSGTSNSG
RARSDPTHQHRHSGGHFTAAVQAMDCETHSPQVRQQFPAPLGWSGTEAPTQVTVETHPVQETTFHVAPQQNALHH
HHGNSSHHHHHHHHHHHHHGQQALGNRTRPRVYNSPTNSSSTQDSMEVGHSHHSMTSLSSSTTSSSTSSSSTGNQ
GNQAYQNRPVAANTLDFGQNGAMDVNLTVYSNPRQETGIAGHPTYQFSANTGPAHYMTEGHLTMRQGADREESPM
TGVCVQQSPVASS

By “Dual Specificity Tyrosine Phosphorylation Regulated Kinase 1A (DYRK1A) polynucleotide” is meant a nucleic acid molecule encoding a DYRK1A polypeptide. The sequence of an exemplary DYRK1A polynucleotide is provided at GenBank Reference Sequence: U52373.1, which is reproduced below:

>U52373.1 Human serine/threonine kinase MNB (mnb) mRNA, complete cds
(SEQ ID NO: 4)
GTTATAGTTTTGCCGCTGGACTCTTCCCTCCCTTCCCCCACCCCATCAGGATGATATGAGACTTGAAAGAAGACG
ATGCATACAGGAGGAGAGACTTCAGCATGCAAACCTTCATCTGTTCGGCTTGCACCGTCATTTTCATTCCATGCT
GCTGGCCTTCAGATGGCTGGACAGATGCCCCATTCACATCAGTACAGTGACCGTCGCCAGCCAAACATAAGTGAC
CAACAGGTTTCTGCCTTATCATATTCTGACCAGATTCAGCAACCTCTAACTAACCAGGTGATGCCTGATATTGTC
ATGTTACAGAGGCGGATGCCCCAAACCTTCCGTGACCCAGCAACTGCTCCCCTGAGAAAACTTTCTGTTGACTTG
ATCAAAACATACAAGCATATTAATGAGGTTTACTATGCAAAAAAGAAGCGAAGACACCAACAGGGCCAGGGAGAC
GATTCTAGTCATAAGAAGGAACGGAAGGTTTACAATGATGGTTATGATGATGATAACTATGATTATATTGTAAAA
AACGGAGAAAAGTGGATGGATCGTTACGAAATTGACTCCTTGATAGGCAAAGGTTCCTTTGGACAGGTTGTAAAG
GCATATGATCGTGTGGAGCAAGAATGGGTTGCCATTAAAATAATAAAGAACAAGAAGGCTTTTCTGAATCAAGCA
CAGATAGAAGTGCGACTTCTTGAGCTCATGAACAAACATGACACTGAAATGAAATACTACATAGTGCATTTGAAA
CGCCACTTTATGTTTCGAAACCATCTCTGTTTAGTTTTTGAAATGCTGTCCTACAACCTCTATGACTTGCTGAGA
AACACCAATTTCCGAGGGGTCTCTTTGAACCTAACACGAAAGTTTGCGCAACAGATGTGCACTGCACTGCTTTTC
CTTGCGACTCCAGAACTTAGTATCATTCACTGTGATCTAAAACCTGAAAATATCCTTCTTTGTAACCCCAAACGC
AGTGCAATCAAGATAGTTGACTTTGGCAGTTCTTGTCAGTTGGGGCAGAGGATATACCAGTATATTCAGAGTCGC
TTTTATCGGTCTCCAGAGGTGCTACTGGGAATGCCTTATGACCTTGCCATTGATATGTGGTCCCTCGGGTGTATT
TTGGTTGAAATGCACACTGGAGAACCTCTGTTCAGTGGTGCCAATGAGGTAGATCAGATGAATAAAATAGTGGAA
GTTCTGGGTATTCCACCTGCTCATATTCTTGACCAAGCACCAAAAGCAAGAAAGTTCTTTGAGAAGTTGCCAGAT
GGCACTTGGAACTTAAAGAAGACCAAAGATGGAAAACGGGAGTACAAACCACCAGGAACCCGTAAACTTCATAAC
ATTCTTGGAGTGGAAACAGGAGGACCTGGTGGGCGACGTGCTGGGGAGTCAGGTCATACGGTCGCTGACTACTTG
AAGTTCAAAGACCTCATTTTAAGGATGCTTGATTATGACCCCAAAACTCGAATTCAACCTTATTATGCTCTGCAG
CACAGTTTCTTCAAGAAAACAGCTGATGAAGGTACAAATACAAGTAATAGTGTATCTACAAGCCCCGCCATGGAG
CAGTCTCAGTCTTCGGGCACCACCTCCAGTACATCGTCAAGCTCAGGTGGCTCATCGGGGACAAGCAACAGTGGG
AGAGCCCGGTCGGATCCGACGCACCAGCATCGGCACAGTGGTGGGCACTTCACAGCTGCCGTGCAGGCCATGGAC
TGCGAGACACACAGTCCCCAGGTGCGTCAGCAATTTCCTGCTCCTCTTGGTTGGTCAGGCACTGAAGCTCCTACA
CAGGTCACTGTTGAAACTCATCCTGTTCAAGAAACAACCTTTCATGTAGCCCCTCAACAGAATGCATTGCATCAT
CACCATGGTAACAGTTCCCATCATCACCACCACCACCACCACCATCACCACCACCATGGACAACAAGCCTTGGGT
AACCGGACCAGGCCAAGGGTCTACAATTCTCCAACGAATAGCTCCTCTACCCAAGATTCTATGGAGGTTGGCCAC
AGTCACCACTCCATGACATCCCTGTCTTCCTCAACGACTTCTTCCTCGACATCTTCCTCCTCTACTGGTAACCAA
GGCAATCAGGCCTACCACAATCGCCCAGTGGCTGCTAATACCTTGGACTTTGGACAGAATGGAGCTATGGACGTT
AATTTGACCGTCTACTCCAATCCCCGCCAAGAGACTGGCATAGCTGGACATCCAACATACCAATTTTCTGCTAAT
ACAGGTCCTGCACATTACATGACTGAAGGACATCTGACGATGAGGCAAGGGGCTGATAGAGAAGAGTCCCCCATG
ACAGGAGTTTGTGTGCAACAGAGTCCTGTAGCTAGCTCGTGACTACATTGAAACTTGAGTTTGTTTCTTGTGTGT
TTTTATAGAAGTGGTGTTTTTT

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “fragile X mental retardation protein (FMRP or FMR1) polypeptide” is meant is meant a polypeptide having at least about 85% or greater amino acid sequence identity to UniProtKB/Swiss-Prot Reference Sequence: Q06787 or a fragment thereof having RNA binding activity. An exemplary amino acid sequence of FMRP is provided below:

| (SEQ ID NO: 5) | |
|---|---|
| 1 | meelvvevrg sngafykafv kdvhedsitv afennwqpdr qipfhdvrfp ppvgynkdin |
| 61 | esdevevysr anekepccww lakvrmikge fyvieyaacd atyneivtie rlrsvnpnkp |
| 121 | atkdtfhkik ldvpedlrqm cakeaahkdf kkavgafsvt ydpenyqlvi lsinevtskr |
| 181 | ahmlidmhfr slrtklslim rneeaskqle ssrqlasrfh eqfivredlm glaigthgan |
| 241 | iqqarkvpgv taidldedtc tfhiygedqd avkkarsfle faedviqvpr nlvgkvigkn |
| 301 | gkliqeivdk sgvvrvriea eneknvpqee eimppnslps nnsrvgpnap eekkhldike |
| 361 | nsthfsqpns tkvqrvlvas svvagesqkp elkawqgmvp fvfvgtkdsi anatvlldyh |
| 421 | lnylkevdql rlerlqideq lrqigassrp ppnrtdkeks yvtddgqgmg rgsrpyrnrg |
| 481 | hgrrgpgyts gtnseasnas etesdhrdel sdwslaptee eresflrrgd grrrggggrg |
| 541 | qggrgrgggf kgnddhsrtd nrprnpreak grttdgslqi rvdcnnersv htktlqntss |
| 601 | egsrlrtgkd rnqkkekpds vdgqqplvng vp |

By “fragile X mental retardation protein (FMRP or FMR1) polynucleotide” is meant a polynucleotide encoding a FMRP polypeptide. The sequence of an exemplary FMRP polynucleotide is provided at GenBank Reference Sequence: X69962.1, which is reproduced below:

| (SEQ ID NO: 6) | |
|---|---|
| 1 | acggcgagcg cgggcggcgg cggtgacgga ggcgccgctg ccagggggcg tgcggcagcg |
| 61 | cggcggcggc ggcggcggcg gcggcggcgg aggcggcggc ggcggcggcg gcggcggcgg |
| 121 | aggcggcggc ggcggcggcg gcggcggcgg ctgggcctcg agcgcccgca gcccacctct |
| 181 | cgggggcggg ctcccggcgc tagcagggct gaagagaaga tggaggagct ggtggtggaa |
| 241 | gtgcggggct ccaatggcgc tttctacaag gcatttgtaa aggatgttca tgaagattca |
| 301 | ataacagttg catttgaaaa caactggcag cctgataggc agattccatt tcatgatgtc |
| 361 | agattcccac ctcctgtagg ttataataaa gatataaatg aaagtgatga agttgaggtg |
| 421 | tattccagag caaatgaaaa agagccttgc tgttggtggt tagctaaagt gaggatgata |
| 481 | aagggtgagt tttatgtgat agaatatgca gcatgtgatg caacttacaa tgaaattgtc |
| 541 | acaattgaac gtctaagatc tgttaatccc aacaaacctg ccacaaaaga tactttccat |
| 601 | aagatcaagc tggatgtgcc agaagactta cggcaaatgt gtgccaaaga ggcggcacat |
| 661 | aaggatttta aaaaggcagt tggtgccttt tctgtaactt atgatccaga aaattatcag |
| 721 | cttgtcattt tgtccatcaa tgaagtcacc tcaaagcgag cacatatgct gattgacatg |
| 781 | cactttcgga gtctgcgcac taagttgtct ctgataatga gaaatgaaga agctagtaag |
| 841 | cagctggaga gttcaaggca gcttgcctcg agatttcatg aacagtttat cgtaagagaa |
| 901 | gatctgatgg gtctagctat tggtactcat ggtgctaata ttcagcaagc tagaaaagta |
| 961 | cctggggtca ctgctattga tctagatgaa gatacctgca catttcatat ttatggagag |
| 1021 | gatcaggatg cagtgaaaaa agctagaagc tttctcgaat ttgctgaaga tgtaatacaa |
| 1081 | gttccaagga acttagtagg caaagtaata ggaaaaaatg gaaagctgat tcaggagatt |
| 1141 | gtggacaagt caggagttgt gagggtgagg attgaggctg aaaatgagaa aaatgttcca |
| 1201 | caagaagagg aaattatgcc accaaattcc cttccttcca ataattcaag ggttggacct |
| 1261 | aatgccccag aagaaaaaaa acatttagat ataaaggaaa acagcaccca tttttctcaa |
| 1321 | cctaacagta caaaagtcca gagggtgtta gtggcttcat cagttgtagc aggggaatcc |
| 1381 | cagaaacctg aactcaaggc ttggcagggt atggtaccat ttgtttttgt gggaacaaag |
| 1441 | gacagcatcg ctaatgccac tgttcttttg gattatcacc tgaactattt aaaggaagta |
| 1501 | gaccagttgc gtttggagag attacaaatt gatgagcagt tgcgacagat tggagctagt |
| 1561 | tctagaccac caccaaatcg tacagataag gaaaaaagct atgtgactga tgatggtcaa |
| 1621 | ggaatgggtc gaggtagtag accttacaga aatagggggc acggcagacg cggtcctgga |
| 1681 | tatacttcag gaactaattc tgaagcatca aatgcttctg aaacagaatc tgaccacaga |
| 1741 | gacgaactca gtgattggtc attagctcca acagaggaag agagggagag cttcctgcgc |
| 1801 | agaggagacg gacggcggcg tggaggggga ggaagaggac aaggaggaag aggacgtgga |
| 1861 | ggaggcttca aaggaaacga cgatcactcc cgaacagata atcgtccacg taatccaaga |
| 1921 | gaggctaaag gaagaacaac agatggatcc cttcagatca gagttgactg caataatgaa |
| 1981 | aggagtgtcc acactaaaac attacagaat acctccagtg aaggtagtcg gctgcgcacg |
| 2041 | ggtaaagatc gtaaccagaa gaaagagaag ccagacagcg tggatggtca gcaaccactc |
| 2101 | gtgaatggag taccctaaac tgcataattc tgaagttata tttcctatac catttccgta |
| 2161 | attcttattc catattagaa aactttgtta ggccaaagac aaatagtagg caagatggca |
| 2221 | cagggcatga aatgaacaca aattatgcta agaatttttt attttttggt attggccata |
| 2281 | agcaacaatt ttcagatttg cacaaaaaga taccttaaaa tttgaaacat tgcttttaaa |
| 2341 | actacttagc acttcagggc agattttagt tttattttct aaagtactga gcagtgatat |
| 2401 | tctttgttaa tttggaccat tttcctgcat tgggtgatca ttcaccagta cattctcagt |
| 2461 | ttttcttaat atatagcatt tatggtaatc atattagact tctgttttca atctcgtata |
| 2521 | gaagtcttca tgaaatgcta tgtcatttca tgtcctgtgt cagtttatgt tttggtccac |
| 2581 | ttttccagta ttttagtgga ccctgaaatg tgtgtgatgt gacatttgtc attttcatta |
| 2641 | gcaaaaaaag ttgtatgatc tgtgcctttt ttatatcttg gcaggtagga atattatatt |
| 2701 | tggatgcaga gttcagggaa gataagttgg aaacactaaa tgttaaagat gtagcaaacc |
| 2761 | ctgtcaaaca ttagtacttt atagaagaat gcatgctttc catatttttt tccttacata |
| 2821 | aacatcaggt taggcagtat aaagaatagg acttgttttt gtttttgttt tgttgcactg |
| 2881 | aagtttgata aatagtgtta ttgagagaga tgtgtaattt ttctgtatag acaggagaag |
| 2941 | aaagaactat cttcatctga gagaggctaa aatgttttca gctaggaaca aatcttcctg |
| 3001 | gtcgaaagtt agtaggatat gcctgctctt tggcctgatg accaatttta acttagagct |
| 3061 | ttttttttta attttgtctg ccccaagttt tgtgaaattt ttcatatttt aatttcaagc |
| 3121 | ttattttgga gagataggaa ggtcatttcc atgtatgcat aataatcctg caaagtacag |
| 3181 | gtactttgtc taagaaacat tggaagcagg ttaaatgttt tgtaaacttt gaaatatatg |
| 3241 | gtctaatgtt taagcagaat tggaaaagac taagatcggt taacaaataa caactttttt |
| 3301 | ttcttttttt cttttgtttt ttgaagtgtt ggggtttggt tttgtttttt gagtcttttt |
| 3361 | tttttaagtg aaatttattg aggaaaaata tgtgaaggac cttcactcta agatgttata |
| 3421 | tttttcttaa aaagtaactc ctagtagggg taccactgaa tctgtacaga gccgtaaaaa |
| 3481 | ctgaagttct gcctctgatg tattttgtga gtttgtttct ttgaattttc attttacagt |
| 3541 | tacttttcct tgcatacaaa caagcatata aaatggcaac aaactgcaca tgatttcaca |
| 3601 | aatattaaaa agtcttttaa aaagtattgc caaacattaa tgttgatttc tagttattta |
| 3661 | ttctgggaat gtatagtatt tgaaaacaga aattggtacc ttgcacacat catctgtaag |
| 3721 | ctgtttggtt ttaaaatact gtagataatt aaccaaggta gaatgacctt gtaatgtaac |
| 3781 | tgctcttggg caatattctc tgtacatatt agcgacaaca gattggattt tatgttgaca |
| 3841 | tttgtttggt tatagtgcaa tatattttgt atgcaagcag tttcaataaa gtttgatctt |
| 3901 | cctctgctaa attgatgttg atgcaatcct tacaaatgat tgcttttaaa attttaagct |
| 3961 | aggaaaagaa atctatagaa agtgttctgt tacaaaatgt aactgttacc attggaaatt |
| 4021 | tcacgtcata ggaagttagc ctttatctac ccaactttca agaaggttct ttaataaagc |
| 4081 | gaaaactcaa ccaaatggta cttttccaca gtgtaccatt aaaatatgca ctagtctctt |
| 4141 | tttacaaggc tgtattcagc aagggcctaa cttgcttaaa gtgtaattac taacttctaa |
| 4201 | aactgtactt tgattcacat gttttcaaat ggagttggag ttcattcata ttacaatatt |
| 4261 | tgtgtgctaa acgtgtatgt ttttcagttc aaagtcatga tgtttttaaa atcttattaa |
| 4321 | agtttcaaaa atctgaagat tgtttatcta gatgtaaatt tt |

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified. The term “isolated” also embraces recombinant nucleic acids or proteins, as well as chemically synthesized nucleic acids or peptides.

By “marker” is meant any protein or polynucleotide that has an alteration in expression level or activity that is associated with a disease, condition, pathology, or disorder. In one embodiment, a marker is APP or DYRK1A.

As used herein, “obtaining” as in “obtaining an agent” includes synthesizing, isolating, purchasing, or otherwise acquiring the agent.

The term “pharmaceutically acceptable vehicle” refers to conventional carriers (vehicles), excipients, or diluents that are physiologically and pharmaceutically acceptable for use, particularly in mammalian, e.g., human, subjects. Such pharmaceutically acceptable vehicles are known to the skilled practitioner in the pertinent art and can be readily found in Remington's Pharmaceutical Sciences, by E. W. Martin, Mack Publishing Co., Easton, Pa., 15th Edition (1975) and its updated editions, which describes compositions and formulations suitable for pharmaceutical delivery of one or more therapeutic or immunogenic compositions, such as one or more vaccines, and additional pharmaceutical agents. In general, the nature of a pharmaceutically acceptable carrier depends on the particular mode of administration being employed. For instance, parenteral formulations usually comprise injectable fluids/liquids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers may include, for example, pharmaceutical grades of mannitol, lactose, starch, or magnesium stearate, which typically stabilize and/or increase the half-life of a composition or drug. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

By “reduces” is meant a negative alteration or a reduction of at least 5%, 10%, 15%, 20%, 25%, 30%, 40%, 50%, 75%, 80%, 85%, 90%, 95%, 98%, or 100%. In one embodiment, expression of heterologous FMRP in a cell desirably reduces levels of APP or DYRK1A.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. A reference sequence includes any of the sequences provided herein.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule.

By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, at least 65%, at least 70%, at least 75%, at least 80%, at least 85%, at least 90%, at least 95%, or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a non-human primate, a rodent, a bovine, equine, canine, ovine, or feline mammal.

Ranges provided herein are understood to be shorthand for all of the values within the range, inclusive of the first and last stated values. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 32, 33, 34, 35, 36, 37, 38, 39, 40, 41 42, 43, 44, 45, 46, 47, 48, 49, 50 or greater, consecutively, such as to 100 or greater.

The term “tissue” refers to a group or layer of similarly specialized cells which together perform certain special functions.

The terms “treat,” “treating,” “treatment,” and the like refer to reducing, diminishing, decreasing, delaying, abrogating, ameliorating, or eliminating, a disease, condition, disorder, or pathology, and/or symptoms associated therewith. While not intending to be limiting, “treating” typically relates to a therapeutic intervention that occurs after a disease, condition, disorder, or pathology, and/or symptoms associated therewith, have begun to develop to reduce the severity of the disease, etc., and the associated signs and symptoms. It will be appreciated that, although not precluded, treating a disorder or condition does not require that the disease, condition, disorder, pathology, or the symptoms associated therewith, be completely eliminated. In some embodiments, a disease or disorder, such as Fragile X syndrome, Down syndrome, or Alzheimer's disease, is treated when levels of APP are reduced.

The terms “prevent,” “preventing,” “prevention,” “prophylactic treatment” and the like, refer to inhibiting or blocking a disease state, or the full development of a disease in a subject, or reducing the probability of developing a disease, disorder or condition in a subject, who does not have, but is at risk of developing, or is susceptible to developing, a disease, disorder, or condition.

Unless specifically stated or obvious from context, as used herein, the term “or” is understood to be inclusive. Unless specifically stated or obvious from context, as used herein, the terms “a,” “an,” and “the” are understood to be singular or plural. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description.

Unless specifically stated or obvious from context, as used herein, the term “about” is understood as within a range of normal tolerance in the art, for example within 2 standard deviations of the mean. About may be understood as being within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof

Any compositions or methods provided herein can be combined with one or more of any of the other compositions and methods provided herein.

### BRIEF DESCRIPTION OF THE DRAWINGS

FIGS. 1A-1K demonstrate FMRP RNA targets in human pluripotent stem cells (hPSCs) and neurons defined by enhanced crosslinking and immunoprecipitation followed by high-throughput sequencing (eCLIP-seq). FIG. 1A is a schematic of FMRP eCLIP-seq including cell types and genotypes generated, cross-linking and immunoprecipitation with different FMRP antibodies from each cell type/genotype, enrichment of reads in the immunoprecipitation (IP) condition over the size-matched input (SMInput) and the strategy used for FMR1y/− peak subtraction from each corresponding FMR1y/+ dataset. FIG. 1B is a graph depicting FMRP peak locations for each of the indicated eCLIP-seq datasets including coding sequence (CDS), intron, 5′ UTR, 3′ UTR, and other sequences (i.e., intergenic, noncoding exon, stop codon). FIG. 1C is a graph showing the percentage of individual gene targets from each eCLIP-seq dataset with FMRP binding events in introns. FIG. 1D is a graph showing the results of a gene ontology (GO) analysis of hematopoietic stem cells (hPCSs). Select GO terms for genes identified from significant peaks in hPSCs are shown. Fold enrichment for each GO term is plotted on the x-axis, with the Bonferroni corrected p-value annotated in each bar. FIG. 1E is a graph showing the results of a gene ontology (GO) analysis of neurons. Select GO terms for all genes identified from significant peaks in neurons (from RN016P and ab17722 datasets) are shown. Fold enrichment for each GO term is plotted on the x-axis, with the Bonferroni corrected p-value annotated in each bar. FIG. 1F is a Venn diagram demonstrating the overlap (shown as the number of genes) between all unique FMRP targets identified in neurons (left circle) and FMRP targets in hPSCs (right circle), p=4.3×10⁻²⁸. FIG. 1G is a Venn diagram demonstrating the overlap (shown as number of genes) between all unique FMRP targets identified in neurons (left circle) and the human homologs of mouse FMRP targets previously identified in mouse brain by Damell et al., 2011 (right circle), p=3.92×10⁻¹³⁸. Significance was determined by hypergeometric test for over enrichment. FIG. 1H is a pie chart showing the percent of all unique FMRP neuron targets that were also detectably expressed in fetal brain (95.2%) and not detectably expressed in human fetal brain (4.8%) based on comparison with the Allen BrainSpan Atlas. FIGS. 1I-1K illustrate the results of a K-mer analysis and analysis of base pairing probabilities from FMRP eCLIP-seq data do not implicate specific motif or structural feature driving target recognition. FIG. 1I is a dot plot generated by a K-mer analysis for all significant peaks from FMRP neuron (ab17722) eCLIP-seq (x-axis) versus k-mer analysis for intron peaks from FMRP neuron (ab17722) eCLIP-seq (y-axis). Values are z-scores and labels note the six sequences found most frequently across the intron peaks. FIG. 1J is a graph showing base pairing probabilities, as a measure of RNA structuredness, calculated in FMRP neuron (ab17722) intron binding sites of length 50 or less (middle two data sets) and flanking sites (data sets flanking the middle two data sets) for comparison. FIG. 1K is a graph showing base pairing probabilities, as a measure of RNA structuredness, calculated in FMRP neuron (ab17722) intron binding sites (middle two data sets) and flanking sites (data sets flanking the middle two data sets) for comparison for FMRP binding sites of length 200 or less. For FIGS. 1J and 1K, base pairing probability values in flanking or binding regions were averaged across each FMRP binding site. No differences were observed between FMRP binding sites and their flanking regions, indicating a lack of support for a highly structured region that specifically recruits FMRP binding.

FIGS. 2A-2F demonstrate that a majority of FMRP binding events in neurons are mediated by the key co-factor Fragile X mental retardation syndrome related 1 protein (FXR1P). FIG. 2A is an image of an FMRP IP-Western blot analysis in neurons showing association with FXR1P. Samples were blotted for FXR1P after immunoprecipitating FMRP from FMR1y/+ neurons (left), IgG from FMR1y/+ neurons (center), and FMRP from FMR1y/− neurons (right). Input, supernatant, and wash lanes are also shown for each immunoprecipitation. FXR1P only appears when immunoprecipitated with FMRP from FMR1y/+ neurons. FIG. 2B is a schematic of FXR1+/+ and FXR1−/− isogenic cell line generation using CRISPR-Cas9. FIG. 2C is an image of a Western blot validation showing expected FXR1P loss in FXR1−/− hPSCs. FIG. 2D comprises a schematic of FMRP eCLIP-seq including cell type and genotypes generated, cross-linking and immunoprecipitation with the FMRP antibody ab17722 (Abcam), enrichment of reads in the IP condition over the SMlnput and the strategy used for FMR1y/− peak subtraction. FIG. 2E is a Venn diagram illustrating the overlap (shown as number of genes) between FMRP targets in FXR1−/− neurons (left) and FMRP targets in FXR1+/+ neurons from FIGS. 1F and 1G (right). FIG. 2F is a graph showing peak locations for the FMRP eCLIP-seq dataset generated in FXR1−/− neurons (left) compared to FXR1+/+ neurons (right) from FIG. 1B, including CDS, intron, 5′ UTR, 3′ UTR, and other sequences (i.e., intergenic, noncoding exon, stop codon).

FIGS. 3A-3J demonstrate that FMRP targets are enriched for key neurodevelopmental disease associated genes. FIG. 3A comprises pie charts showing the percentage of genes implicated in developmental disorders, autism, and Down syndrome that are detectably expressed in human neurons or not detectably expressed in human neurons. FIG. 3B comprises pie charts showing percent of expressed genes implicated in developmental disorders, autism, and Down syndrome from FIG. 3A that are FMRP targets in human neurons or not FMRP targets in human neurons using all unique FMRP targets identified in neurons. FIG. 3C is a graph for each disease dataset showing the fold enrichment for FMRP targets plotted on the y-axis, with the p-value annotated in each bar (Developmental Disorders p=5.11×10⁻⁹; Autism p=4.25×10⁻¹¹; Down syndrome p=2.79×10⁻¹⁵). p-values were calculated by hypergeometric test for over enrichment with Bonferroni correction. FIGS. 3D-3F illustrate that FMRP targets are enriched for neurodevelopmental disease associated genes in hPSCs. FIG. 3D comprises pie charts show percent of genes implicated in developmental disorders, autism and Down syndrome that are detectably expressed in hPSCs or not detectably expressed in hPSCs. FIG. 3E comprises pie charts showing percent of expressed genes implicated in developmental disorders, autism and Down syndrome from FIG. 3D that are FMRP targets in hPSCs or not FMRP targets in hPSCs. FIG. 3F is a graph for each disease dataset showing the fold enrichment for FMRP targets plotted on the y-axis, with the p-value annotated in each bar (Developmental Disorders p=1.22×10⁻⁹; Autism p=1.04×10⁻⁶; Down syndrome p=6.70×10⁻⁴). p-values were calculated by hypergeometric test for over enrichment with Bonferroni correction. FIG. 3G is an ideogram of HSA21 showing all unique FMRP RNA targets identified in neurons only, hPSCs only, or both neurons and hPSCs. Scale bar=10 Mb. FIGS. 3H and 3I demonstrate that cells used for FMRP eCLIP-seq are karyotypically normal. FIG. 3H is a graph illustrating a B allele frequency analysis from SNP array data that demonstrates no chromosomal abnormalities in cells used for FMR1y/+ eCLIP-seq. The plot shows a two-dimensional histogram with 20 vertical bins and one horizontal bin for each megabase. Grey boxes are centromeres. FIG. 3I is an image of a cytogenic analysis of G-banded metaphase cells that also shows a normal 46, XY karyotype for cells used in FMR1y/+ eCLIP-seq experiments. FIG. 3J is a heatmap of relative enrichment of RBP binding sites on chromosomes 1-22, X, and Y, which shows that RNA binding proteins (RBPs) do not show general preference for transcripts from HSA21 in K562 ENCODE dataset. This includes 120 RBPs with eCLIP data from K562 cells collected as part of the ENCODE Consortium Project. Relative enrichment for each RBP was calculated as the number of replicable binding sites occurring on a given chromosome, adjusted by the transcribed and mappable space of the chromosome and the total number of binding sites detected for the given RBP.

FIGS. 4A-4M illustrate that FXS and DS converge on shared transcriptional perturbations. FIG. 4A is a schematic of cells used for RNA-seq including isogenic FMR1y/+ and FMR1y/− hPSCs and neurons, as well as isogenic trisomy 21 (T21; DS iPSC A) and euploid control hPSCs and neurons. FIG. 4B is a volcano plot for differentially expressed genes in FXS hPSCs. FIG. 4C is a volcano plot for differentially expressed genes in FXS neurons. FIG. 4D is a volcano plot for differentially expressed genes in DS hPSCs. FIG. 4E is a volcano plot for differentially expressed genes in DS neurons. Shaded boxes indicate genes that have a p-value ≤0.05. Log2 fold change is shown on the x-axis and −log10 adjusted p-value is shown on the y-axis. Positive fold change indicates increased expression in FMR1y/− or DS cells, respectively. FIG. 4F is a volcano plot of HSA21-encoded transcripts from DS neurons. Log2 fold change is shown on the x-axis, with the −log10 of the adjusted p-value shown on the y-axis for all transcripts detected from HSA21 in neurons. Positive fold change reflects an increase in DS neurons relative to euploid control neurons. Transcripts that reach significance of p≤0.05 are shown in grey. Five biological replicates were sequenced for each genotype. FIG. 4G is a volcano plot of HSA21-encoded transcripts from DS hPSCs. Log2 fold change is shown on the x-axis, with the −log10 of the adjusted p-value shown on the y-axis for all transcripts detected from HSA21 in hPSCs. Positive fold change reflects an increase in DS hPSCs relative to euploid hPSCs. Transcripts that reach significance of p≤0.05 are shown in grey. FIG. 4H comprises box plots as examples of expression patterns for two HSA21-encoded transcripts in hPSCs, Dual Specificity Tyrosine Phosphorylation Regulated Kinase 1A (DYRK1A) (p=2.85×10⁻¹⁸) and Amyloid Beta Precursor Protein (APP) (p=1.88×10⁻⁵³). Transcript per million (TPM) values are shown for five biological replicates per cell type/genotype. FIG. 4I is a graph showing the percentage of eCLIP-seq targets that were detected as significantly differentially expressed by RNA-seq (darker shading; p=8.28×10⁻⁵ for hPSCs and p=0.000232 for neurons). The number of eCLIP-seq targets detected as differentially expressed by RNA-seq is indicated for each cell type. FIG. 4J provides box plots as examples of expression patterns for individual FMRP eCLIP-seq targets from isogenic FMR1y/+ and FMR1y/− datasets including NCAM2 (p=0.000154), CBS, DYRK1A, and PCP4. Transcript per million (TPM) values are shown for five biological replicates per cell type/genotype. FIG. 4K is a graph showing the percentages of significant differentially expressed genes (DEGs) detected in the isogenic DS model that were also shared with the isogenic FXS model (darker shading; p=3.19×10⁻³³ for hPSC overlap and p=0.00495 for neuron overlap). FIG. 4L is a graph showing the percentage of significant DEGs detected in the FXS models that were also shared with the DS models (darker shading; p=3.19×10⁻³³ for hPSC overlap and p=0.00495 for neuron overlap). FIG. 4M provides box plots showing the expression patterns for individual genes coordinately regulated at the transcript level in FXS and DS models including NOVA2 (FXS comparison p=0.00433; DS comparison p=0.000802), TAC1 (FXS comparison p=1.08×10⁻⁴⁹; DS comparison p=3.52×10⁻²⁶), NIP2A (FXS comparison p=8.05×10⁻⁹; DS comparison p=2.92×10⁻⁵), SOX11 (FXS comparison p=0.0253; DS comparison p=0.0058), and TUSC3 (FXS comparison p=0.03; DS comparison p=0.00149). TPM values are shown for 5 biological replicates per cell type/genotype. For FIGS. 4G, 4H, 4J, and 4M, significance was calculated by Benjamini-Hochberg adjusted Wald test. For FIGS. 4I, 4, J, and 4K, significance was calculated by hypergeometric test for over enrichment. Significance is indicated by *p≤0.05, **p≤0.005 and ***p≤0.0005 relative to the indicated control.

FIGS. 5A-5P show that FXS and DS converge on shared proteomic perturbations. FIG. 5A is a karyotype showing the G-banding of the DS iPSC lines and euploid iPSC line acquired from Weick et al., 2013, that confirms trisomy 21 for DS iPSC A. FIG. 5B is a karyotype showing the G-banding of the DS iPSC lines and euploid iPSC line acquired from Weick et al., 2013, that confirms trisomy 21 for DS iPSC B. FIG. 5C is a normal karyotype for the euploid iPSC acquired from Weick et al., 2013. FIG. 5D is an image of a Western blot for FMRP in neurons from FMR1y/+ and FMR1y/− isogenic cell lines, euploid iPSC and DS iPSC A isogenic cell lines, plus additional FXS patient iPSC lines (FXS iPSC A, FXS iPSC B, FXS iPSC C) and an additional DS patient iPSC line (DS iPSC B). The blot for the GAPDH loading control is shown below FMRP. As expected, neurons generated from the FXS patient lines and FMR1y/− line lack FMRP expression. FIGS. 5E-5J present bar graphs showing the quantification of Western blots performed in triplicate demonstrating individual cell line results including three control cell lines (FMR1^{y/+} hESCs, Control iPSC A and control iPSC B), four FXS cell lines (CRISPR-engineered FMR1^{y/-} hESCs plus FXS patient iPSC lines A-C) and two DS cell lines (DS iPSC A and B). Pooled results are shown. Proteins queried include the HSA21-encoded FMRP targets CBS (FIG. 5E), NCAM2 (FIG. 5F) PCP4 (FIG. 5G) DYRK1A (FIG. 5H) APP (FIG. 5I) and BACE2 (FIG. 5J). Error bars show SEM and significance between each control and disease sample as calculated by unpaired two-tailed t-test. Significance is indicated by *p≤0.05, **p≤0.005 and ***p≤0.0005 relative to controls. FIG. 5E presents a graph showing the quantification of a Western blot that queried the HSA21-encoded FMRP target CBS in hPSCs. FIG. 5F presents a graph showing the quantification of a Western blot that queried the HSA21-encoded FMRP target NCAM2 in neurons. FIG. 5G presents a graph showing the quantification of a Western blot that queried the HSA21-encoded FMRP target PCP4 in neurons. FIG. 5H presents a graph showing the quantification of a Western blot that queried the HSA21-encoded FMRP target DYRK1A in neurons. FIG. 5I presents a graph showing the quantification of a Western blot that queried the HSA21-encoded FMRP target APP in hPSCs. FIG. 5J presents a graph showing the quantification of a Western blot that queried the HSA21-encoded FMRP target BACE2 in hPSCs. Cell types used for western blots were determined based on where FMRP bound targets in eCLIP-seq data. In cases where FMRP bound a given target in both hPSCs and neurons, a single cell type was selected for analysis. FIG. 5K comprises gene diagrams showing location of corresponding eCLIP-seq peaks for genes queried by western blot analysis. Arrows indicate eCLIP-seq peaks in CDS regions, introns and a non-coding exon. The bar above each diagram is scaled to 50 kb. FIG. 5L is a schematic of a CRISPRa experiment. TRE-dCas9-VPR-eGFP was stably integrated into the AAVS1 safe-harbor locus of the DS iPSC A cell line along with three FMR1 activating gRNAs introduced with a multiplexed piggyBac integration strategy. FIG. 5M is a time-course of doxycycline (dox) treatment and sample collection. FIG. 5N comprises a graph quantifying the data observed in the image of a FMRP Western blot performed using an isogenic euploid control iPSC line plus the DS-CRISPRa cell line with indicated treatment conditions. FIG. 5O comprises a graph quantifying the data observed in the image of a DYRK1A Western blot performed using an isogenic euploid control iPSC line plus the DS-CRISPRa cell line with indicated treatment conditions. FIG. 5P comprises a graph quantifying the data observed in the image of an APP Western blot performed using an isogenic euploid control iPSC line plus the DS-CRISPRa cell line with indicated treatment conditions. For FIGS. 5N, 5O, and 5P, the no dox condition is compared with the 48 hr dox, 120 hr dox and 120 hr off dox conditions (all DS CRISPRa cell line) and the isogenic euploid control is used as a reference point for non-DS expression levels. All western blots were performed in triplicate. Error bars show SEM and significance was calculated by unpaired two-tailed t-test. Significance is indicated by *p≤0.05, **p≤0.005 relative to the indicated control.

FIGS. 6A-6D provide summary schematics. FIG. 6A is an illustration of the synthesis of predicted FMRP functions from the literature. FMRP has been implicated in multiple aspects of transcriptional and post-transcriptional processing, which means that individual transcript targets could be impacted at different or multiple stages of processing. FMRP is schematized as a blue oval, gray ovals refer to other proteins, DNA is represented by solid lines and RNA is represented by dashed lines. m6A modifications are shown as red triangles. Previously reported binding motifs are shown in brown. FIG. 6B comprises illustrations of a large subset of HSA21-encoded transcripts are bound by FMRP. Under normal conditions, FMRP is thought to play an inhibitory role in RNA processing and there are two copies of HSA21. FIG. 6C is an illustration showing that in the context of FXS, loss of FMRP could lead to increased protein expression through multiple mechanisms of transcript regulation shown in FIG. 6A. Similarly, in the context of DS, an extra copy of HSA21 could lead to increased protein expression. FIG. 6D is an illustration showing that increasing FMRP expression in the context of DS is sufficient to reduce DYRK1A and APP protein expression levels.

### DETAILED DESCRIPTION OF THE EMBODIMENTS

The present invention features compositions and methods for treating developmental, neurodevelopmental (e.g., Fragile X syndrome (FXS) or Down syndrome (DS)), or neurodegenerative diseases or disorders (e.g., Alzheimer's disease (AD)) by increasing expression of Fragile X Mental Retardation Protein (FMRP) in patients having such diseases or disorders.

The invention is based, at least in part, on the discovery that increasing FMRP levels is sufficient to significantly reduce protein expression levels of APP and DYRK1A, excess levels of which are associated with neurodevelopmental (e.g., Fragile X syndrome (FXS), Downs Syndrome) and/or neurodegenerative diseases or disorders (e.g., Alzheimer's disease (AD)) by increasing expression of Fragile X Mental Retardation Protein (FMRP) in patients having such diseases or disorders.

The invention is further based, in part, on the discovery that the increased or decreased expression, expression level, amount, or activity (e.g., aberrant activity) of a gene as set forth in Table 5, shown in the Appendix provided infra, or a polypeptide encoded by such gene, in a cell and/or in a subject, was determined to be associated with one or more neurodevelopmental or neurodegenerative diseases or disorders, such as FXS and/or DS, and/or the symptoms thereof.

The invention relates to a method of modulating the expression, expression level, amount, or activity of a gene as set forth in Table 5, or a polypeptide encoded by such gene, in a cell, in which the method involves contacting the cell with an expression vector encoding a Fragile X mental retardation protein (FMRP) polypeptide or a fragment thereof, thereby modulating the expression, expression level, amount, or activity of the gene, or the encoded polypeptide in the cell. In an embodiment, the expression, expression level, amount, or activity of the gene, or the encoded polypeptide, is increased or enhanced following contact of the cell with FMRP. In an embodiment, the expression, expression level, amount, or activity of the gene, or the encoded polypeptide, is decreased or reduced following contact of the cell with FMRP. In an embodiment, the gene set forth in Table 5 is associated with one or both of Fragile X syndrome (FXS) and Down syndrome (DS). In an embodiment, the gene set forth in Table 5 is associated with both Fragile X syndrome (FXS) and Down syndrome (DS). In an embodiment, the gene set forth in Table 5 is associated with Fragile X syndrome (FXS). In an embodiment, the gene set forth in Table 5 is associated with Down syndrome (DS). In some embodiments, Down syndrome is also associated with another disorder or condition, such as autism, a seizure disorder, or a leukemia. In an embodiment, the expression, expression level, amount, or activity of the gene or its encoded polypeptide is increased or enhanced by at least 5%, 10%, 15%, 20%, 25%, 30%, or greater relative to a normal, non-disease, healthy control cell. In an embodiment, the expression, expression level, amount, or activity of the gene or the encoded polypeptide is decreased or reduced by at least 5%, 10%, 15%, 20%, 25%, 30%, or greater relative to a normal, non-disease, healthy control cell. In an embodiment, the cell is a mammalian cell or is derived from a mammalian subject. In an embodiment, the cell is a human cell or is derived from a human subject. In an embodiment, the cell is in vitro, in vivo, or ex vivo.

The invention further relates to a method of modulating the expression, expression level, amount, or activity of a gene as set forth in Table 5, or the encoded product of the gene, which is associated with a neurodevelopmental, or neurodegenerative disease or disorder, in which the method involves administering to the subject an effective amount of a Fragile X mental retardation protein (FMRP) polypeptide, or a fragment thereof, or a polynucleotide sequence encoding FMRP, or a fragment thereof, so as to modulate the expression, expression level, amount, or activity of the gene as set forth in Table 5, or the encoded product of the gene. In an embodiment, the expression, expression level, amount, or activity of the gene, or the encoded gene product, is increased or enhanced in the subject following the administration of FMRP to the subject. In an embodiment, the expression, expression level, amount, or activity of the gene, or the encoded gene product, is decreased or reduced in the subject following the administration of FMRP to the subject. In an embodiment, the neurodevelopmental, or neurodegenerative disease or disorder is one or both of Fragile X syndrome (FXS) or Down syndrome (DS). In an embodiment, the gene set forth in Table 5 is associated with both Fragile X syndrome (FXS) and Down syndrome (DS). In an embodiment, the gene set forth in Table 5 is associated with Fragile X syndrome (FXS). In an embodiment, the gene set forth in Table 5 is associated with Down syndrome (DS), or, optionally, a disease or disorder associated with Down syndrome such as autism, a seizure disorder, or a leukemia. In an embodiment, the subject is a mammal. In an embodiment, the subject is a human. In an embodiment, the subject is at risk of having, or has a propensity for having, one or more neurodevelopmental, or neurodegenerative diseases or disorders. In an embodiment, the neurodevelopmental or neurodegenerative disease or disorder one or both of Fragile X syndrome (FXS) or Down syndrome (DS). In an embodiment, the polynucleotide sequence encoding FMRP is present in an expression vector. In an embodiment, the expression vector is a lentiviral vector, adenoviral vector, or adeno-associated viral vector (AAV). In an embodiment, the expression, expression level, or amount of the gene as set forth in Table 5, or the encoded product of the gene, is increased or enhanced in a subject by at least 5%, 10%, 15%, 20%, 25%, 30%, or greater following administration of FMRP. In an embodiment, the expression, expression level, or amount of the gene as set forth in Table 5, or the encoded product of the gene, is decreased or reduced in a subject by at least 5%, 10%, 15%, 20%, 25%, 30%, or greater following administration of FMRP.

The invention also relates to a method of treating or reducing the severity of a neurodevelopmental or a neurodegenerative disease or disorder, or the symptoms thereof, in which the method comprises modulating the expression, expression level, amount, or activity of one or more genes as set forth in Table 5 in a cell and/or in a subject. In an embodiment of the method, the neurodevelopmental or neurodegenerative disease or disorder is one or both of Fragile X syndrome (FXS) and Down syndrome (DS), or, optionally, a disease or disorder associated with Down syndrome, such as autism, a seizure disorder, or a leukemia.

The invention further relates to a method of treating or reducing the severity of one or both of Fragile X syndrome (FXS) and Down syndrome (DS), or the symptoms thereof, in which the method comprises modulating the expression, expression level, amount, or activity of one or more genes as set forth in Table 5 in a cell and/or in a subject. The invention also relates to a method of treating or reducing the severity of one or both of Fragile X syndrome (FXS) and Down syndrome (DS), or the symptoms thereof, in a cell and/or in a subject, in which the method comprises administering or providing to the cell and/or to the subject an effective amount of an agent that modulates the expression, expression level, amount, or activity of one or more genes as set forth in Table 5 in the cell and/or in a subject. In an embodiment of the method, the agent is a polypeptide, a polynucleotide, a drug, or a small molecule compound. In an embodiment, the agent is a Fragile X mental retardation protein (FMRP) polypeptide, or a fragment thereof, or a polynucleotide sequence encoding FMRP, or a fragment thereof.

In an embodiment of the above-delineated methods and/or embodiments thereof, the expression, expression level, amount, or activity of the one or more genes of Table 5 is aberrant or abnormal in the cell and/or the subject relative to the expression, expression level, amount, or activity of the one or more genes in a normal, non-disease control. In an embodiment of the above-delineated methods and/or embodiments thereof, modulation of the expression, expression level, amount, or activity of the one or more genes of Table 5 comprises increasing or enhancing the expression, expression level, amount, or activity of the one or more genes in the cell and/or in the subject to treat or reduce the severity of the neurodevelopmental or neurodegenerative disease or disorder, or one or both of FXS, or DS. In an embodiment of the above-delineated methods and/or embodiments thereof, modulation of the expression, expression level, amount, or activity of the one or more genes of Table 5 comprises decreasing or reducing the expression, expression level, amount, or activity of said one or more genes in the cell and/or in the subject to treat or reduce the severity of the neurodevelopmental or neurodegenerative disease or disorder, or one or both of FXS, or DS. In an embodiment of the above-delineated methods and/or embodiments thereof, the cell is in vivo, in vitro, or ex vivo. In an embodiment of the above-delineated methods and/or embodiments thereof, the subject is a mammal. In an embodiment of the above-delineated methods and/or embodiments thereof, the subject is a human.

The invention is further based on the discovery that a gene as set forth in Table 5 (Appendix), or a polypeptide encoded by such gene, (e.g., the aberrant expression, expression level, amount, or activity of the gene) is associated with one or more neurodevelopmental or neurodevelopmental diseases or disorders, such as Fragile X syndrome (FXS) and/or Down syndrome (DS), and/or may be associated with a neurodegenerative disease or disorder, such as Alzheimer's disease, or a disease, disorder, or condition related to DS, e.g., autism, a seizure disorder, or a leukemia. In embodiments, the gene or the polypeptide encoded by a gene in Table 5 is aberrantly activated. In embodiments, the gene or the polypeptide encoded by the gene in Table 5 is aberrantly inactivated or its activity is functionally lost. In an embodiment, the provision or administration of Fragile X Mental Retardation Protein (FMRP) polypeptide or a polynucleotide encoding FMRP to a cell or to a subject in vitro, in vivo, or ex vivo modulates the aberrant activity of the gene of Table 5, or its encoded polypeptide in the cell or in the subject, thereby treating, alleviating, or ameliorating one or both of FXS, DS, and/or a symptom thereof. In an embodiment, an aberrant increase or enhancement in the expression level, expression, amount, or activity of a gene of Table 5 or the encoded polypeptide, is decreased or reduced in a cell or in a subject having, or having a propensity to have, one or more of Fragile X syndrome (FXS) or Down syndrome (DS) following the provision or administration of FMRP to a cell and/or to a subject. In another embodiment, an aberrant decrease or reduction in the expression level, expression, amount, or activity of the gene or the encoded polypeptide, is increased or enhanced in a cell or in a subject having, or having a propensity to have, one or more of Fragile X syndrome (FXS) or Down syndrome (DS), following the provision or administration of FMRP to a cell and/or to a subject. In an embodiment, the subject has, or has a propensity of having, FXS. In an embodiment, the subject has, or has a propensity of having, DS. In an embodiment, the subject has, or has a propensity of having, FXS and DS. In an embodiment, the subject has, or has a propensity of having, DS and a condition related thereto, such as autism, a seizure disorder, or a leukemia. In an embodiment, the subject is a mammal. In an embodiment, the subject is a human, e.g., a human patient. In embodiments, the cell is in vitro, in vivo, or ex vivo.

In accordance with the invention, genes (polynucleotides) associated with a developmental, neurodevelopmental or neurodegenerative disease or disorder were identified as also being associated with another developmental, neurodevelopmental or neurodegenerative disease or disorder. See, Example 7 and Table 5 (Appendix). Thus, a gene whose aberrant activity or expression was associated with one neurodevelopmental or neurodegenerative disease or disorder was further discovered to be associated with a different neurodevelopmental or neurodegenerative disease or disorder. (e.g., Columns A and B of Table 5). As shown in Table 5, a gene known in the context of one disease or disorder was also surprisingly found to be associated with another disease or disorder. In some cases, the neurodevelopmental or neurodegenerative disease or disorder associated with a given gene (polynucleotide) is both Fragile X syndrome (FXS) and Down syndrome (DS). In some cases, the neurodevelopmental or neurodegenerative disease or disorder associated with a given gene is Fragile X syndrome (FXS). In some cases, the neurodevelopmental or neurodegenerative disease or disorder associated with a given gene is Down syndrome (DS). Table 5 presents a list of genes (polynucleotides) and their association with one or more diseases, such as FXS and/or DS.

In embodiments, FMRP polypeptide, or a polynucleotide encoding FMRP, or a functional fragment thereof, is provided or administered to a cell and/or to a subject in which the expression, expression level, amount, or activity of a gene of Table 5, (or the encoded polypeptide product of the gene), is aberrantly increased or enhanced, or is aberrantly decreased or reduced. In such a case, the provision or administration of FMRP modulates the expression, expression level, amount, or activity of the gene of Table 5, (or the encoded polypeptide product of the gene), to reverse, decrease, or increase the aberrant activity of the gene, such that the neurological or neurodegenerative disease(s) or disorder(s) associated with the gene of Table 5, (or a polypeptide product of the gene), is treated, ameliorated, abrogated, reversed, or alleviated in the cell and/or in the subject. In embodiments, the subject is a mammal. In embodiments, the subject is a human subject. In embodiments, the cell is in vitro, in vivo, or ex vivo.

Table 5 of the Appendix presents genes, or a panel of genes, whose expression, expression level, amount, or activity is disrupted or aberrant in one or both of Fragile X syndrome (FXS) or Down syndrome (DS), wherein modulation of the expression, expression level, amount, or activity of said one or more genes treats or reduces the severity of one or both of FXS or DS. In an embodiment, modulation of the expression, expression level, amount, or activity of one or more genes in Table 5, or in the panel of genes, involves increasing or enhancing the expression, expression level, amount, or activity of the one or more genes to treat or reduce the severity of one or both of FXS or DS. In another embodiment, modulation of the expression, expression level, amount, or activity of one or more genes in Table 5, or in the panel of genes, involves decreasing or reducing the expression, expression level, amount, or activity of the one or more genes to treat or reduce the severity of one or both of FXS or DS. In an embodiment, Fragile X mental retardation protein (FMRP) polypeptide, or a fragment thereof, reverses or ameliorates the disrupted or aberrant expression, expression level, amount, or activity of one or more genes in Table 5 or in one or more genes of the gene panel.

As reported in detail below, Fragile X syndrome (FXS), driven by loss of the RNA binding protein FMRP, and Down syndrome (DS), driven by an extra copy of chromosome 21 (HSA21), are two common genetic causes of intellectual disability and autism that lack effective therapies. Defined for the first time herein are the global RNA targets of FMRP in human pluripotent stem cells and human cortical neurons, unexpectedly revealing that FMRP binds a substantial fraction of HSA21 encoded transcripts expressed in these cell types. Using patient cell lines, it was discovered that FXS and DS converge on a set of shared transcriptional and proteomic perturbations; some of the same genes are increased in abundance in both FXS and DS. Remarkably, acute upregulation of endogenous FMRP in DS patient cells through CRISPR activation (CRISPRa) is sufficient to significantly reduce protein expression levels of the key HSA21-encoded targets DYRK1A and APP. These results indicate that FXS and DS converge on a set of shared gene targets through alternative mechanisms, which may facilitate new opportunities for therapeutic intervention.

It was therefore hypothesized that FMRP target analyses in human pluripotent stem cells (hPSCs) and human excitatory cortical neurons would uncover novel facets of FMRP function with relevance to human biology. For the first time the global RNA targets of FMRP were analyzed in both cell types using enhanced crosslinking and immunoprecipitation followed by high-throughput sequencing (eCLIP-seq). In vitro derived cortical neurons have been extensively characterized at molecular and physiological levels and employed in multiple studies of neurodevelopmental disease associated genes. To enhance the specificity of the datasets, eCLIP-seq using isogenic FMR1^{y/-} hPSCs and FMR1^{y/-} neurons was also performed andnon-specific binding events were eliminated in each cell type through FMR1^{y/-} peak subtraction. These subtracted datasets provide high-confidence FMRP targets in two human cell types. It was unexpectedly discovered that FMRP targets were enriched for chromosome 21 (HSA21)-encoded transcripts, including targets strongly implicated in Down syndrome (DS) disease pathogenesis. DS is driven by a third copy of HSA21 which leads to a myriad of phenotypic effects including intellectual disability, elevated rates of autism, as well as elevated rates of Alzheimer's disease. Although FXS patients (1 in 4,000 males and 1 in 8,000 females) and DS patients (1 in 700) share some cognitive and behavioral deficits, a direct mechanistic connection between these diseases has not previously been reported.

Analyses of multiple FXS and DS patient cell lines revealed a set of shared transcriptional and proteomic perturbations. Moreover, protein expression levels of two key HSA21-encoded FMRP targets, DYRK1A and APP, in DS patient cells were significantly reduced by acutely upregulating endogenous FMRP through CRISPRa. Collectively, these results support a novel model whereby loss of FMRP in FXS and an extra copy of HSA21 in DS converge on a set of shared gene targets through different mechanisms (i.e., through loss of an RNA binding protein and an extra copy of a chromosome, respectively). Shared molecular mechanisms may facilitate new opportunities for therapeutic intervention.

### Fragile X Mental Retardation Protein (FMRP)

The RNA-binding protein Fragile X Mental Retardation Protein (FMRP) is encoded by the FMRP Translational Regulator 1 (FMR1) gene. Trinucleotide repeat expansion in the 5′ UTR of FMR1 leads to epigenetic silencing and loss of the encoded FMRP. FMRP is a multifunctional RNA-binding protein involved in mRNA splicing, stability, and transport. It can also repress mRNA translation by stalling ribosomal translocation during elongation. As described herein, FMRP in humans targets multiple transcripts encoded on chromosome 21. Further, as shown herein several FMRP targets are dysregulated in the absence of FMRP in both Fragile X syndrome and Down syndrome, thereby demonstrating that these syndromes converge on shared transcriptional and proteomic perturbations.

### Methods for FMRP-Based Therapy

The present invention provides methods of treating disease and/or disorders or symptoms thereof which comprise administering a therapeutically effective amount of a pharmaceutical composition comprising a compound (e.g., a polynucleotide encoding FMRP) of the formulae herein to a subject (e.g., a mammal such as a human). Thus, one embodiment is a method of treating a subject suffering from or susceptible to a disease or disorder or symptom thereof. The method includes the step of administering to the mammal a therapeutic amount of an amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme, or protein marker, karyotype, family history, and the like). The compounds herein may be also used in the treatment of any other disorders in which overexpression of a FMRP target is implicated.

The increased expression or activity of FMRP in a cell decreases expression of some FMRP targets (e.g., DYRK1A and APP). In some embodiments, the cell is a somatic cell. In some embodiments, cell is a brain cell, e.g., a glial cell or a neuron. The methods presented herein prevent or treat Down syndrome, Fragile X syndrome, and Alzheimer's disease. Down syndrome and Alzheimer's disease, for example, are characterized by increased expression or activity of DYRK1A and/or APP. Accordingly, the invention provides for the treatment of a variety of diseases and disorders associated with increased expression of targets of FMRP. In some embodiments, the invention provides for the treatment of a variety of diseases and disorders associated with increased expression of targets of FMRP that are encoded on chromosome 21. For example, increased APP, a FMRP target, has been observed in DS and Alzheimer's disease.

The invention generally features methods of increasing FMRP expression or activity in a subject having or at risk of developing Down syndrome, Fragile X syndrome, or Alzheimer's disease. Therapies provided by the invention include polypeptide therapies and polynucleotide therapies. In on embodiment, the method involves contacting a cell of the subject with a polynucleotide encoding FMRP or a fragment thereof, thereby increasing the expression or activity of FMRP in the cell. Polynucleotides encoding FMRP or a fragment thereof can be encoded in a plasmid or expression vector. In some embodiments, the polynucleotide encoding FMRP or a fragment thereof is an RNA polynucleotide.

Polynucleotides encoding FMRP or a fragment thereof can be used to express the FMRP protein or a fragment thereof in a subject. Alternatively, cells can be contacted with polynucleotides encoding FMRP or a fragment thereof, and then the cells are administered to the subject.

In one aspect, cells can be transfected with a polynucleotide to express a FMRP protein or a fragment thereof using an ex vivo approach in which cells are removed from a patient, transfected by e.g., electroporation or lipofection, and re-introduced to the patient. In some embodiments, cells are transduced, rather than transfected, with a viral vector encoding FMRP or a fragment thereof. In some embodiments, the viral vector is a lentiviral vector, an adenoviral vector, or an adeno-associated viral vector. Other viral vectors are contemplated, and the vector used may be dependent on the cell type to be transduced.

In various embodiments, the level, expression, or activity of FMRP in a cell in vivo is transiently increased. In various embodiments, this is accomplished by administering an agent (e.g., a polynucleotide encoding FMRP or a fragment thereof). In various embodiments, the level, expression, or activity of FMRP in a cell is increased for about 3 months, 2 months, 1 month or less after administration of the agent. In various embodiments, the level, expression, or activity of FMRP in a cell is increased for about 4 weeks, 3 weeks, 2 weeks, 1 week or less after administration of the agent. In various embodiments, the level, expression, or activity of FMRP in a cell is increased for about 7 days, 6 days, 5 days, 4 days, 3 days, 2 days, 1 day or less after administration of the agent. In various embodiments, the level, expression, or activity of FMRP in a cell is increased for about 24, 23, 22, 21, 20, 19, 18, 17, 16, 15, 14, 13, 12, 11, 10, 9, 8, 7, 6, 5, 4, 3, 2, or 1 hour or less after administration of the agent. In various embodiments, the level, expression, or activity of FMRP in a cell is increased for about 60, 45, 30, 15, 10, 9, 8, 7, 6, 5, 4, 3, 2, or 1 minute or less after administration of the agent. In various embodiments, the increase in the level, expression, or activity of FMRP in a cell is not constitutive.

The invention also provides methods of binding an FMRP target in a cell. The method involves contacting the cell with a FMRP polypeptide or a polynucleotide encoding FMRP. In some embodiments, binding to the FMRP target is increased when the FMRP polypeptide is heterodimerized with a Fragile X mental retardation syndrome related 1 protein (FXR1P). In some embodiments, the polynucleotide encoding FMRP also encodes FXR1P. In some embodiments, the polynucleotide of the present invention encodes a fragment of FMRP and/or a fragment of FXR1P. In some embodiments, FXR1P is encoded on a second polynucleotide. Thus, in one embodiment of a method of binding an FMRP target in a cell, the method comprises contacting the cell with a first polynucleotide encoding FMRP and a second nucleotide encoding FXR1P.

### Delivery of Polynucleotides

Naked polynucleotides, or analogs thereof, are capable of entering mammalian cells and inhibiting expression of a gene of interest. Nonetheless, it may be desirable to utilize a formulation that aids in the delivery of polynucleotides, oligonucleotides, or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference). Polynucleotides may be delivered via transfection or transduction as described supra.

Polynucleotides encoding FMRP or a fragment thereof can be administered to a target tissue in vivo as a single dose or in multiple doses (e.g., sequentially). The expression desired for FMRP in vivo can be tailored by altering the frequency of administration and/or the amount of the polynucleotide encoding the protein, or a fragment thereof, administered to a subject or used to contact a cell. Because the polynucleotide encoding FMRP administered to a subject is degraded over time, one of skill in the art can remove or stop the in vivo protein expression by halting further administrations and permitting degradation of the polynucleotide encoding FMRP.

### Transfection Reagents

In certain embodiments of the aspects described herein, a polynucleotide encoding FMRP can be introduced into a target tissue in vivo by transfection or lipofection. Suitable agents for transfection or lipofection include, for example but are not limited to, calcium phosphate, DEAE dextran, lipofectin, lipofectamine, DIMRIE C™, SUPERFECT™, and EFFECTIN™ (Qiagen™), UNIFECTIN™, MAXIFECTIN™, DOTMA, DOGS™ (Transfectam; dioctadecylamidoglycylspermine), DOPE (1,2-dioleoyl-sn-glycero-3-phosphoethanolamine), DOTAP (1,2-dioleoyl-3-trimethylammonium propane), DDAB (dimethyl dioctadecylammonium bromide), DHDEAB (N,N-di-n-hexadecyl-N,N-dihydroxyethyl ammonium bromide), HDEAB (N-n-hexadecyl-N,N-dihydroxyethylammonium bromide), polybrene, poly(ethylenimine) (PEI), and the like. (See, e.g., Banerjee et al., Med. Chem. 42:4292-99 (1999); Godbey et al., Gene Ther. 6:1380-88 (1999); Kichler et al., Gene Ther. 5:855-60 (1998); Birchaa et al., J. Pharm. 183:195-207 (1999)).

A FMRP polypeptide or polynucleotide can be transfected into a target tissue in vivo as disclosed herein as a complex with cationic lipid carriers (e.g., OLIGOFECTAMINE™) or non-cationic lipid-based carriers (e.g., Transit-TKOTM™, Mirus Bio LLC, Madison, Wis.). Successful introduction of a FMRP polypeptide or polynucleotide into a target tissue in vivo can be monitored using various known methods. Successful transfection of a target tissue in vivo with a FMRP polypeptide or polynucleotide can be determined by measuring the protein expression level of the target polypeptide by e.g., Western blotting or immunocytochemistry.

In some embodiments of the aspects described herein, the FMRP polypeptide or polynucleotide is introduced into a target tissue in vivo using a transfection reagent. Some exemplary transfection reagents include, for example, cationic lipids, such as lipofectin (Junichi et al, U.S. Pat. No. 5,705,188), cationic glycerol derivatives, and polycationic molecules, such as polylysine (Lollo et al., PCT Application WO 97/30731). Examples of commercially available transfection reagents include, for example Lipofectamine™ (Invitrogen; Carlsbad, Calif), Lipofectamine 2000™ (Invitrogen; Carlsbad, Calif), 293FECTIN™ (Invitrogen; Carlsbad, Calif), CELLFECTIN™ (Invitrogen; Carlsbad, Calif), DMRIE-C™ (Invitrogen; Carlsbad, Calif), FREESTYLE™ MAX (Invitrogen; Carlsbad, Calif), Lipofectamine™ 2000 CD (Invitrogen; Carlsbad, Calif), Lipofectamine™ (Invitrogen; Carlsbad, Calif.), RNAiMAX (Invitrogen; Carlsbad, Calif), OLIGOFECTAMINE™ (Invitrogen; Carlsbad, Calif.), OPTIFECT™ (Invitrogen; Carlsbad, Calif), X-tremeGENE Q2 Transfection Reagent (Roche; Grenzacherstrasse, Switzerland), DOTAP Liposomal Transfection Reagent (Grenzacherstrasse, Switzerland), DOSPER Liposomal Transfection Reagent (Grenzacherstrasse, Switzerland), or Fugene (Grenzacherstrasse, Switzerland), Transfectam® Reagent (Promega; Madison, Wis.), TRANSFAST™ Transfection Reagent (Promega; Madison, Wis.), TFX™-20 Reagent (Promega; Madison, Wis.), TFX™-50 Reagent (Promega; Madison, Wis.), DREAMFECT™ (OZ Biosciences; Marseille, France), EcoTransfect (OZ Biosciences; Marseille, France), TransPass.sup.a D1 Transfection Reagent (New England Biolabs; Ipswich, Mass., USA), LYOVEC™/LIPOGEN™ (Invitrogen; San Diego, Calif, USA), PerFectin Transfection Reagent (Genlantis; San Diego, Calif, USA), NeuroPORTER Transfection Reagent (Genlantis; San Diego, Calif, USA), GenePORTER Transfection reagent (Genlantis; San Diego, Calif, USA), GenePORTER 2 Transfection reagent (Genlantis; San Diego, Calif, USA), Cytofectin Transfection Reagent (Genlantis; San Diego, Calif, USA), BaculoPORTER Transfection Reagent (Genlantis; San Diego, Calif, USA), TroganPORTERT™ transfection Reagent (Genlantis; San Diego, Calif, USA), RiboFect (Bioline; Taunton, Mass., USA), PlasFect (Bioline; Taunton, Mass., USA), UniFECTOR (B-Bridge International; Mountain View, Calif, USA), SureFECTOR (B-Bridge International; Mountain View, Calif USA), or HIFECT™ (B-Bridge International, Mountain View, Calif, USA), among others.

In other embodiments, highly branched organic compounds, termed “dendrimers,” can be used to bind the exogenous nucleic acid, such as the FMRP polynucleotide described herein, and introduce it into a target tissue in vivo.

In other embodiments, cell penetrating peptides can be used to bind and transport the FMRP polynucleotide described herein, into a target tissue in vivo.

In other embodiments of the aspects described herein, non-chemical methods of transfection are contemplated. Such methods include, but are not limited to, electroporation (methods whereby an instrument is used to create micro-sized holes transiently in the plasma membrane of cells under an electric discharge), sono-poration (transfection via the application of sonic forces to cells), and optical transfection (methods whereby a tiny (about 1 μm diameter) hole is transiently generated in the plasma membrane of a cell using a highly focused laser). In other embodiments, particle-based methods of transfections are contemplated, such as the use of a gene gun, whereby the nucleic acid is coupled to a nanoparticle of an inert solid (commonly gold) which is then “shot” directly into the target cell's nucleus; “magnetofection,” which refers to a transfection method, that uses magnetic force to deliver exogenous nucleic acids coupled to magnetic nanoparticles into target cells; “impalefection,” which is carried out by impaling cells by elongated nanostructures, such as carbon nanofibers or silicon nanowires which have been coupled to exogenous nucleic acids.

Other agents may be utilized to enhance the penetration of the administered nucleic acids, including glycols, such as ethylene glycol and propylene glycol, pyrrols such as 2-pyrrol, azones, and terpenes, such as limonene and menthone.

### Delivery Formulations and Pharmaceutical Compositions

In some embodiments, a FMRP polypeptide or fragment thereof or a polynucleotide encoding a FMRP polypeptide or fragment thereof is delivered to a target tissue in vivo encapsulated in a nanoparticle. Methods for nanoparticle packaging are well known in the art, and are described, for example, in Bose S, et al (Role of Nucleolin in Human Parainfluenza Virus Type 3 Infection of Human Lung Epithelial Cells. J. Virol. 78:8146. 2004); Dong Y et al. Poly(d,l-lactide-co-glycolide)/montmorillonite nanoparticles for oral delivery of anticancer drugs. Biomaterials 26:6068. 2005); Lobenberg R. et al (Improved body distribution of 14C-labelled AZT bound to nanoparticles in rats determined by radioluminography. J Drug Target 5:171. 1998); Sakuma S R et al (Mucoadhesion of polystyrene nanoparticles having surface hydrophilic polymeric chains in the gastrointestinal tract. Int J Pharm 177:161. 1999); Virovic L et al. Novel delivery methods for treatment of viral hepatitis: an update. Expert Opin Drug Deliv 2:707. 2005); and Zimmermann E et al, Electrolyte- and pH-stabilities of aqueous solid lipid nanoparticle (SLN) dispersions in artificial gastrointestinal media. Eur J Pharm Biopharm 52:203. 2001). In some embodiments, where the composition comprises more than one FMRP polypeptide or FMRP polynucleotide, each FMRP polypeptide or polynucleotide is formulated as its own nanoparticle formulation and the pharmaceutical composition comprises a plurality of FMRP polypeptide or FMRP polynucleotide-nanoparticle formulations. Each method represents a separate embodiment of the present invention.

In some embodiment, one or more FMRP polypeptides or polynucleotides encoding a FMRP polypeptide is delivered to a target tissue in vivo in a vesicle, e.g., a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid). In some embodiments, where the composition comprises more than one FMRP polypeptide or polynucleotide, each FMRP polypeptide or polynucleotide can be formulated as its own liposome formulation, and a pharmaceutical composition can comprise a plurality of FMRP polypeptide or polynucleotide-liposome formulations.

In some embodiments, compositions comprising at least one FMRP polypeptide or polynucleotide for delivery to a target tissue in vivo as disclosed herein can be, in another embodiment, administered to a subject by any method known to a person skilled in the art, such as parenterally, intramuscularly, intra-dermally, subcutaneously, intraperitonealy, or intra-ventricularly. In another embodiment of methods and compositions of the present invention, compositions comprising at least one FMRP polypeptide or polynucleotide for delivery to a target tissue in vivo as disclosed herein are formulated in a form suitable for injection, i.e., as a liquid preparation. Suitable liquid formulations include solutions, suspensions, dispersions, emulsions, oils and the like. In another embodiment of the present invention, the active ingredient is formulated in a capsule, e.g., a slow release capsule.

In other embodiments, the pharmaceutical compositions comprising at least one FMRP polypeptide or polynucleotide for delivery to a target tissue in vivo as disclosed herein can be administered by intra-arterial, or intramuscular injection of a liquid preparation. Suitable liquid formulations include solutions, suspensions, dispersions, emulsions, oils and the like. In another embodiment, the pharmaceutical compositions comprising at least one FMRP polypeptide or polynucleotide as disclosed herein can be administered intra-arterially and are thus formulated in a form suitable for intra-arterial administration.

In another embodiment, a pharmaceutical composition comprising at least one FMRP polypeptide or polynucleotide for delivery to a target tissue in vivo as disclosed herein can be administered topically to body surfaces and are thus formulated in a form suitable for topical administration. Suitable topical formulations include gels, ointments, creams, lotions, drops and the like. For topical administration, the compositions or their physiologically tolerated derivatives are prepared and applied as solutions, suspensions, or emulsions in a physiologically acceptable diluent with or without a pharmaceutical carrier.

As used herein “pharmaceutically acceptable carriers or diluents” are well known to those skilled in the art. The carrier or diluent may be may be, in various embodiments, a solid carrier or diluent for solid formulations, a liquid carrier or diluent for liquid formulations, or mixtures thereof. In another embodiment, solid carriers/diluents include, but are not limited to, a gum, a starch (e.g. corn starch, pregeletanized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof. In other embodiments, pharmaceutically acceptable carriers for liquid formulations may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of petroleum, animal, vegetable, or synthetic origin, for example, peanut oil, soybean oil, mineral oil, olive oil, sunflower oil, and fish-liver oil.

In another embodiment, a compositions for delivery of a FMRP polypeptide or polynucleotide to a target tissue in vivo further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmelose sodium, crospovidone, guar gum, sodium starch glycolate), buffers (e.g., Tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g., hydroxypropyl cellulose, hyroxypropylmethyl cellulose), viscosity increasing agents (e.g., carbomer, colloidal silicon dioxide, ethyl cellulose, guar gum), sweeteners (e.g., aspartame, citric acid), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates) and/or adjuvants. Each of the above excipients represents a separate embodiment of the present invention.

In another embodiment, a pharmaceutical composition for delivery of a FMRP polypeptide or polynucleotide to a target tissue in vivo can comprise a FMRP polypeptide or polynucleotide in a controlled-release composition, i.e., a composition in which the compound is released over a period of time after administration. Controlled- or sustained-release compositions include formulation in lipophilic depots (e.g., fatty acids, waxes, oils). In another embodiment, a composition for delivery of a FMRP polypeptide or polynucleotide to a target tissue in vivo is an immediate-release composition, i.e., a composition in which the entire compound is released immediately after administration.

In another embodiment, for delivery of a FMRP polypeptide or polynucleotide to a target tissue in vivo, one can modify a FMRP polypeptide or polynucleotide of the present invention by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds (Abuchowski et al., 1981; Newmark et al., 1982; and Katre et al., 1987). Such modifications also increase, in another embodiment, the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

In another embodiment, a composition for delivery of a FMRP polypeptide or polynucleotide to a target tissue in vivo is formulated to include a neutralized pharmaceutically acceptable salt form. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like. Each of the above additives, excipients, formulations and methods of administration represents a separate embodiment of the present invention.

In some embodiments of the aspects described herein, involving in vivo administration of FMRP polypeptide or polynucleotide or compositions thereof to a target tissue in vivo, are formulated in conjunction with one or more penetration enhancers, surfactants and/or chelators. Suitable surfactants include fatty acids and/or esters or salts thereof, bile acids and/or salts thereof. Suitable bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether.

A composition comprising at least one FMRP polypeptide or polynucleotide as disclosed herein can be formulated into any of many possible administration forms, including a sustained release form. The compositions can be formulated as suspensions in aqueous, non-aqueous or mixed media. Aqueous suspensions can further contain substances which increase the viscosity of the suspension including, for example, sodium carboxymethylcellulose, sorbitol and/or dextran. The suspension can also contain stabilizers.

A composition comprising at least one FMRP polypeptide or polynucleotide as disclosed herein can be prepared and formulated as emulsions for the delivery of polypeptides or polynucleotides. Emulsions are typically heterogeneous systems of one liquid dispersed in another in the form of droplets usually exceeding 0.1 μm in diameter (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions can be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions can contain further components in addition to the dispersed phases, and the active agent (i.e., FMRP polypeptide or polynucleotide), which can be present as a solution in either the aqueous phase, oily phase or itself as a separate phase. Pharmaceutical excipients such as emulsifiers, stabilizers, dyes, and anti-oxidants can also be present in emulsions as needed. Emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise, a system of oil droplets enclosed in globules of water stabilized in an oily continuous phase provides an o/w/o emulsion. Emulsifiers can broadly be classified into four categories: synthetic surfactants, naturally occurring emulsifiers, absorption bases, and finely dispersed solids (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

As noted above, liposomes can optionally be prepared to contain surface groups to facilitate delivery of liposomes and their contents to specific cell populations. For example, a liposome can comprise a surface groups such as antibodies or antibody fragments, small effector molecules for interacting with cell-surface receptors, antigens, and other like compounds.

Surface groups can be incorporated into the liposome by including in the liposomal lipids a lipid derivatized with the targeting molecule, or a lipid having a polar-head chemical group that can be derivatized with the targeting molecule in preformed liposomes. Alternatively, a targeting moiety can be inserted into preformed liposomes by incubating the preformed liposomes with a ligand-polymer-lipid conjugate.

A number of liposomes comprising nucleic acids are known in the art. WO 96/40062 (Thierry et al.) discloses methods for encapsulating high molecular weight nucleic acids in liposomes. U.S. Pat. No. 5,264,221 (Tagawa et al.) discloses protein-bonded liposomes and asserts that the contents of such liposomes can include an RNA molecule. U.S. Pat. No. 5,665,710 (Rahman et al.) describes certain methods of encapsulating oligodeoxynucleotides in liposomes. WO 97/04787 (Love et al.) discloses liposomes comprising RNAi molecules targeted to the raf gene. In addition, methods for preparing a liposome composition comprising a nucleic acid can be found in e.g., U.S. Pat. Nos. 6,011,020; 6,074,667; 6,110,490; 6,147,204; 6, 271, 206; 6,312,956; 6,465,188; 6,506,564; 6,750,016; and 7,112,337. Each of these approaches can provide delivery of a FMRP polynucleotide as described herein to a cell.

In some embodiments of the aspects described herein, a composition comprising at least one FMRP polypeptide or polynucleotide for in vivo protein expression in a target tissue as disclosed herein can be encapsulated in a nanoparticle. Methods for nanoparticle packaging are well known in the art, and are described, for example, in Bose S, et al (Role of Nucleolin in Human Parainfluenza Virus Type 3 Infection of Human Lung Epithelial Cells. J. Virol. 78:8146. 2004); Dong Y et al. Poly(d,l-lactide-co-glycolide)/montmorillonite nanoparticles for oral delivery of anticancer drugs. Biomaterials 26:6068. 2005); Lobenberg R. et al (Improved body distribution of 14C-labelled AZT bound to nanoparticles in rats determined by radioluminography. J Drug Target 5:171.1998); Sakuma S R et al (Mucoadhesion of polystyrene nanoparticles having surface hydrophilic polymeric chains in the gastrointestinal tract. Int J Pharm 177:161. 1999); Virovic L et al. Novel delivery methods for treatment of viral hepatitis: an update. Expert Opin Drug Deliv 2:707.2005); and Zimmermann E et al, Electrolyte- and pH-stabilities of aqueous solid lipid nanoparticle (SLN) dispersions in artificial gastrointestinal media. Eur J Pharm Biopharm 52:203. 2001), the contents of which are herein incorporated in their entireties by reference.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: High-Confidence FMRP RNA Targets in hPSCs and Neurons Defined by eCLIP-seq

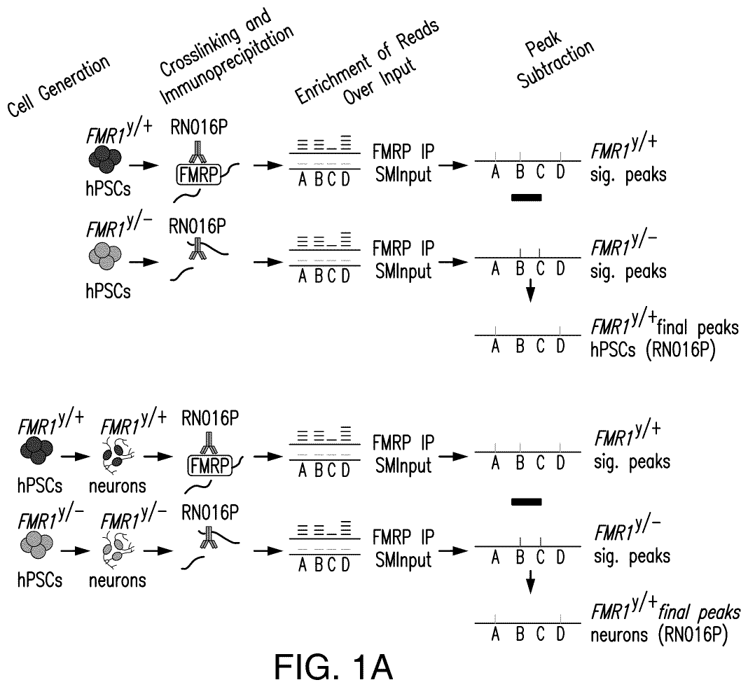

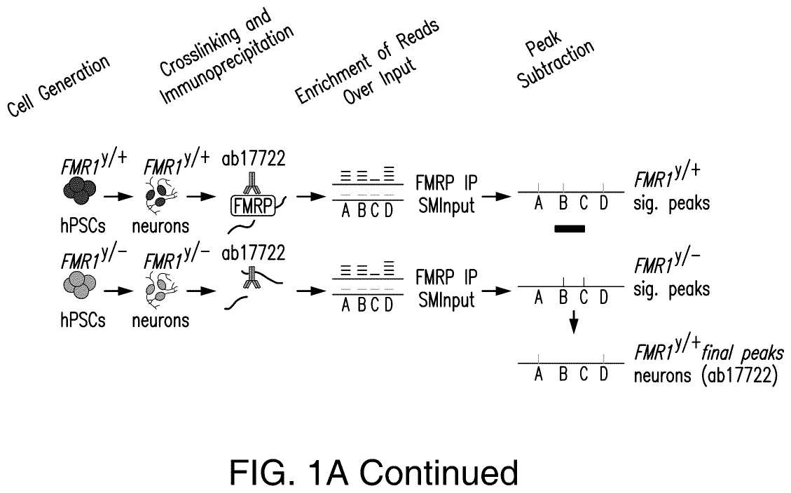

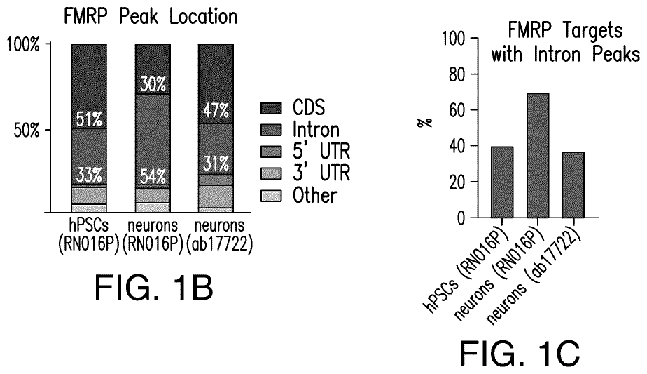

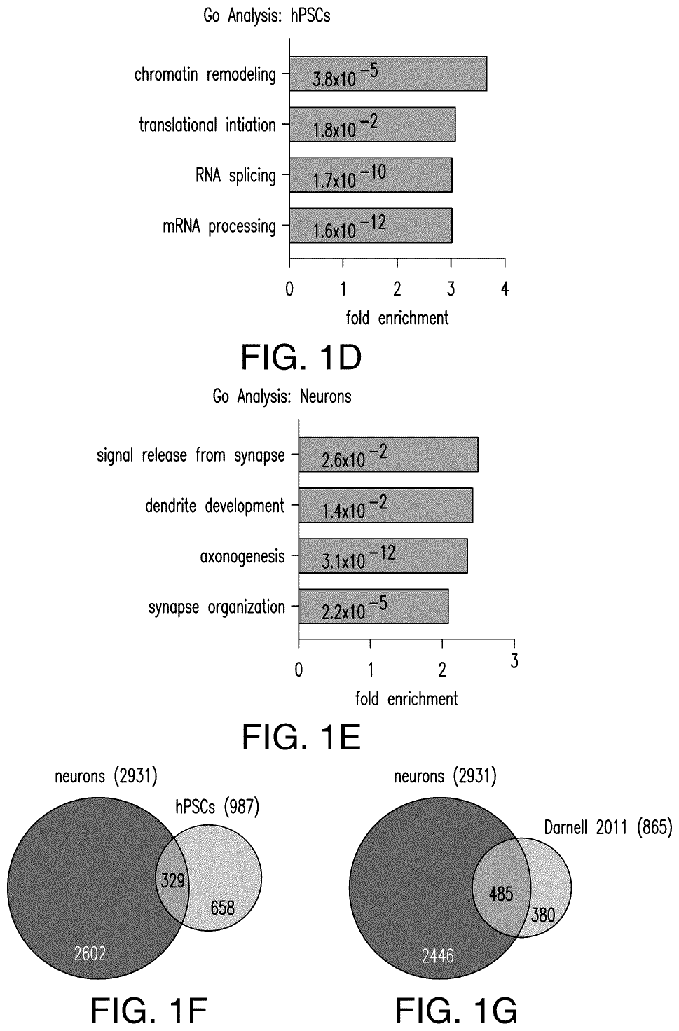

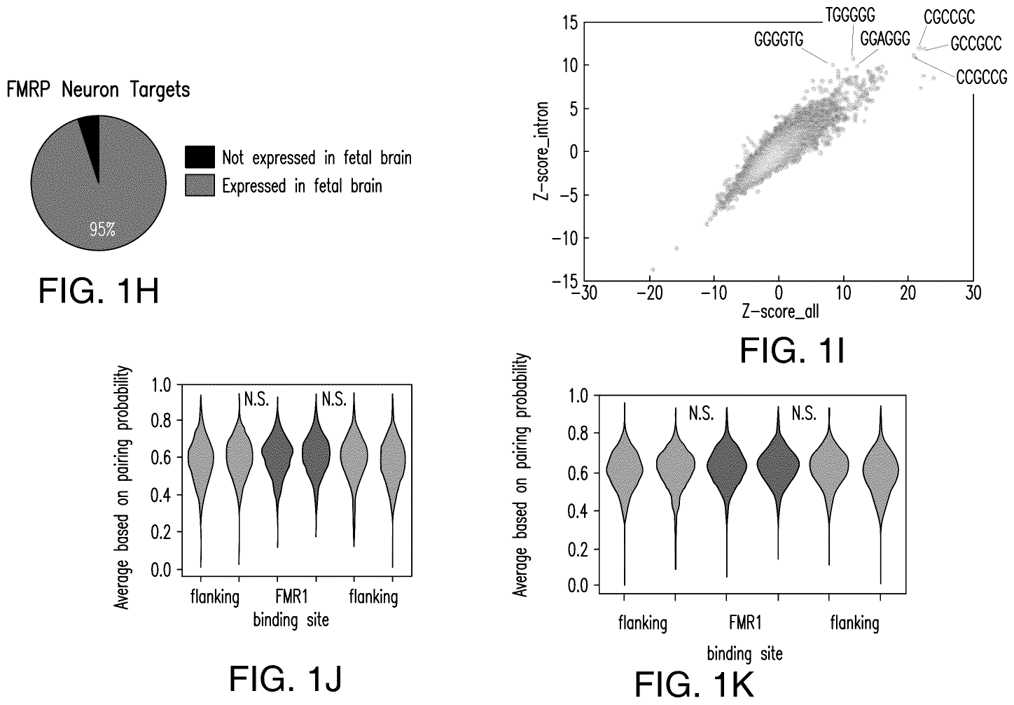

To define the RNA targets of FMRP in both hPSCs and excitatory cortical neurons, eCLIP-seq was used with two independent anti-FMRP antibodies (RN016P (MBL International) and ab17722 (Abcam)) (FIG. 1A, Table 1), both of which have previously been used for FMRP eCLIP-seq in non-neuronal cell types. For all eCLIP-seq experiments, significant peaks were identified by comparing peak read density between eCLIP and a paired size-matched input (SMInput) (FIG. 1A), excluding PCR duplicates and considering only uniquely mapped reads as previously described (Van Nostrand, E. L. et al., Nature methods 13, 508-14 (2016). A stringent peak threshold of at least 8-fold enrichment in immunoprecipitation over the SMlnput with p-values ≤10⁻³ was used. To reduce potential non-specific binding events, the same eCLIP-seq experiments were performed utilizing isogenic FMR1^{y/-} and neurons and significant peaks detected in each dataset were subtracted from the corresponding FMR1^{y/+} datasets (FIG. 1A, Table 1). Importantly, FMR1^{y/-} peak subtraction eliminated 18-33% of FMRP RNA targets from each FMR1^{y/+} dataset (Table 1), focusing the analyses on targets with highest specificity.

TABLE 1

| | | Significant | | | % |
|---|---|---|---|---|---|
| Cell Type | Antibody | Peaks | Genes | Cell Input | Subtracted |
| hPSCs | RN016P | 1688 | 987 | 1.5E+07 | 30.2 |
| neurons | RN016P | 363 | 93 | 1.5E+07 | 33.1 |
| neurons | ab17722 | 6634 | 2889 | 7.0E+07 | 17.8 |

All subsequent analyses were performed using significant peaks and gene targets that remained following FMR1^{y/-} peak subtraction (Table 1). Of note, the FMRP antibody RN016P (MBL Intl.) neuron datasets were generated using a smaller number of neurons as input compared with the FMRP antibody ab17722 (Abcam) neuron datasets, and thus yielded fewer significant peaks and genes.

Across all FMRP eCLIP-seq datasets, a consistent pattern of peak distributions was observed, with the majority of peaks located in CDS and introns and the remaining peaks distributed across other gene elements (FIG. 1B). Intronic peaks were found in roughly 40% of all FMRP targets (FIG. 1C), consistent with FMRP binding these targets as unspliced pre-mRNAs. Several studies previously identified FMRP RNA targets in mouse brain but did not report the presence of intron binding events, which may be due in part to polysome enrichment employed in two of these studies. Consistent with the datasets described herein, four separate studies of FMRP RNA targets in human cells or tissue including HEK293T cells, K562 cells, and human postmortem brain all detected FMRP intron binding events.

Gene Ontology (GO) analysis revealed FMRP targets that are themselves involved in different aspects of RNA and DNA regulation in hPSCs, including chromatin remodeling and RNA splicing (FIG. 1D, Table 2). In neurons, not surprisingly, GO analysis pointed to targets related to neuronal development (FIG. 1E, Table 2). FMRP targets in hPSCs and neurons were minimally overlapping (FIG. 1F), indicative of largely distinct functions of FMRP in pluripotent stem cells versus post-mitotic neurons. Interestingly, while 56.07% of FMRP targets previously identified in mouse brain (Darnell, J. C. et al., Cell 146, 247-261 (2011)) were FMRP targets inhuman neurons, representing a 2.93-fold enrichment over expected (p=3.92×10⁻¹³⁸), 83.5% of human neuron targets did not overlap with those previously identified in mouse brain¹ (FIG. 1F), suggesting that existing knowledge of FMRP target biology remains incomplete. Notably, the vast majority (95%) of FMRP targets identified in human neuron in vitro were also detectably expressed in human fetal brain tissue (FIG. 1G), supporting the in vivo relevance of the neuronal transcriptomes. Collectively, these data define high-confidence FMRP RNA targets for the first time in hPSCs and neurons and identify FMRP binding events on pre-mRNA targets in both cell types.

TABLE 2
GO Analysis

| Analysis Type: | PANTHER Overrepresentation Test (Released 20190711) | | | | | |
|---|---|---|---|---|---|---|
| Annotation Version and Release Date: | GO Ontology database Released 2019 Oct. 8 | | | | | |
| Analyzed List: | upload_1 (Homo sapiens) | | | | | |
| Reference List: | stem cell universe.csv (Homo sapiens) | | | | | |
| Test Type: | FISHER | | | | | |
| Correction: | BONFERRONI | | | | | |
| Bonferroni count: | 7847 | | | | | |
| | stem cell | | up- | up- | upload_ | |
| | universe. | up- | load_ | load_ | 1 | upload_ |
| | csv- | load_ | 1 | 1 | (fold | 1 |
| | REFLIST | 1 | (ex- | (over/ | Enrich- | (P- |
| GO biological process complete | (14063) | (858) | pected) | under) | ment) | value) |
| posttranscriptional gene silencing by RNA | 57 | 16 | 3.48 | + | 4.6 | 2.33E−02 |
| (GO: 0035194) | | | | | | |
| posttranscriptional gene silencing | 58 | 16 | 3.54 | + | 4.52 | 2.82E−02 |
| (GO: 0016441) | | | | | | |
| chromatin remodeling (GO: 0006338) | 143 | 32 | 8.72 | + | 3.67 | 3.76E−05 |
| regulation of mRNA processing (GO: 0050684) | 131 | 28 | 7.99 | + | 3.5 | 7.90E−04 |
| regulation of RNA splicing (GO: 0043484) | 128 | 25 | 7.81 | + | 3.2 | 1.60E−02 |
| mRNA transport (GO: 0051028) | 144 | 28 | 8.79 | + | 3.19 | 4.27E−03 |
| gene silencing (GO: 0016458) | 131 | 25 | 7.99 | + | 3.13 | 2.29E−02 |
| translational initiation (GO: 0006413) | 138 | 26 | 8.42 | + | 3.09 | 1.80E−02 |
| RNA transport (GO: 0050658) | 178 | 33 | 10.86 | + | 3.04 | 1.12E−03 |
| nucleic acid transport (GO: 0050657) | 178 | 33 | 10.86 | + | 3.04 | 1.12E−03 |
| RNA splicing via transesterification reactions | 281 | 52 | 17.14 | + | 3.03 | 3.00E−07 |
| (GO: 0000375) | | | | | | |
| RNA splicing (GO: 0008380) | 373 | 69 | 22.76 | + | 3.03 | 1.69E−10 |
| mRNA processing (GO: 0006397) | 435 | 80 | 26.54 | + | 3.01 | 1.64E−12 |
| mRNA splicing via spliceosome (GO: 0000398) | 278 | 51 | 16.96 | + | 3.01 | 6.11E−07 |
| RNA splicing via transesterification reactions | 278 | 51 | 16.96 | + | 3.01 | 6.11E−07 |
| with bulged adenosine as nucleophile | | | | | | |
| (GO: 0000377) | | | | | | |
| establishment of RNA localization | 180 | 33 | 10.98 | + | 3 | 1.41E−03 |
| (GO: 0051236) | | | | | | |
| regulation of gene expression epigenetic | 210 | 38 | 12.81 | + | 2.97 | 2.25E−04 |
| (GO: 0040029) | | | | | | |
| mitotic prometaphase (GO: 0000236) | 167 | 30 | 10.19 | + | 2.94 | 7.35E−03 |
| RNA localization (GO: 0006403) | 200 | 35 | 12.2 | + | 2.87 | 1.61E−03 |
| cytoskeleton-dependent intracellular | 166 | 28 | 10.13 | + | 2.76 | 4.88E−02 |
| transport (GO: 0030705) | | | | | | |
| regulation of mRNA metabolic process | 298 | 49 | 18.18 | + | 2.7 | 4.39E−05 |
| (GO: 1903311) | | | | | | |
| mRNA metabolic process (GO: 0016071) | 640 | 105 | 39.05 | + | 2.69 | 3.89E−14 |
| rhythmic process (GO: 0048511) | 217 | 35 | 13.24 | + | 2.64 | 1.10E−02 |
| nucleobase-containing compound transport | 217 | 35 | 13.24 | + | 2.64 | 1.10E−02 |
| (GO: 0015931) | | | | | | |
| positive regulation of cell cycle process | 244 | 37 | 14.89 | + | 2.49 | 2.34E−02 |
| (GO: 0090068) | | | | | | |
| regulation of chromosome organization | 321 | 46 | 19.58 | + | 2.35 | 4.59E−03 |
| (GO: 0033044) | | | | | | |
| RNA processing (GO: 0006396) | 806 | 113 | 49.17 | + | 2.3 | 5.16E−11 |
| chromatin organization (GO: 0006325) | 616 | 85 | 37.58 | + | 2.26 | 3.03E−07 |
| covalent chromatin modification | 341 | 46 | 20.8 | + | 2.21 | 2.84E−02 |
| (GO: 0016569) | | | | | | |
| regulation of mitotic cell cycle phase | 390 | 51 | 23.79 | + | 2.14 | 1.52E−02 |
| transition (GO: 1901990) | | | | | | |
| mitotic cell cycle process (GO: 1903047) | 573 | 74 | 34.96 | + | 2.12 | 9.05E−05 |
| posttranscriptional regulation of gene | 487 | 62 | 29.71 | + | 2.09 | 2.37E−03 |
| expression (GO: 0010608) | | | | | | |
| chromosome organization (GO: 0051276) | 940 | 117 | 57.35 | + | 2.04 | 2.58E−08 |
| regulation of cell cycle process (GO: 0010564) | 670 | 82 | 40.88 | + | 2.01 | 1.42E−04 |
| mitotic cell cycle (GO: 0000278) | 647 | 79 | 39.47 | + | 2 | 3.44E−04 |
| RNA metabolic process (GO: 0016070) | 1483 | 177 | 90.48 | + | 1.96 | 5.68E−13 |
| negative regulation of cellular macromolecule | 1118 | 132 | 68.21 | + | 1.94 | 2.87E−08 |
| biosynthetic process (GO: 2000113) | | | | | | |
| symbiotic process (GO: 0044403) | 682 | 80 | 41.61 | + | 1.92 | 1.14E−03 |
| negative regulation of macromolecule | 1181 | 138 | 72.05 | + | 1.92 | 1.61E−08 |
| biosynthetic process (GO: 0010558) | | | | | | |
| viral process (GO: 0016032) | 626 | 73 | 38.19 | + | 1.91 | 5.22E−03 |
| negative regulation of cellular biosynthetic | 1228 | 142 | 74.92 | + | 1.9 | 1.59E−08 |
| process (GO: 0031327) | | | | | | |
| negative regulation of nucleobase-containing | 1152 | 133 | 70.28 | + | 1.89 | 8.84E−08 |
| compound metabolic process (GO: 0045934) | | | | | | |
| nucleic acid metabolic process (GO: 0090304) | 2026 | 233 | 123.61 | + | 1.88 | 6.52E−17 |
| regulation of mitotic cell cycle (GO: 0007346) | 575 | 66 | 35.08 | + | 1.88 | 2.73E−02 |
| microtubule-based process (GO: 0007017) | 576 | 66 | 35.14 | + | 1.88 | 2.82E−02 |
| negative regulation of RNA metabolic process | 1061 | 121 | 64.73 | + | 1.87 | 1.99E−06 |
| (GO: 0051253) | | | | | | |
| negative regulation of biosynthetic process | 1251 | 142 | 76.32 | + | 1.86 | 5.73E−08 |
| (GO: 0009890) | | | | | | |
| interspecies interaction between organisms | 705 | 80 | 43.01 | + | 1.86 | 4.08E−03 |
| (GO: 0044419) | | | | | | |
| negative regulation of gene expression | 1403 | 156 | 85.6 | + | 1.82 | 1.57E−08 |
| (GO: 0010629) | | | | | | |
| negative regulation of nucleic acid-templated | 981 | 109 | 59.85 | + | 1.82 | 7.53E−05 |
| transcription (GO: 1903507) | | | | | | |
| negative regulation of RNA biosynthetic | 982 | 109 | 59.91 | + | 1.82 | 7.65E−05 |
| process (GO: 1902679) | | | | | | |
| gene expression (GO: 0010467) | 1784 | 198 | 108.84 | + | 1.82 | 5.45E−12 |
| negative regulation of transcription DNA- | 938 | 103 | 57.23 | + | 1.8 | 2.95E−04 |
| templated (GO: 0045892) | | | | | | |
| cellular response to DNA damage stimulus | 730 | 80 | 44.54 | + | 1.8 | 1.38E−02 |
| (GO: 0006974) | | | | | | |
| cytoskeleton organization (GO: 0007010) | 924 | 100 | 56.37 | + | 1.77 | 1.09E−03 |
| regulation of cell cycle (GO: 0051726) | 1052 | 111 | 64.18 | + | 1.73 | 6.55E−04 |
| positive regulation of nucleobase-containing | 1466 | 153 | 89.44 | + | 1.71 | 2.00E−06 |
| compound metabolic process (GO: 0045935) | | | | | | |
| negative regulation of nitrogen compound | 1871 | 194 | 114.15 | + | 1.7 | 7.16E−09 |
| metabolic process (GO: 0051172) | | | | | | |
| regulation of organelle organization | 1139 | 118 | 69.49 | + | 1.7 | 6.47E−04 |
| (GO: 0033043) | | | | | | |
| nucleobase-containing compound metabolic | 2459 | 254 | 150.03 | + | 1.69 | 2.96E−13 |
| process (GO: 0006139) | | | | | | |
| positive regulation of gene expression | 1538 | 158 | 93.84 | + | 1.68 | 3.11E−06 |
| (GO: 0010628) | | | | | | |
| negative regulation of macromolecule | 2132 | 219 | 130.08 | + | 1.68 | 2.40E−10 |
| metabolic process (GO: 0010605) | | | | | | |
| positive regulation of RNA metabolic process | 1324 | 135 | 80.78 | + | 1.67 | 1.17E−04 |
| (GO: 0051254) | | | | | | |
| negative regulation of cellular metabolic | 2035 | 207 | 124.16 | + | 1.67 | 4.81E−09 |
| process (GO: 0031324) | | | | | | |
| negative regulation of metabolic process | 2328 | 235 | 142.03 | + | 1.65 | 1.01E−10 |
| (GO: 0009892) | | | | | | |
| positive regulation of macromolecule | 1469 | 148 | 89.63 | + | 1.65 | 4.80E−05 |
| biosynthetic process (GO: 0010557) | | | | | | |
| positive regulation of nucleic acid-templated | 1246 | 125 | 76.02 | + | 1.64 | 1.08E−03 |
| transcription (GO: 1903508) | | | | | | |
| positive regulation of RNA biosynthetic | 1247 | 125 | 76.08 | + | 1.64 | 1.10E−03 |
| process (GO: 1902680) | | | | | | |
| heterocycle metabolic process (GO: 0046483) | 2581 | 258 | 157.47 | + | 1.64 | 7.14E−12 |
| cellular aromatic compound metabolic | 2603 | 257 | 158.81 | + | 1.62 | 3.68E−11 |
| process (GO: 0006725) | | | | | | |
| positive regulation of cellular biosynthetic | 1535 | 150 | 93.65 | + | 1.6 | 2.42E−04 |
| process (GO: 0031328) | | | | | | |
| positive regulation of biosynthetic process | 1558 | 152 | 95.06 | + | 1.6 | 2.16E−04 |
| (GO: 0009891) | | | | | | |
| positive regulation of transcription DNA- | 1170 | 114 | 71.38 | + | 1.6 | 1.76E−02 |
| templated (GO: 0045893) | | | | | | |
| cell cycle (GO: 0007049) | 1191 | 116 | 72.66 | + | 1.6 | 1.54E−02 |
| protein-containing complex assembly | 1245 | 12 | 75.96 | + | 1.59 | 9.84E−03 |
| (GO: 0065003) | | | | | | |
| organic cyclic compound metabolic process | 2766 | 267 | 168.76 | + | 1.58 | 1.06E−10 |
| (GO: 1901360) | | | | | | |
| regulation of transcription by RNA polymerase | 1727 | 164 | 105.37 | + | 1.56 | 2.56E−04 |
| II (GO: 0006357) | | | | | | |
| regulation of RNA metabolic process | 2965 | 280 | 180.9 | + | 1.55 | 2.36E−10 |
| (GO: 0051252) | | | | | | |
| positive regulation of macromolecule | 2627 | 248 | 160.28 | + | 1.55 | 1.99E−08 |
| metabolic process (GO: 0010604) | | | | | | |
| cellular nitrogen compound metabolic process | 2979 | 280 | 181.75 | + | 1.54 | 3.74E−10 |
| (GO: 0034641) | | | | | | |
| regulation of nucleobase-containing | 3195 | 299 | 194.93 | + | 1.53 | 5.75E−11 |
| compound metabolic process (GO: 0019219) | | | | | | |
| regulation of gene expression (GO: 0010468) | 3541 | 329 | 216.04 | + | 1.52 | 1.82E−12 |
| organelle organization (GO: 0006996) | 2993 | 276 | 182.61 | + | 1.51 | 7.09E−09 |
| positive regulation of metabolic process | 2842 | 262 | 173.39 | + | 1.51 | 3.39E−08 |
| (GO: 0009893) | | | | | | |
| regulation of macromolecule biosynthetic | 3191 | 291 | 194.69 | + | 1.49 | 3.62E−09 |
| process (GO: 0010556) | | | | | | |
| regulation of cellular macromolecule | 3094 | 282 | 188.77 | + | 1.49 | 1.00E−08 |
| biosynthetic process (GO: 2000112) | | | | | | |
| regulation of cellular biosynthetic process | 3299 | 296 | 201.28 | + | 1.47 | 1.23E−08 |
| (GO: 0031326) | | | | | | |
| positive regulation of nitrogen compound | 2488 | 223 | 151.8 | + | 1.47 | 3.58E−05 |
| metabolic process (GO: 0051173) | | | | | | |
| positive regulation of cellular metabolic | 2605 | 232 | 158.93 | + | 1.46 | 2.89E−05 |
| process (GO: 0031325) | | | | | | |
| regulation of biosynthetic process | 3364 | 299 | 205.24 | + | 1.46 | 3.02E−08 |
| (GO: 0009889) | | | | | | |
| regulation of cellular component organization | 2108 | 187 | 128.61 | + | 1.45 | 2.20E−03 |
| (GO: 0051128) | | | | | | |
| regulation of macromolecule metabolic | 4862 | 431 | 296.64 | + | 1.45 | 6.53E−16 |
| process (GO: 0060255) | | | | | | |
| multi-organism process (GO: 0051704) | 1880 | | 114.7 | + | 1.44 | 3.02E−02 |
| nervous system development (GO: 0007399) | 1871 | 164 | 114.15 | + | 1.44 | 3.69E−02 |
| regulation of nucleic acid-templated | 2742 | 240 | 167.29 | + | 1.43 | 5.69E−05 |
| transcription (GO: 1903506) | | | | | | |
| regulation of RNA biosynthetic process | 2746 | 240 | 167.54 | + | 1.43 | 5.90E−05 |
| (GO: 2001141) | | | | | | |
| regulation of nitrogen compound metabolic | 4589 | 401 | 279.98 | + | 1.43 | 8.87E−13 |
| process (GO: 0051171) | | | | | | |
| negative regulation of biological process | 4184 | 361 | 255.27 | + | 1.41 | 9.82E−10 |
| (GO: 0048519) | | | | | | |
| negative regulation of cellular process | 3727 | 321 | 227.39 | + | 1.41 | 1.12E−07 |
| (GO: 0048523) | | | | | | |
| regulation of cellular metabolic process | 4913 | 423 | 299.75 | + | 1.41 | 5.62E−13 |
| (GO: 0031323) | | | | | | |
| regulation of primary metabolic process | 4740 | 407 | 289.19 | + | 1.41 | 7.25E−12 |
| (GO: 0080090) | | | | | | |
| regulation of transcription DNA-templated | 2692 | 231 | 164.24 | + | 1.41 | 5.92E−04 |
| (GO: 0006355) | | | | | | |
| regulation of metabolic process (GO: 0019222) | 5291 | 454 | 322.81 | + | 1.41 | 1.05E−14 |
| cellular localization (GO: 0051641) | 2128 | 182 | 129.83 | + | 1.4 | 3.57E−02 |
| animal organ development (GO: 0048513) | 2229 | 190 | 135.99 | + | 1.4 | 2.69E−02 |
| regulation of multicellular organismal process | 2341 | 198 | 142.83 | + | 1.39 | 2.12E−02 |
| (GO: 0051239) | | | | | | |
| macromolecule metabolic process | 5167 | 435 | 315.24 | + | 1.38 | 5.59E−12 |
| (GO: 0043170) | | | | | | |
| cell differentiation (GO: 0030154) | 2621 | 219 | 159.91 | + | 1.37 | 1.20E−02 |
| system development (GO: 0048731) | 3224 | 269 | 196.7 | + | 1.37 | 3.76E−04 |
| cellular developmental process (GO: 0048869) | 2708 | 222 | 165.22 | + | 1.34 | 3.80E−02 |
| cellular macromolecule metabolic process | 4346 | 355 | 265.15 | + | 1.34 | 2.88E−06 |
| (GO: 0044260) | | | | | | |
| cellular component organization | 4664 | 377 | 284.56 | + | 1.32 | 1.76E−06 |
| (GO: 0016043) | | | | | | |
| positive regulation of cellular process | 4204 | 339 | 256.49 | + | 1.32 | 5.56E−05 |
| (GO: 0048522) | | | | | | |
| positive regulation of biological process | 4682 | 377 | 285.65 | + | 1.32 | 2.97E−06 |
| (GO: 0048518) | | | | | | |
| cellular component organization or biogenesis | 4845 | 390 | 295.6 | + | 1.32 | 9.72E−07 |
| (GO: 0071840) | | | | | | |
| multicellular organism development | 3670 | 293 | 223.91 | + | 1.31 | 3.18E−03 |
| (GO: 0007275) | | | | | | |
| cellular protein metabolic process | 3181 | 253 | 194.08 | + | 1.3 | 4.84E−02 |
| (GO: 0044267) | | | | | | |
| anatomical structure development | 3949 | 310 | 240.93 | + | 1.29 | 5.65E−03 |
| (GO: 0048856) | | | | | | |
| nitrogen compound metabolic process | 5822 | 454 | 355.21 | + | 1.28 | 3.78E−07 |
| (GO: 0006807) | | | | | | |
| developmental process (GO: 0032502) | 4288 | 332 | 261.62 | + | 1.27 | 5.71E−03 |
| primary metabolic process (GO: 0044238) | 6154 | 471 | 375.46 | + | 1.25 | 2.04E−06 |
| multicellular organismal process | 4652 | 353 | 283.82 | + | 1.24 | 1.40E−02 |
| (GO: 0032501) | | | | | | |
| cellular metabolic process (GO: 0044237) | 6384 | 484 | 389.5 | + | 1.24 | 2.96E−06 |
| organic substance metabolic process | 6420 | 484 | 391.69 | + | 1.24 | 7.44E−06 |
| (GO: 0071704) | | | | | | |
| regulation of cellular process (GO: 0050794) | 7995 | 596 | 487.78 | + | 1.22 | 1.43E−09 |
| regulation of biological process (GO: 0050789) | 8480 | 630 | 517.37 | + | 1.22 | 3.90E−11 |
| metabolic process (GO: 0008152) | 6858 | 497 | 418.41 | + | 1.19 | 1.45E−03 |
| biological regulation (GO: 0065007) | 8970 | 649 | 547.27 | + | 1.19 | 3.77E−09 |
| cellular process (GO: 0009987) | 10694 | 728 | 652.45 | + | 1.12 | 7.00E−06 |
| biological_process (GO: 0008150) | 12719 | 814 | 776 | + | 1.05 | 3.02E−02 |
| Unclassified (UNCLASSIFIED) | 1344 | 44 | 82 | − | 0.54 | 0.00E+00 |

### Example 2: A Majority of FMRP Binding Events in Neurons are Mediated by the Key Co-Factor FXR1P

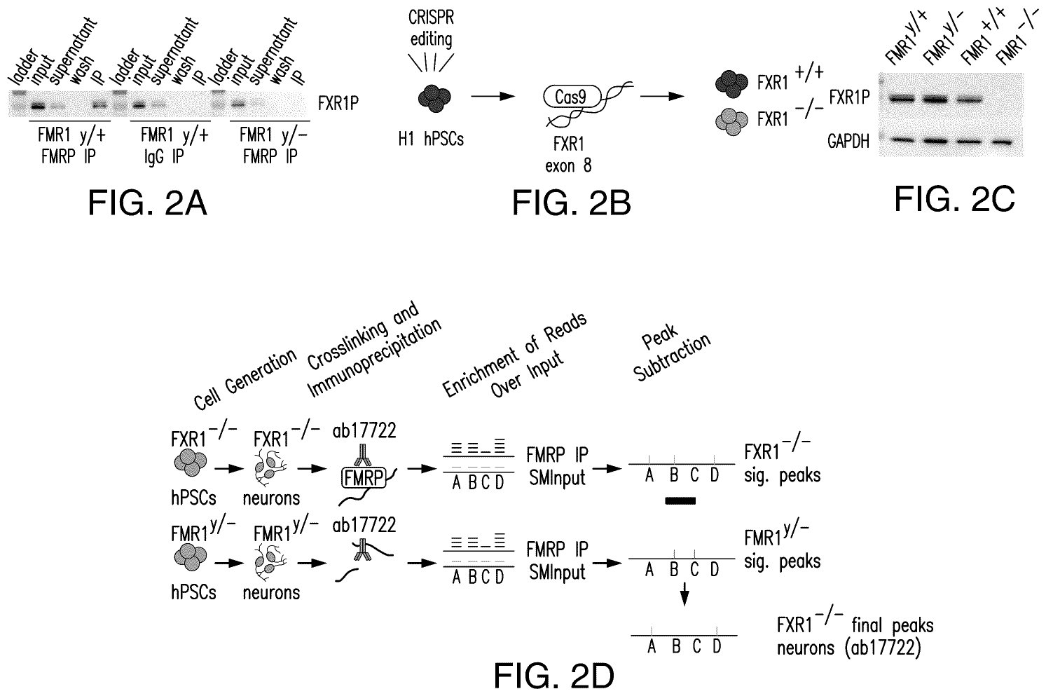

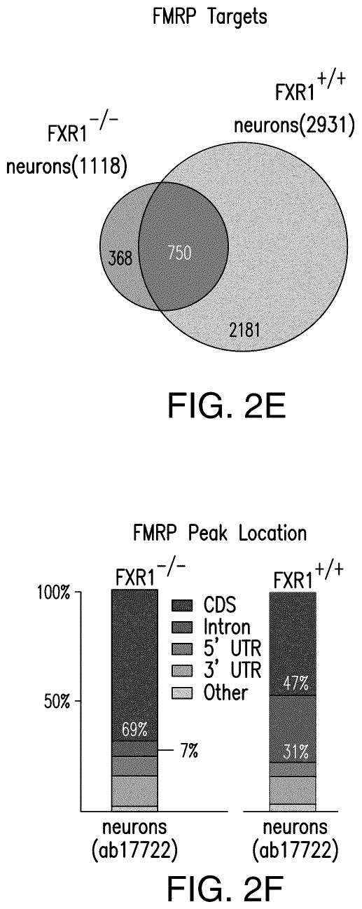

The detection of intron binding events in the datasets described herein (FIG. 1B) indicates that FMRP is capable of binding pre-mRNAs in addition to mature, fully spliced mRNAs. A majority of intronic peaks were distally located (>500 nucleotides from the closest intron-exon junction) and an RNA sequence motif or secondary structure uniquely associated with intron binding was not detected (FIGS. 1I, 1J, 1K), suggesting that other features drive FMRP target recognition in these datasets. While little is known about the role of FMRP in the regulation of pre-mRNA targets, one of the few known nuclear co-factors of FMRP is its autosomal paralog FXR1P⁵², encoded by the Fragile X mental retardation syndrome-related 1 (FXR1) gene. Given their propensity to heterodimerize (FIG. 2A), it was contemplated whether FMRP binding events, including pre-mRNA binding events, were dependent or independent of its co-factor FXR1P. To this end, an FXR1^{-/-} hPSC line was generated using CRISPR-Cas9 (FIG. 2B), isogenic with the FMR1^{y/+} and FMR1^{y/-} hPSC lines previously used for FMRP eCLIP-seq, and confirmed expected loss of FXR1P expression (FIG. 2C). FXR1^{-/-} hPSCs were then differentiated into neurons and FMRP eCLIP-seq was performed to probe FMRP targets in the absence of FXR1P, again using FMR1^{y/+} peak subtraction (FIG. 2D, Table 3). This allowed comparison of FMRP targets in FXR1^{+/+} neurons (FIGS. 2A-2F) with FMRP targets in FXR1^{-/-} neurons (FIG. 2D). Notably, FXR1P was required for three-quarters of FMRP binding events, as 74.4% of FMRP targets were lost in FXR1^{-/-} neurons and 25.6% of FMRP targets were retained in FXR1^{-/-} neurons (FIG. 2E). A small number of targets were gained by FMRP upon FXR1P loss (FIG. 2E), which could be due to changes in the underling transcriptional landscape. Importantly, FMRP intronic peaks were substantially reduced upon FXR1P loss (FIG. 2F). These data indicate that FXR1P is a critical co-factor for a majority of FMRP binding events in neurons, including a majority of pre-mRNA binding events.

TABLE 3
Summary of final FMRP eCLIP-seq dataset in
FXR1−/− neurons following FMR1y/− peak subtraction

| Cell | | Significant | | | % |
|---|---|---|---|---|---|
| Type | Antibody | Peaks | Genes | Cell Input | Subtracted |
| neurons | ab17722 | 3120 | 1118 | 7.0E+07 | 33.5 |

### Example 3: FMRP Targets are Enriched for Key Neurodevelopmental Disease Associated Genes

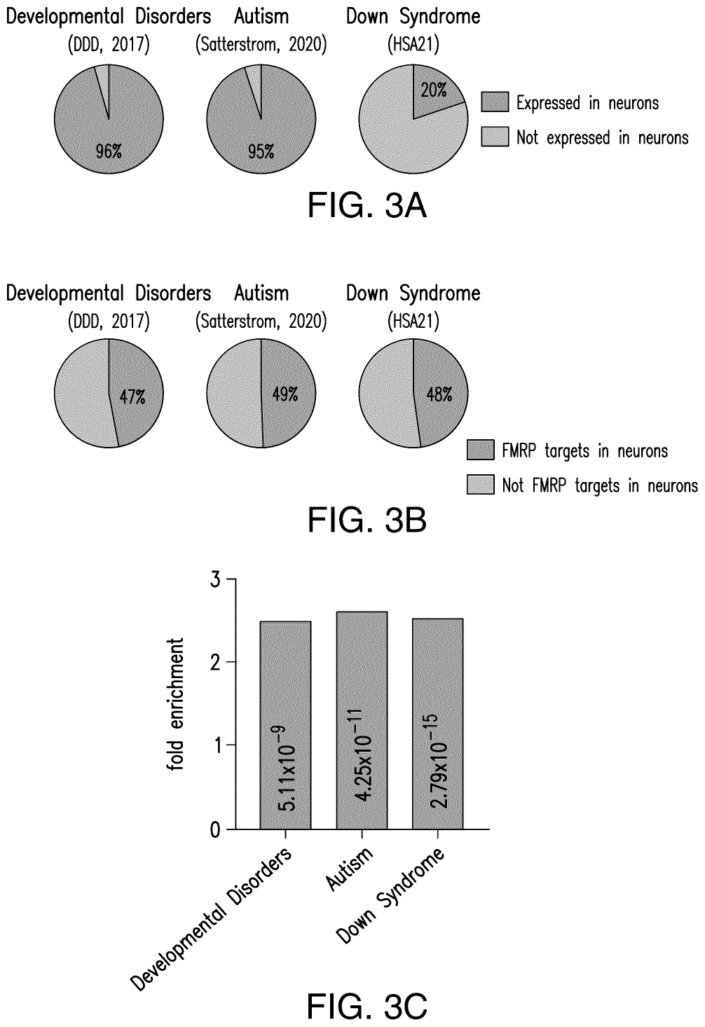

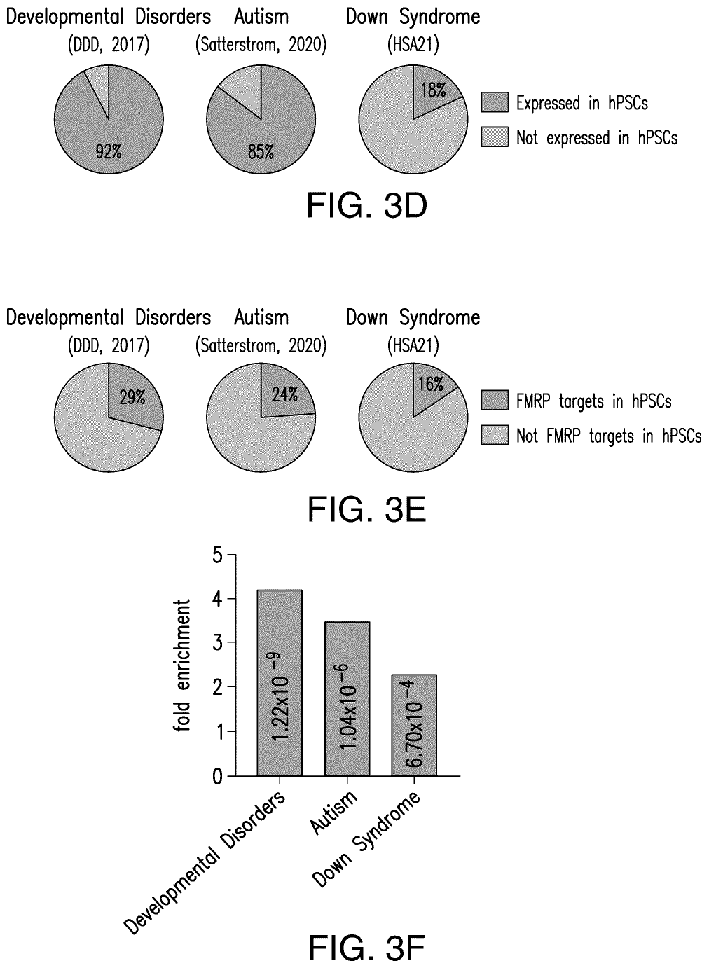

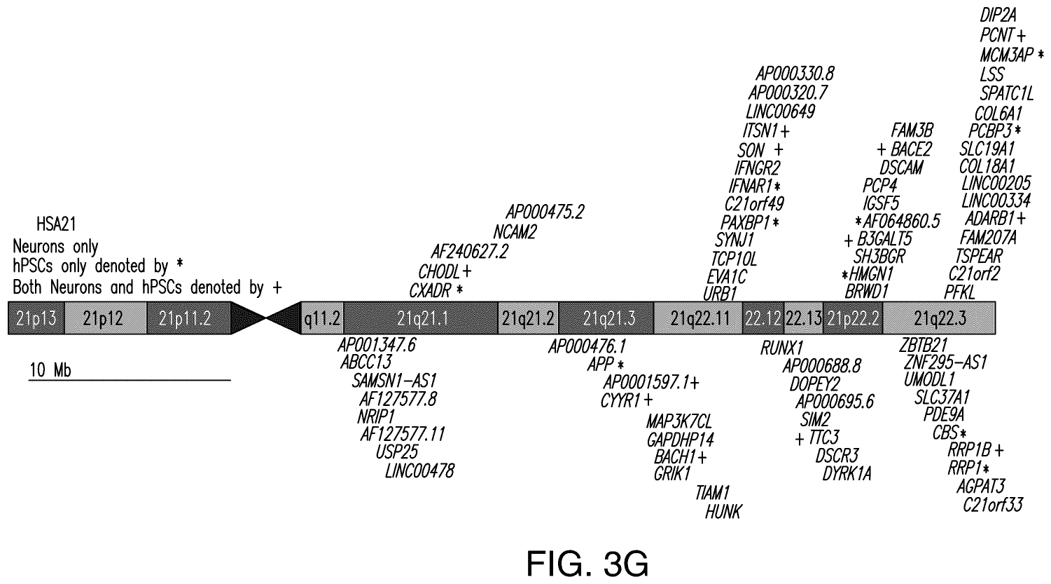

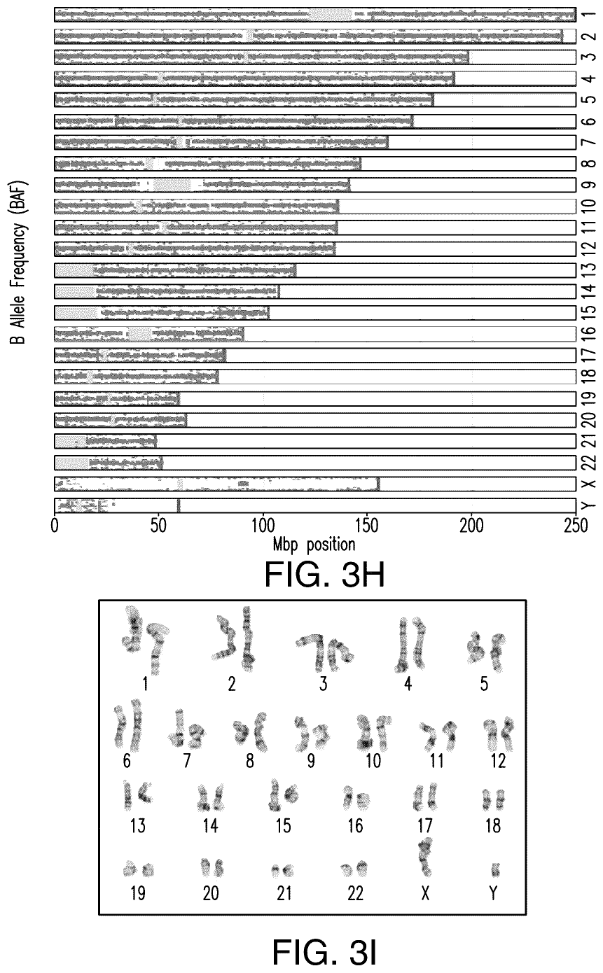

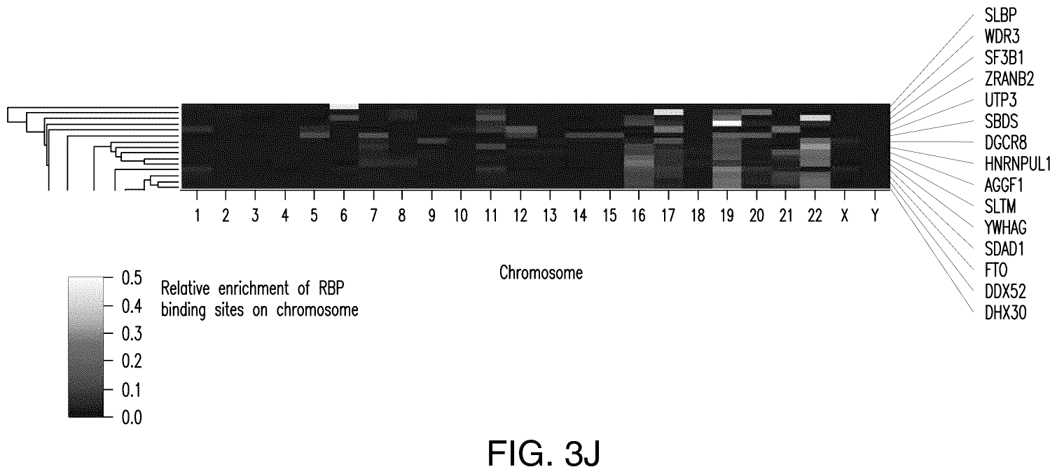

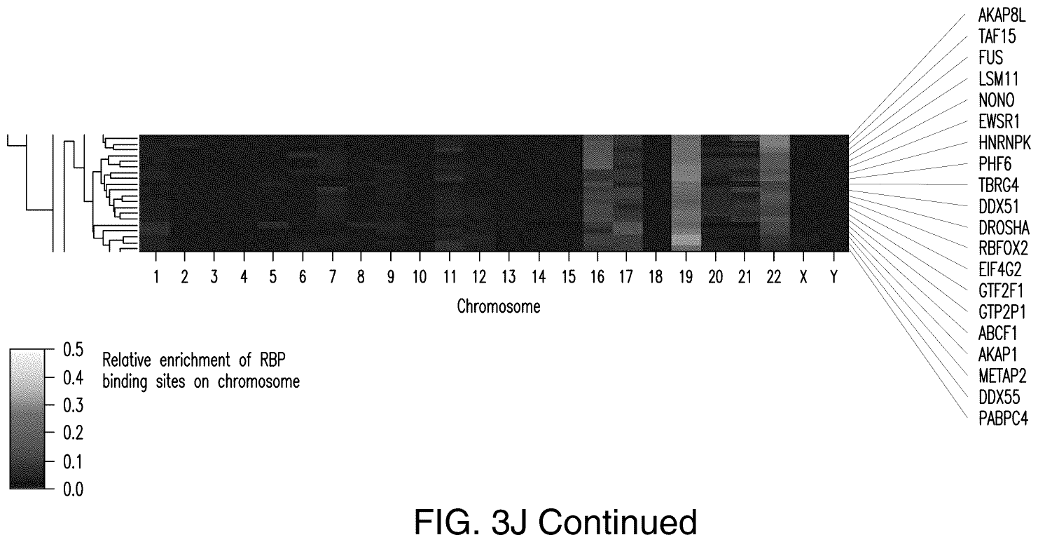

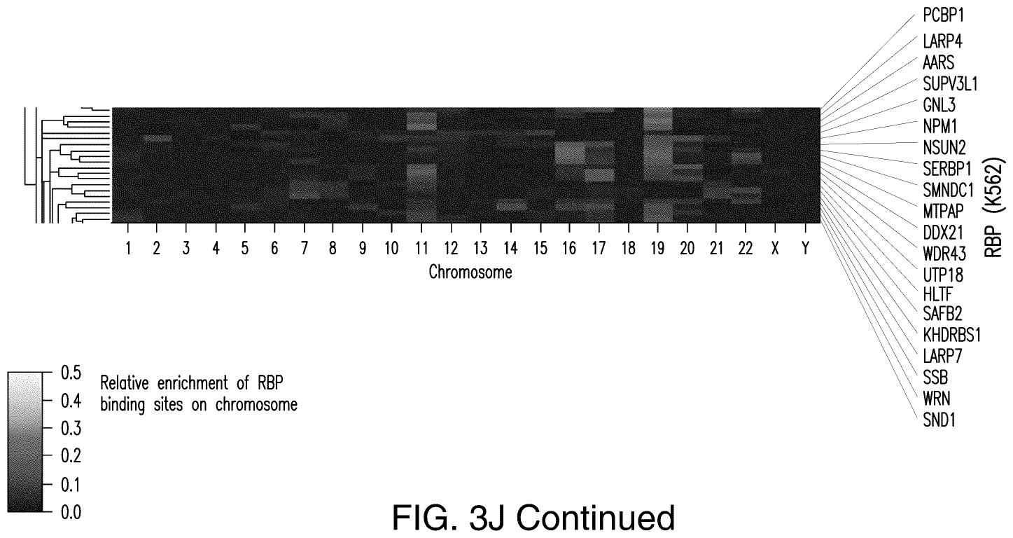

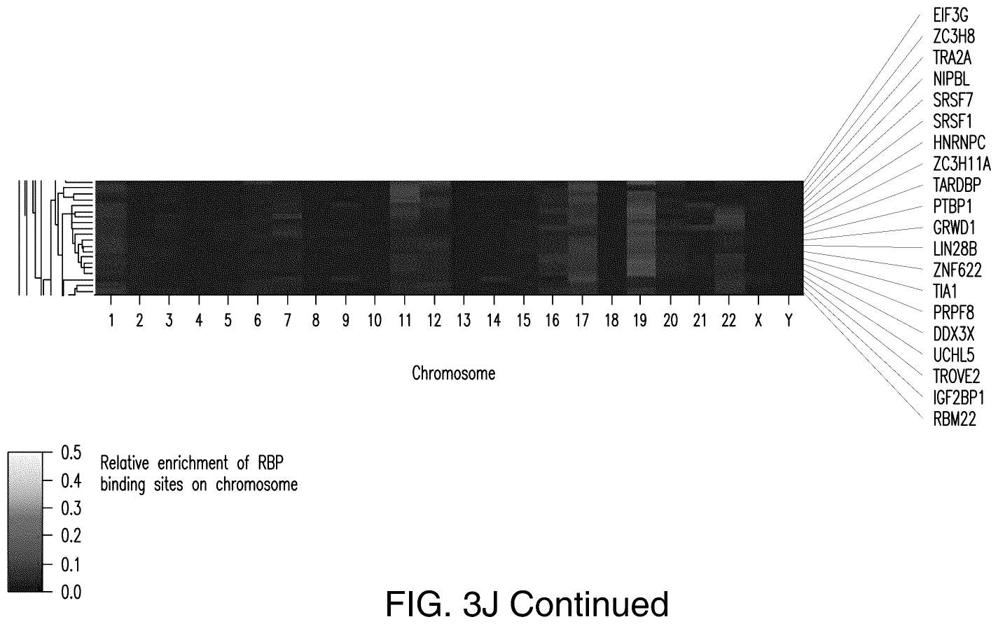

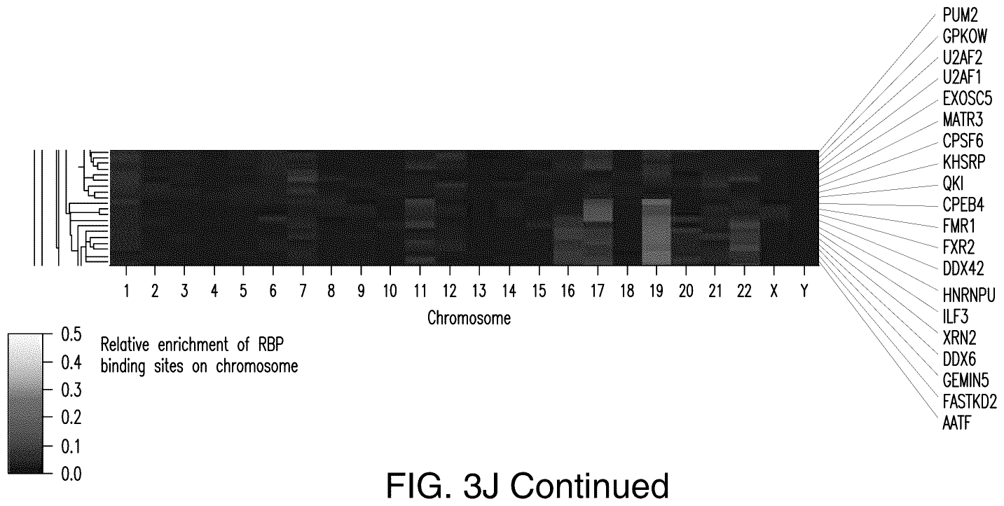

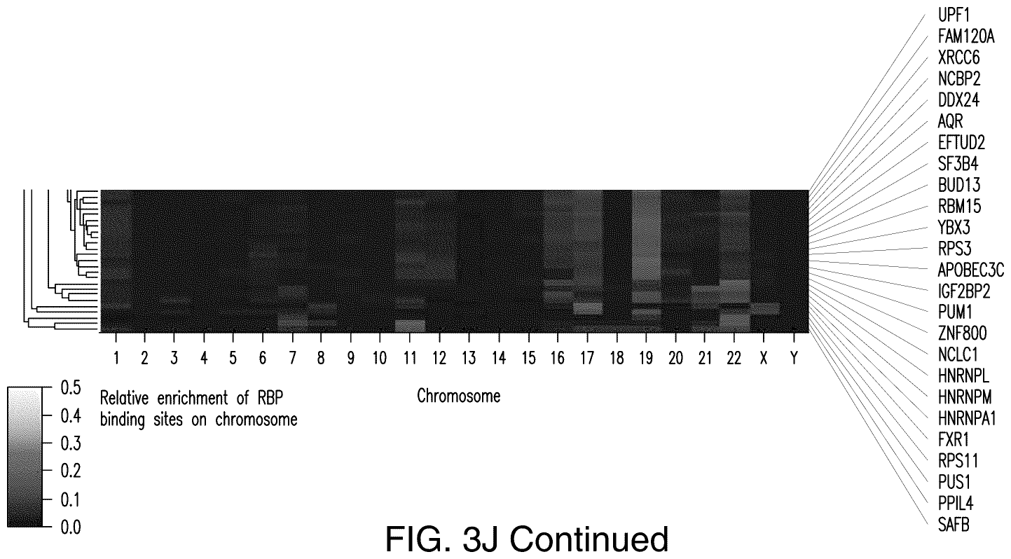

It was next assessed whether FMRP targets in human neurons would show significant enrichment for neurodevelopmental disease associated genes (FIGS. 3A-3C), as previously shown (Iossifov, I. et al., Nature 515, 216-21 (2014), Sanders, S. J. et al., Neuron 87, 1215-33 (2015), Schizophrenia Working Group of the Psychiatric Genomics, Nature 511, 421-27 (2014)) for FMRP targets from mouse brain. Notably, 96% of genes implicated in developmental disorders and 95% of high-confidence autism susceptibility genes identified through exome sequencing were expressed in human neurons (FIG. 3A), consistent with studies indicating that excitatory neurons are enriched for the expression of autism risk genes. Moreover, nearly half of all expressed genes from each disease dataset were FMRP targets (FIG. 3B); FMRP targets in human neurons were 2.47-fold enriched (p=5.11×10⁻⁹) for genes implicated in developmental disorders and 2.59-fold enriched (p=4.25×10⁻¹¹) for autism susceptibility genes (FIG. 3C). These data are consistent with previous studies showing that FMRP targets are enriched for genes independently implicated in neurodevelopmental diseases, and expand upon the specific overlapping targets with relevance to the developing human brain. These data also support the use of human in vitro derived excitatory neurons for study of neurodevelopmental disease mechanisms.

Unexpectedly, a substantial number of FMRP targets transcribed from HSA21 and implicated in DS disease biology were also noted, such as APP, DYRK1A, NCAM2, PCP4, DSCAM, BACE2, BACH1 and RUNX1. While 20% of genes encoded on HSA21 were expressed in human neurons (FIG. 3A), nearly half of those expressed genes were FMRP targets (FIG. 3B). Specifically, FMRP targets in human neurons were 2.51-fold enriched (p=2.79×10⁻¹⁵) for HSA21-encoded transcripts, closely paralleling results obtained for developmental disorders and autism (FIG. 3C). As expected, fewer disease implicated genes were expressed in hPSCs although FMRP targets were still enriched for genes implicated in developmental disorders, autism and Down syndrome in this cell type (FIGS. 3D-3F).

These results raise the novel possibility that FXS and DS may share some underlying molecular mechanisms during early brain development as previously proposed for FXS and other neurodevelopmental diseases. FMRP RNA targets were distributed across the q-arm of HSA21 and did not cluster in a specific cytoband (FIG. 3G). Cells used for eCLIP-seq did not have chromosomal aberrations by G-band analysis or high-density SNP array (FIGS. 3H, 3I). Additionally, existing eCLIP-seq datasets for 120 RNA binding proteins in K562 leukemic cells available through the ENCODE consortium (E.P. Consortium, Nature 489, 57-74 (2012)) were leveraged to confirm a lack of widespread HSA21 binding events in eCLIP-seq datasets generally (FIG. 3J).

Collectively, these data indicate that FMRP binds a significant number of neurodevelopmental disease-associated genes in human neurons. Moreover, the discovery described herein that FMRP targets are enriched for genes transcribed from HSA21, provides a potential molecular link between FXS and DS.

### Example 4: FXS and DS Converge on Shared Transcriptional Perturbations

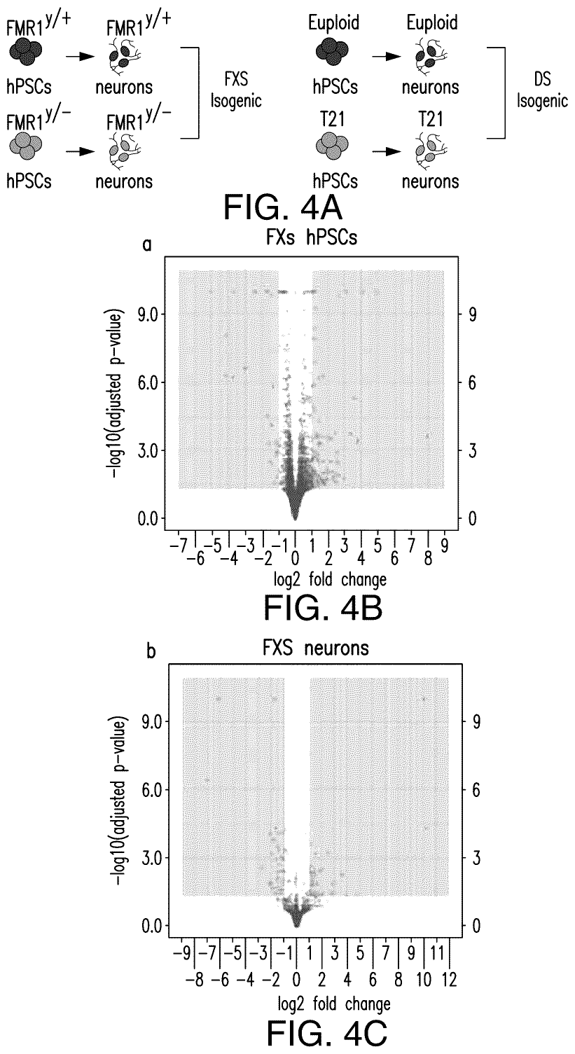

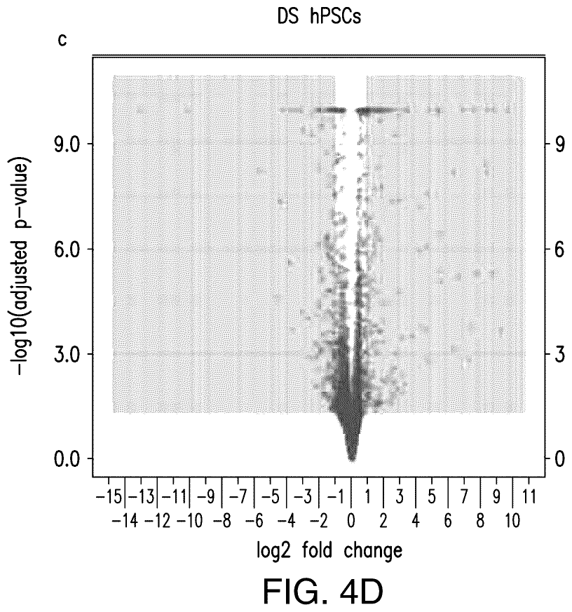

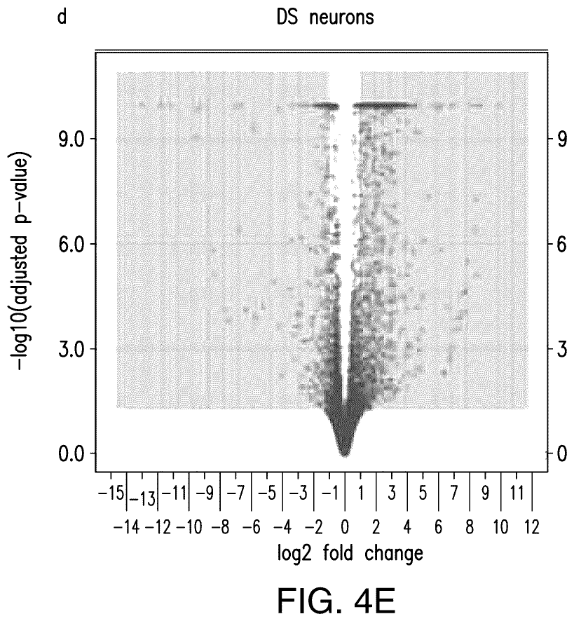

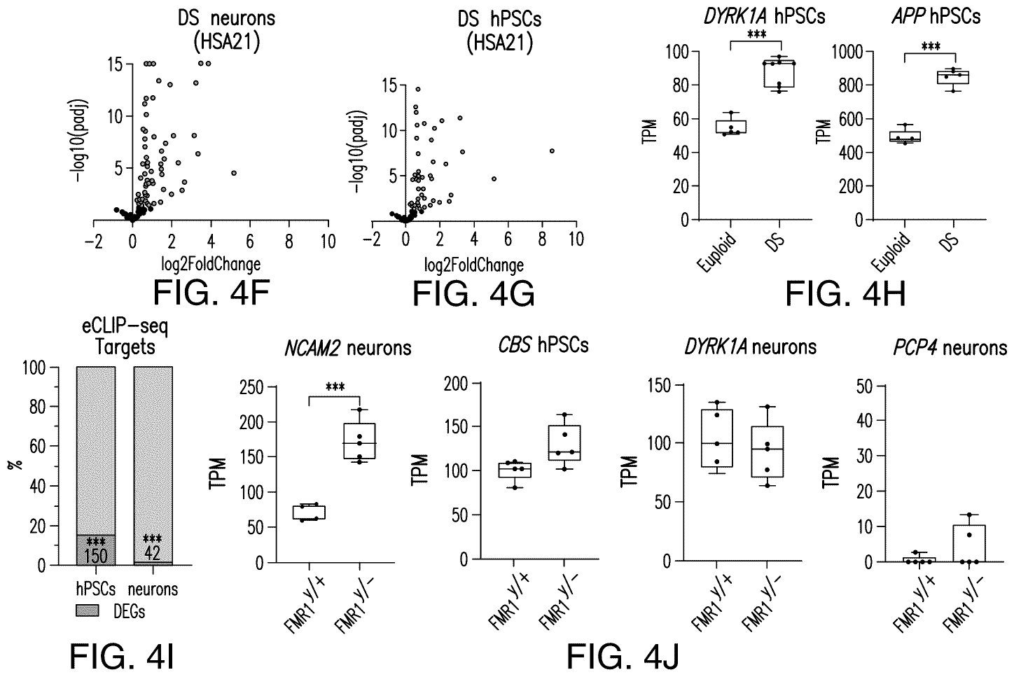

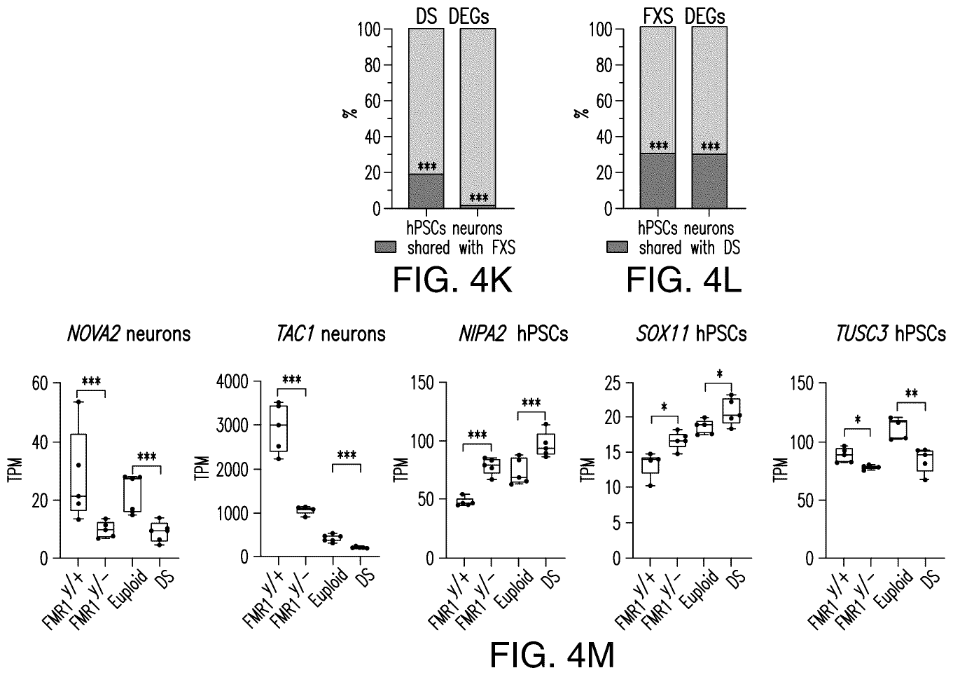

The above FMRP target analyses support a novel model whereby FXS and DS may result in the perturbation of a significant number of the same genes through different mechanisms. However, the data described herein combined with previous studies also support complex roles for FMRP in different stages of RNA processing (Verheij, C. et al., Nature 363, 722-724 (1993); Zhou, L. T. et al., Neuroscience 349, 64-75 (2017); Tran, S. S. et al., Nature neuroscience 22, 25-36 (2019); Alpatov, R. et al., Cell 157, 869-881 (2014); Chakraborty, A. et al., Fragile X Mental Retardation Protein regulates R-loop formation and prevents global chromosome fragility. bioRxiv (2019); Kim, M. et al., Molecular and cellular biology 29, 214-228 (2009); D′Souza, M. N. et al., iScience 9, 399-411 (2018); Edens, B. M. et al., Cell reports 28, 845-854 e845 (2019); Dury, A. Y. et al., PLoS Genet 9, e1003890 (2013); Taha, M. S. et al., PloS one 9, e91465 (2014)), making it challenging to predict how FMRP loss would impact the expression or function of any individual target. Therefore, global transcriptional perturbations were first quantified in FXS and DS cellular models to broadly assess molecular convergence. Specifically, RNA-seq was performed using an isogenic pair of DS patient and euploid control iPSC lines (generated from a mosaic patient fibroblast line) (Weick, J. P. et al., PNAS, 110, 9962-9967 (2013)), as well as the isogenic FMR1^{y/+} and FMR1^{y/-} lines (Susco, S. G. et al., FMR1 loss results in early changes to intrinsic membrane excitability in human cellular models. bioRxiv (2020)) examined transcriptional changes in both pluripotent stem cells and neurons (FIGS. 4A-4F).

Within each cell type and disease state comparison, roughly equal numbers of genes were observed being significantly upregulated and downregulated (FIGS. 4B-4F, Table 4). As expected, an extra copy of HSA21 led to significant upregulation of HSA21-encoded transcripts in both hPSCs (FIGS. 4G, 4H) and neurons (FIGS. 4B-4F). Cross-referencing the FMRP eCLIP-seq data with genes differentially expressed following FMR1 loss revealed that a small but significant fraction of FMRP targets were differentially expressed at the transcript level following FMR1 loss (FIG. 41). For example, the FMRP target NCAM2 was significantly upregulated at the transcript level following FMR1 loss (p=0.000154), while the FMRP targets CBS, DYRK1A and PCP4 remained unchanged (FIG. 4J).

TABLE 4
Summary of RNA-seq datasets

| Cell | Isogenic | Biological | Total | Upregulated | (Downregulated |
|---|---|---|---|---|---|
| type | Pair | Replicates | DEGs | DEGs | DEGs |
| hPSCs | FXS | 5 | 1614 | 819 | 795 |
| neurons | FXS | 5 | 130 | 62 | 68 |
| hPSCs | DS | 5 | 2578 | 1346 | 1232 |
| neurons | DS | 5 | 2991 | 1648 | 1343 |

These data indicate that FMRP modulates RNA expression or stability of a minority of its targets, and thus a majority of genes differentially expressed following FMRP loss are likely the result of indirect, secondary changes as opposed to direct FMRP binding. Importantly, significant overlap was found in differential gene expression patterns between FXS and DS hPSC (p=3.19×10⁻³³) and neuron (p=0.00495) models (FIGS. 4K, 4L). In DS models, although thousands of genes were significantly differentially expressed, 18.5% of transcriptional changes in hPSCs were shared with FXS (FIG. 4K). In FXS models, 29.5% of significant DEGs in hPSCs and 29.2% of significant DEGs in neurons were also significantly perturbed in DS (FIG. 4L). For example, NOVA2 and TAC1 transcript expression levels were significantly downregulated in neurons derived from both FXS and DS models compared to controls (FIG. 4M). Similarly, in hPSCs, NIP2A and SOX11 transcript expression levels were significantly upregulated while TUSC3 transcript expression levels were significantly downregulated in both FXS and DS models compared to controls (FIG. 4M).

NOVA2, NIPA2, SOX11 and TUSC3 are notable for their independent associations with neurodevelopmental diseases. All four genes were coordinately misregulated at the transcript level in FXS and DS models; NOVA2 was an FMRP target from HSA19 while NIPA2, SOX11 and TUSC3 were neither FMRP targets nor localized to HSA21, suggesting that FXS and DS can converge on shared transcriptional perturbations through direct or indirect mechanisms.

### Example 5: FSX and DS Converge on Shared Proteomic Perturbations

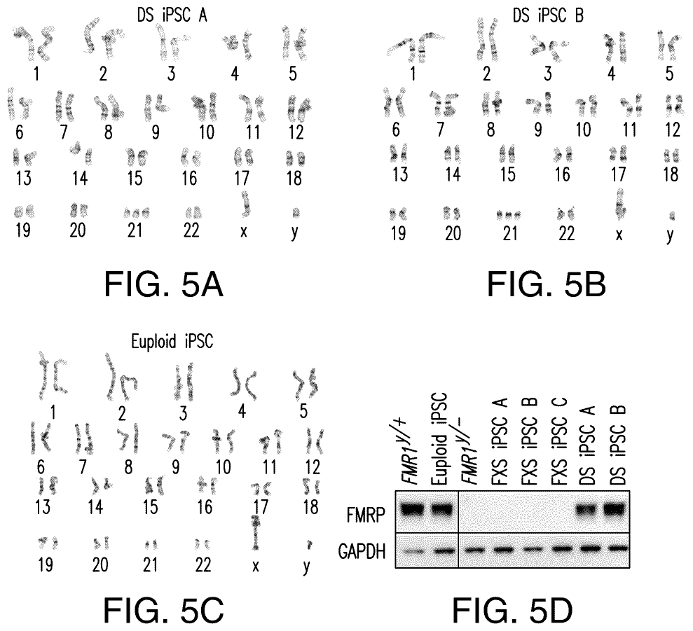

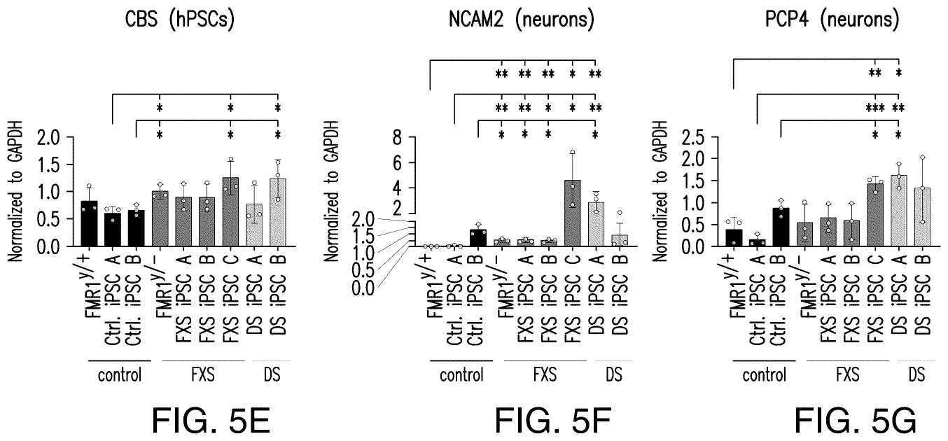

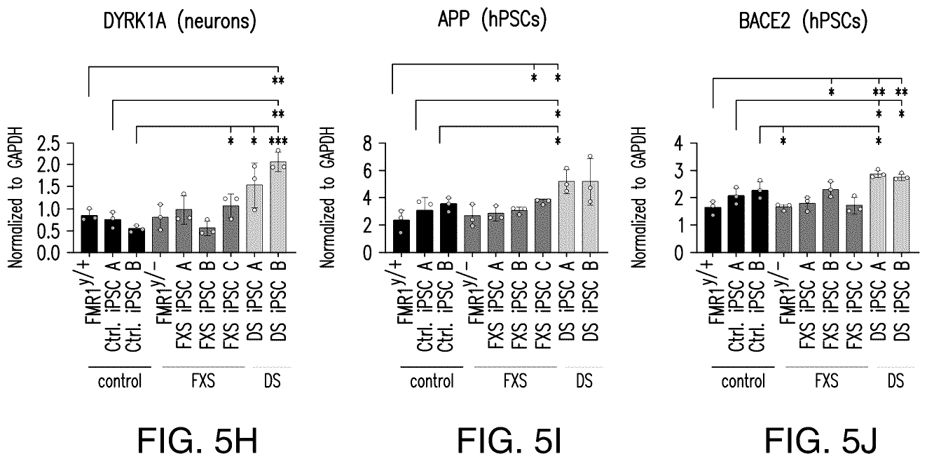

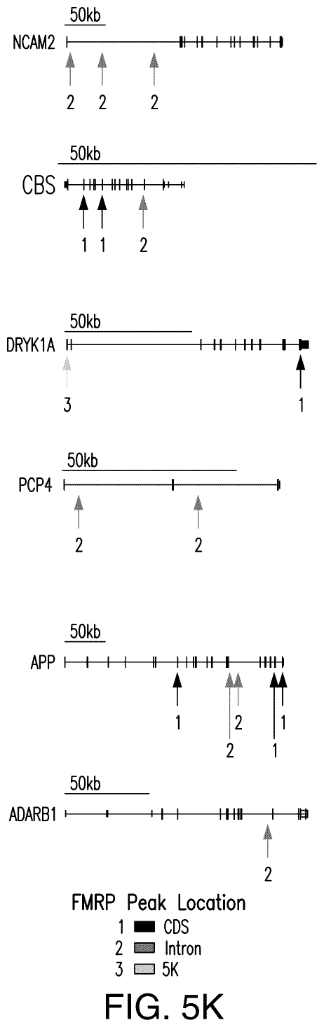

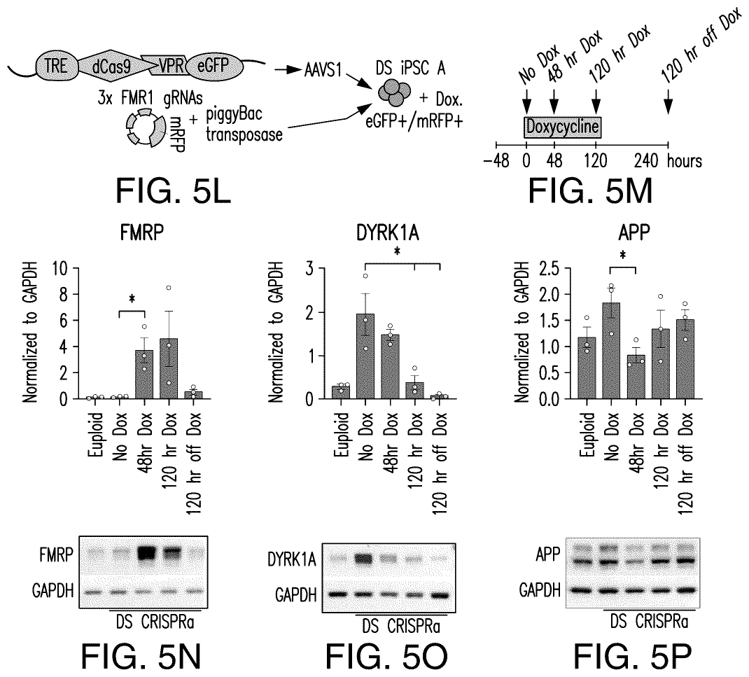

While studies using human fetal and adult post-mortem brain tissue consistently report an overall increase in expression of genes transcribed from HSA21 in DS patients compared to euploid controls, a limited number of genes have been assessed at the protein level. Therefore, DS and FXS patient and control iPSC lines were generated or acquired and protein expression levels of a set of individual HSA21-encoded FMRP targets was assessed across multiple genetic backgrounds, focusing on targets implicated in specific aspects of DS disease biology. Specifically, a set of three iPSCs reprogrammed from FXS patient fibroblast lines were used and two DS patient iPSC lines were acquired plus a euploid iPSC control in addition to the isogenic FMR1^{y/+} and FMR1^{y/-} CRISPR engineered hPSC lines (FIGS. 5A-5D). Protein expression levels were then assayed for six FMRP targets encoded on HSA21: NCAM2 (cell adhesion molecule), CBS (enzyme in the transsulfuration pathway), DYRK1A (tyrosine kinase), PCP4 (calmodulin binding protein), APP (amyloid precursor protein) and ADARB1 (RNA editing enzyme) (FIGS. 5E-5J). Strikingly, NCAM2 protein expression levels were significantly upregulated in both FXS and DS cell lines compared to controls (FIG. 5E). This was true for both isogenic FXS and DS models as well as genetically diverse FXS and DS patient cell lines with a large magnitude of effect (FIG. 5E). While NCAM2 overexpression has been reported to mimic a subset of the aberrant neuronal development phenotypes seen in DS mouse models, it has not previously been studied in the context of FXS.

CBS expression was also significantly upregulated in individual FXS and DS patient lines, but significant differences were not observed for all genetic backgrounds (FIG. 5F). CBS is reportedly upregulated in DS patient brain tissue compared to controls and upregulated CBS is necessary and sufficient for induction of cognitive phenotypes. Similar to NCAM2, CBS has also not been studied in the context of FXS. Several targets were observed to be upregulated in DS but not FXS or that remained unchanged across disease states.

DYRK1A was significantly upregulated in both DS patient lines consistent with previous studies showing upregulated DYRK1A expression in post-mortem brain tissue from DS patients, but remained unchanged across all FXS cell lines (FIG. 5G). Mutations in DYRK1A are independently associated with autism and intellectual disability and inhibition of DYRK1A has been shown to partially rescue DS phenotypes in cellular models. DYRK1A is also implicated in DS Alzheimer's Disease (AD) pathogenesis.

In the case of PCP4, significant upregulation was observed in one DS patient line compared with its euploid isogenic control, but no change was observed in the other DS patient line or the FXS lines (FIG. 5H). Increased levels of PCP4 were previously shown to induce differentiation defects and ciliopathies in mice paralleling some of those observed in DS during fetal development.

Interestingly, increased APP expression is thought to drive increased risk for AD seen in DS patients and APP is also reportedly upregulated in DS fetal brain tissue compared to controls. While observed a trend toward upregulated APP expression was observed in both DS patient cell lines, this did not reach statistical significance (FIG. 5I).

For ADARB1, significant changes in protein expression levels were not observed in either FXS or DS patient cells, but unlike APP, expression levels of ADARB1 were highly consistent across different genetic backgrounds (FIG. 5J). These results are consistent with published studies of DS patient brain tissue, which report ADARB1 protein expression to be unchanged compared to controls.

It is critical to note that there is significant heterogeneity reported among individual FXS and DS patients for nearly all identified phenotypes. For example, even for FMRP's canonical role in protein synthesis, analyses of patient fibroblasts revealed significant variability, with some patients' fibroblasts showing increased protein synthesis and others showing no change. Indeed, the results for some targets were consistent across genetic backgrounds within each disease state (e.g., NCAM2, DYRK1A, ADARB1), and for other targets, more variability was observed depending on genetic background (e.g., CBS, PCP4, APP). As expected, with the exception of NCAM2, protein-level effect sizes were generally modest for both FXS and DS.

With regard to the two targets upregulated at the protein level in FXS cell lines, NCAM2 and PCP4, the FMRP binding locations from the eCLIP-seq dataset were examined as well as transcript expression levels from our RNA-seq data. Interestingly, while upregulated NCAM2 protein expression levels in FXS were observed (FIG. 5E), FMRP only binding events in introns of this gene were detected (FIG. 5K). Moreover, NCAM2 was significantly increased at the transcript level following FMRP loss (FIG. 4J), suggesting that FMRP impacts either NCAM2 expression or stability, which then leads to protein level changes.

CBS was significantly upregulated in an FXS patient line (FIG. 5F) but not in the isogenic FMR1^{y/+} and FMR1^{y/+} lines at the transcript level (FIG. 4J) or at the protein level (FIG. 5E). In contrast to NCAM2, FMRP bound CBS in both CDS and intronic regions (FIG. 5K) leaving open multiple possible mechanisms of CBS gene regulation in the FXS patient line. Collectively, these data indicate that FMRP modulates protein expression levels for a subset of targets transcribed from HSA21, and places select targets at the interface between FXS and DS biology.

### Example 6: Acute Upregulation of Endogenous FMRP in DS Patient Cells Normalizes Protein Expression Levels of Key HSA21-Encoded Targets

Although some HSA21-encoded FMRP targets like DYRK1A were significantly upregulated in DS but not FXS (FIG. 5G), it was reasoned that increasing FMRP dosage in the context of DS could potentially further modulate target expression. Therefore, an inducible CRISPR activation (CRISPRa) construct was stably introduced into the AAVS1 safe-harbor locus of a DS patient iPSC line, along with a multiplexed piggyBac integration plasmid encoding three FMR1 activating gRNAs (DS-CRISPRa) (FIG. 5L). As expected, doxycycline induction of the DS-CRISPRa cell line led to efficient and transient upregulation of FMRP protein expression (FIG. 5M). Strikingly, acute upregulation of endogenous FMRP was sufficient to significantly decrease protein expression levels of DYRK1A in the DS-CRISPRa line (FIG. 5N). Here, transient FMRP upregulation led to a sustained reduction in protein expression that lasted beyond withdrawal of doxycycline and normalization of FMRP levels (FIG. 5N).

Although APP was not significantly upregulated in DS patient lines (FIG. 5I), a trend was observedtoward increased APP expression; therefore, APP expression levels following FMRP upregulation were also probed in the DS-CRISPRa cell line. Again, significant reduction was achieved in APP protein expression, but in this case, APP levels were more transiently reduced and recovered by the 120-hour time-point (FIG. 5O). FMRP bound DYRK1A in both CDS and non-coding exon regions and APP in both CDS and intron regions (FIG. 5C), again leaving open multiple possible mechanisms of gene regulation. Importantly, these data provide proof-of-concept that transiently increasing endogenous FMRP in the context of DS is sufficient to significantly modulate key HSA21-encoded targets.

Given that HSA21-encoded transcripts are increased in abundance in DS due to the extra copy of the chromosome, and FMRP regulates a large number of these transcripts, it was hypothesized that increasing FMRP dosage in the context of DS would modulate expression of HSA21-encoded targets. Using CRISPRa in two independent DS patient iPSC lines, endogenous FMRP was acutely upregulated and normalized protein expression levels of multiple FMRP targets, including APP (amyloid-B precursor protein) and DYRK1A. Increased APP expression is associated with the highly elevated rates of Alzheimer's disease in DS patients (DS affects 1 in 700 and by age 40, almost all patients have amyloid plaques). DYRK1A is independently implicated in autism and intellectual disability.

It is critical to emphasize that existing knowledge of molecular mechanisms underlying FXS and DS have failed to translate into effective therapeutic strategies. Indeed, there are currently no effective treatments for FXS or DS, which may be due in part to the large number of genes thought to be simultaneously and subtly disrupted in each disease. By defining high-confidence FMRP targets for the first time in two physiologically relevant human cell types, novel molecular overlap between FXS and DS was uncovered, paralleling results for FXS and other neurodevelopmental diseases. Without wishing to be bound by theory, while many gene targets and patient phenotypes do not overlap in FXS and DS, it is proposed that genes misregulated in both diseases are candidates for contributing to the shared aspects of disease pathology, which includes intellectual disability, increased rates of autism, and increased rates of other mental health disorders compared with the general population. The finding of shared molecular perturbations, combined with data showing that acutely upregulating endogenous FMRP can modulate the key HSA21-encoded targets DYRK1A and APP, suggests there may be opportunities for shared therapeutic intervention in FXS and DS.

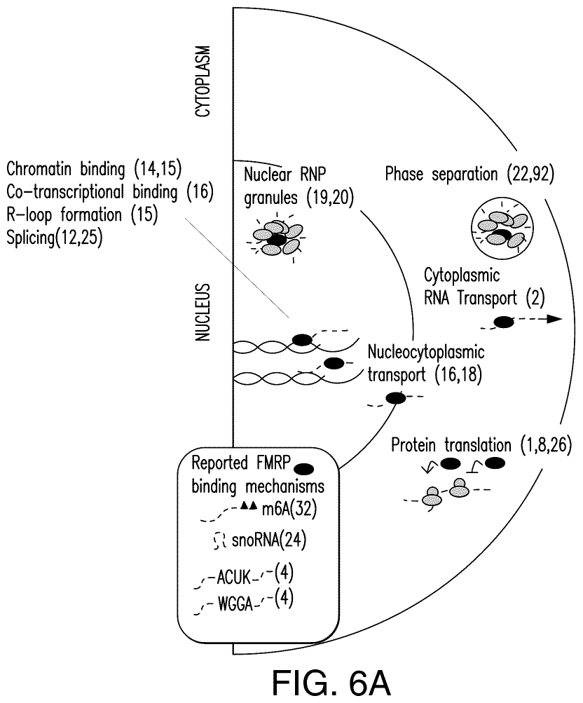

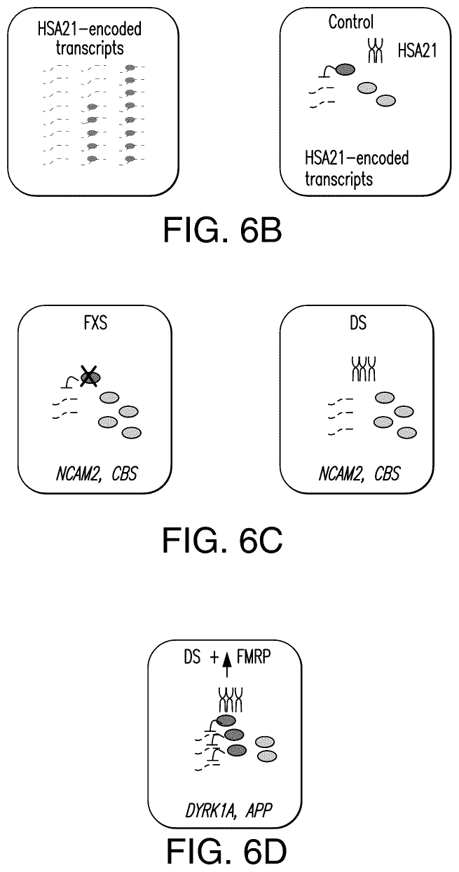

It is likely there is no single mechanism of action for FMRP in a given cell type and cell state. The analyses presented herein highlight a more complex picture of gene regulation (FIGS. 6A-6D). For many targets, FMRP binding events were detected across multiple regions of a given transcript (i.e., both intronic and CDS regions), and it is challenging to ascertain whether FMRP is binding a single transcript molecule in multiple regions at the same time or interacting with different transcript molecules at different stages of processing. While FXS is canonically associated with increased protein expression and DS with increased gene dosage through an extra copy of a chromosome, it is reductionist to assume that all FMRP targets on HSA21 are coordinately upregulated in FXS and DS.

Without wishing to be bound by theory, in cases where FMRP targets are increased at the protein level in FXS but also bound as pre-mRNAs, it is speculated that FMRP may reduce the kinetics of post-transcriptional processing in the nucleus, and upon FMRP loss, transcripts are more readily processed and exported, leading to increased association with translational machinery. However, as discussed above, FMRP has previously been associated with diverse processes including transcriptional regulation, RNA splicing, and nuclear RNA export. Loss of FMRP followed by perturbation of any of these processes could impact downstream protein expression levels of individual targets. Even for fully mature mRNA targets, FMRP can phase-separate through its RGG domain and this sub-compartmentalization has been shown to impact deadenylation to inhibit translation in the cytoplasm, distinct from FMRP's canonical role in direct translational regulation. As FMRP localization and function likely change across developmental trajectories, and given that the analyses described herein are elucidating for early development, the analyses and studies described herein provide a basis for studies that may also elucidate FMRP's role in the developing versus the aging human brain.

While much remains to be learned about the mechanisms of action of FMRP for each individual RNA target, the model described herein raises the novel possibility that future therapeutic strategies aimed at DS may be relevant for FXS, or vice versa, based on convergent target biology. For example, clinical trials have attempted to normalize DYRK1A with the goal of improving cognitive function in DS patients, and both DYRK1A and APP are strongly implicated in the development of AD in DS patients. Given that increasing FMRP from the endogenous locus alone was sufficient to reduce the expression levels of DYRK1A and APP in DS patient cells, methods to increase FMRP as potential therapeutic strategies in FXS could also have utility for DS patients or other patients with DYRK1A or APP over-expression.

Collectively, these data provide the first evidence for direct mechanistic overlap between FXS and DS and identify key genes at the interface of two of the most common genetic causes of intellectual disability and autism.

### Example 7: Genes Associated with One or More Neurodevelopmental or Neurodegenerative Diseases or Disorders

Based on a combination of RNAseq, CLIPseq and Western blot analyses in control and patient derived cells (e.g., hPSCs or neurons), it was discovered that genes associated with one neurodevelopmental or neurodegenerative disease or disorder were also associated with another neurodevelopmental or neurodegenerative disease or disorder. Thus, the studies identified genes whose aberrant activity (e.g., an increase or decrease in activity) overlapped with different neurodevelopmental or neurodegenerative diseases or disorders. In some cases, a gene known in the context of one disease or disorder was also surprisingly found to be associated with another disease or disorder. In some cases, the neurodevelopmental or neurodegenerative disease or disorder associated with a given gene is both Fragile X syndrome (FXS) and Down syndrome (DS). In some cases, the neurodevelopmental or neurodegenerative disease or disorder associated with a given gene is Fragile X syndrome (FXS). In some cases, the neurodevelopmental or neurodegenerative disease or disorder associated with a given gene is Down syndrome (DS).

Table 5 (Appendix) presents a list of genes (polynucleotides) and their association with one or more diseases as determined based on the studies carried out herein. In addition, Table 5 indicates how the genes (polynucleotides) change across various datasets. In brief, a gene name in a column means that gene was identified in a given dataset. A value in a column is shown as the log2fold change (for RNAseq). Values that signify an increase or enhancement (e.g., an increase or enhancement in expression, expression level, amount, or activity) are shown in solid line cells (positive numerical values) in Table 5. Values that signify a decrease or reduction (e.g., an decrease or reduction in expression, expression level, amount, or activity) are shown in dotted line cells (negative numerical values) in Table 5. A positive numerical value (solid line cells) in the table indicates that the function or activity of the indicated gene (polynucleotide) is expected to be activated in a dataset, while a negative numerical value (dotted line cells) indicates that the function or activity of the indicated gene (polynucleotide) is expected to be inhibited in a dataset. In some cases, a specific likelihood of activation or inhibition is not presented.

In particular, as presented herein, Table 5 includes 14 columns, namely, Columns A-N, designated from left to right. Column A indicates the name of the gene (polynucleotide); Column B indicates the disease(s) or disorder(s) associated with the gene (polynucleotide); Column C-D indicates FMRP targets in hPSCs (C) and neurons (D): a gene (polynucleotide) name in these columns indicates that it was identified as an FMRP target by eCLIP-seq in that cell type (e.g., hPSC or neuron). “#N/A” indicates that the given gene (polynucleotide) was not identified as such. Columns E-K present RNAseq data from FXS hPSCs (E), FXS neurons (F), DS hPSCs (G), DS neurons (H), DS FMRP CRISPRa 48 hr timepoint (I), DS FMRP CRISPRa 120 hr timepoint (J) and DS CRISPRa post-treatment timepoint (K). A value in these aforementioned columns indicates that the gene (polynucleotide) was significantly differentially expressed by RNAseq in a given dataset. Values=log2FoldChange; #N/A indicates that the given gene (polynucleotide) was not significantly differentially expressed by RNAseq. Column L-N present upstream regulator analyses from DS FMRP CRISPRa (L), FXS (M) and DS (N) RNAseq datasets, including all timepoints and cell types. A gene name in Columns L-N indicates that the gene was detected as being an upstream regulator in the RNAseq datasets, predicted to mediate the downstream changes in gene expression. A positive (solid line cell) v alue (z-score) means the function of that gene is predicted to be activated in a dataset, and a negative (dotted line cell) value (z-score) means the function of that gene is predicted to be inhibited in a dataset.

By way of a nonlimiting, representative example, KDM1A was discovered to be an FMRP target in hPSCs (column C), upregulated at the transcript level in DS hPSCs (column G) and identified as an upstream regulator mediating gene expression changes in all datasets (columns L-N). KDM1A function is expected or predicted to be activated in FXS and DS and inhibited by FMRP upregulation. Accordingly, KDM1A function is aberrantly activated in both FXS and DS, and FMRP upregulation is able to reverse such an upregulation, increase in expression, expression level, function or activation. KDM1A is also directly bound by FMRP, which is the predicted mechanism of action.

In some cases, genes (polynucleotides) in the datasets were selected because they were also identified in other neurodevelopmental disease datasets (e.g., exome sequencing from autism, developmental delay). The methodological details for each dataset (e.g., RNAseq, eCLIPseq) are described in Example 8 hereinbelow.

### Example 8: Materials and Methods

The materials and methods described herein relate to the above Examples and the results obtained from the described experiments, studies and analyses.

Stem cell resources and culture. The XY human embryonic stem cell line H1 was commercially obtained from WiCell Research Institute and was used to generate isogenic FMR1^{y/+} and FMR1^{y/-} cell lines (S. G. Susco et al., Dev Biol 468, 93-100 (2020)), as well as FXR1^{-/-} cell lines using CRISPR-Cas9. The XY human DS patient iPSC lines UWWC1-DS1, UVVWC1-2DS3 and the euploid control UWWC1-DS2U (isogenic with UWWC1-DS1) were commercially obtained from WiCell Research Institute (J. P. Weick et al., Proceedings of the National Academy of Sciences of the United States of America 110, 9962-9967 (2013)), referred to as DS iPSC A, DS iPSC B and control iPSC B, respectively, in the studies described herein. The control iPSC line CW60278, referred to as control iPSC A, was obtained from the California Institute for Regenerative Medicine iPSC repository (Fujifilm, Cellular Dynamics). Three FXS patient iPSCs were reprogrammed at the Harvard Stem Cell Institute Core (Cambridge MA) with Sendai virus using XY patient fibroblasts. The following fibroblast cell lines were obtained from the NIGMS Human Genetic Cell Repository at the Coriell Institute for Medical Research: GM05131 GM04026 and GM09497, referred to as FXS iPSC A, FXS iPSC B and FXS iPSC C in the described study, respectively, after reprogramming. XY cell lines were selected based on clinical data indicating that males are typically more severely affected by FXS than females and to avoid heterogeneity with respect to X-chromosome inactivation in edited clones. All studies using hESCs/iPSCs followed institutional IRB and ESCRO guidelines approved by Harvard University. Cell culture was carried out as previously described (Hazelbaker, D. Z. et al., Stem cell reports 9, 1315-1327 (2017); Hazelbaker, D. Z. et al., bioRxiv (2019); Bara, A. M. et al., Stem Cell Res 17, 441-443 (2016)). In brief, stem cells were grown and maintained in mTeSR medium (Stem Cell Technologies) on geltrex-coated (Life Technologies) plates. All cell lines underwent QC testing to confirm expected karyotypes and genotypes, absence of mycoplasma, expression of pluripotency markers and tri-lineage potential. SNP genotyping was performed using the Infinium PsychArray (Illumina) and chromosomal alterations were evaluated using the MoChA caller (Mattioli, F. et al., bioRxiv (2019)). G-band karyotyping analysis was performed by Cell Line Genetics. Replicates for experiments using hPSCs refer to separate wells or plates of a given hPSC line.

CRISPR-Cas9 based genome engineering. CRISPR-Cas9 based genome engineering experiments were carried out as previously described (Hazelbaker, D. Z. et al., Stem cell reports 9, 1315-1327 (2017); Hazelbaker, D. Z. et al., bioRxiv (2019); Bara, A. M. et al., Stem Cell Res 17, 441-443 (2016)). In brief, to generate FXR1^{-/-} cells, the hPSC line H1 was transfected with Cas9 nuclease plus an FXR1 gRNA targeting exon 8 of the full-length gene upstream of predicted functional domains (AGCTCAATGGCGGTAACTCC (SEQ ID NO: 7)), using the NEON system (Life Technologies) followed by clonal isolation and screening. To generate CRISPRa cell lines, TRE-dCas9-VPR- eGFP was inserted into the AAVS1 locus of the DS patient iPSC A (UWWC1-DS1) using TALENs, as previously described (Hazelbaker, D. Z. et al., Scientific reports 10, 635 (2020)). Three gRNAs targeting FMR1 for CRISPRa (g1: GCGCTGCTGGGAACCGGCCG (SEQ ID NO: 8), g2: CAGGTCGCACTGCCTCGCGA (SEQ ID NO: 9), g3: AGACCAGACACCCCCTCCCG (SEQ ID NO: 10)) were designed with the CRISPR-ERA tool (Liu, H. et al. Bioinformatics 31, 3676-3678 (2015), cloned into a multiplexed piggyBac vector and co-transfected in the presence of a piggyBac transposase, as previously described (Hazelbaker, D. Z. et al., Scientific reports 10, 635 (2020)). Following selection with G418 and blasticidin, cells were assessed for EGFP+/mRFP+ fluorescence and FMRP expression following doxycycline induction.

Generation of human excitatory neurons. Human neurons were generated as previously described (Zhang, Y. et al., Neuron 78, 785-798 (2013); Nehme, R. et al., Cell reports 23, 2509-2523 (2018)). In brief, hPSCs were transduced with TetO-Ngn2-T2A-Puro and Ubiq-rtTA lentivirus or TetO-Ngn2-P2A-Zeo and CAG-rtTA were integrated into the AAVS1 safe-harbor locus using TALENs. Cells were then treated with doxycycline to induce ectopic Ngn2 expression combined with the extrinsic addition of SMAD inhibitors (SB431542, 1614, Tocris, and LDN-193189, 04-0074, Stemgent), Wnt inhibitors (XAV939, 04-00046, Stemgent) and neurotrophins (BDNF, GDNF, CNTF) followed by puromycin treatment to eliminate uninfected stem cells and maintenance in Neurobasal medium. Ultra-high lentiviral titer was generated by Alstem, LLC. Alternatively, TetO-Ngn2-P2A-Zeo and CAG-rtTA were integrated into the AAVS1 safe-harbor locus using TALENs. Replicates for experiments using neurons refer to independent neuronal differentiations from a given hPSC line.

eCLIP-seq and analyses. eCLIP was performed as previously described (Van Nostrand, E. L. et al., Nature methods 13, 508-514 (2016)). In brief, FMR1^{y/+} and FMR1^{y/-} hPSCs or FMR1^{y/+} and FMR1^{y/-} neurons at day 14 of in vitro differentiation were UV-crosslinked, lysed, sonicated and treated with RNAse I. 2% of each lysate sample was used to generate a parallel size-matched input (SMInput) library. The remaining lysates were used for immunoprecipitation (IP) with the anti-FMRP antibodies RN016P (MBL International) and ab17722 (Abcam). Bound RNA fragments in the IPs were dephosphorylated and 3′-end ligated to an RNA adapter. Complexes from SMlnputs and IPs were run on an SDS-PAGE gel and transferred to a nitrocellulose membrane. Membranes were excised, and proteinase K treated to release RNA from protein complexes. Samples were then dephosphorylated and 3′-end ligated to an RNA adaptor. All IP and SMlnput samples were reverse transcribed using AffinityScript (Agilent). cDNAs were 5′-end ligated to a DNA adaptor. cDNA yields were quantified by qPCR, and 100-500 fmol of libraries were generated using Q5 PCR mix (New England Biolabs). Reads were trimmed to remove adapters, and mapped to human repetitive elements from RepBase (version 18.05) by STAR. The remaining reads that were not mapped to repetitive elements were mapped to the human genome assembly hg19 by STAR. PCR duplicates were removed using the unique molecular identifier (UMI) sequences. Peaks were assigned by CLIPper, and annotated to gene regions in Gencode (v19) with the following priority order: CDS, 5′UTR, 3′ UTR, proximal intron, and distal intron. Distal intron regions were defined as the intronic regions more than 500 bp from an exon-intron junction(distintron500), and proximal intron regions were defined as the intronic regions up to 500 bp from an exon-intron junction (proxintron500). The peak fold changes were calculated by normalizing usable reads between immunoprecipitation and the SMInput with a peak threshold of at least 8-fold enrichment in IP over the SMInput and p-values ≤10⁻³. Enriched p-values were calculated by Chi-square test, or Fisher's exact test if the usable read number in IP or SMlnput was below 5. The above eCLIP-seq analysis pipeline was also performed using FMR1^{y/-} hPSCs (RN016P) and FMR1^{y/-} neurons (RN016P and ab17722). Significant peaks in the FMR1^{y/+} dataset were removed from analysis if an overlapping significant peak also occurred in the corresponding FMR1^{y/-} dataset.

### Gene Ontology (GO) Analyses

GO analyses of eCLIP-seq data were performed using the Panther overrepresentation test and GO database annotation with Fisher test and Bonferroni correction. To determine the size of the RNA universe in both hPSCs and neurons, the TPM counts from the RNA-seq data were assessed. A gene was counted as expressed if it had an average TPM≥1 across five replicates in each FMR1^{y/+} cell type. This generated 15,316 RNAs expressed in neurons, and 14,233 RNAs expressed in hPSCs. These universes were then used to establish the background for GO analysis.

RNA motif and structure analyses. To analyze potential motif enrichment in FMRP binding sites, HOMER analyses were processed as described in CLIP_analysis_legacy (https://github.com/YeoLab/clip_analysis_legacy). Briefly, HOMER was used to identify de novo motifs by comparing significant enriched peaks with randomly defined peaks. The command was ‘findMotifs.p1 <foreground>hg19<output location>-ma -S 20 -len 6 -p 4 -bg<background>’. Foreground was a bed file of significant enriched peaks; the background was randomly defined peaks within the same annotated region as foreground peaks. To analyze potential structure enrichment in FMRP binding sites, base pairing probabilities were calculated in and around FMRP binding sites detected by eCLIP-seq. FMRP binding sites were split into length categories of ≤50, or ≤200. Binding sites in each category were extended symmetrically in size to encompass either exactly 50 bases, or exactly 200 bases. The equally sized portions of sequence then underwent computational structure prediction with RNAfold 2.0, using the command “RNAfold -p” to calculate pair probabilities of every base in the sequence. Pair probabilities were summed for each base to give the final probability of a base being paired. Base pairing probabilities were also predicted for equally sized regions flanking the 50-base or 200-base FMRP binding sites. Base pairing probabilities were averaged for bases in flanking or FMRP region, which were each divided in two. Averages were plotted as violin plots with Seaborn 0.9.0.

RNA-seq of FXS and DS Cell Lines. RNA was extracted from hPSCs and neurons using the AllPrep DNA/RNA/miRNA Universal Kit (Qiagen) using five biological replicates for each genotype and cell type (e.g., five separate wells of hPSCs or five independent batches of neuronal differentiations). Sequencing libraries were prepared using the Illumina TruSeq HS Stranded Total RNA kit with Ribo-Zero Gold for rRNA depletion and quantified using the Agilent Bioanalyzer RNA Pico kit. Libraries were sequenced on a HiSeq 2500 at the Broad Institute Genomics Platform to generate 100 bp paired end reads. RNA-seq QC and analysis was performed by the Harvard Chan Bioinformatics Core, Harvard T.H. Chan School of Public Health, Boston, MA. Reads were processed to counts through the bcbio RNA-seq pipeline implemented in the bcbio-nextgen project (https://bcbio-nextgen.readthedocs.org/en/latest/). Raw reads were examined for quality issues using FastQC (http://www.bioinformatics.babraham.ac.uk/projects/fastqc/) to ensure library generation and sequencing were suitable for further analysis. As necessary, adapter sequences, other contaminant sequences, such as polyA tails and low quality sequences with PHRED quality scores less than five, were trimmed from reads using cutadapt (M. Martin, Cutadapt removes adapter sequences from high-throughput sequencing reads. 17, 3 (2011)). Trimmed reads were aligned to Ensembl build GRCh38 90 of the Homo sapiens genome (human), using STAR (A. Dobin et al., STAR: ultrafast universal RNA-seq aligner. Bioinformatics (Oxford, England) 29, 15-21 (2013)). Alignments were checked for evenness of coverage, rRNA content, genomic context of alignments (for example, alignments in known transcripts and introns), complexity and other quality checks using a combination of FastQC, Qualimap (F. Garcia-Alcalde et al., Qualimap: evaluating next-generation sequencing alignment data. Bioinformatics 28, 2678-2679 (2012)), MultiQC (https://github.com/ewels/MultiQC) and custom tools. Counts of reads aligning to known genes were generated by featureCounts (Y. Liao, G. K. Smyth, W. Shi, featureCounts: an efficient general purpose program for assigning sequence reads to genomic features. Bioinformatics 30, 923-930 (2014)). In parallel, TPM measurements per isoform were generated by quasialignment using Salmon (R. Patro, G. Duggal, C. Kingsford, Accurate, fast, and model-aware transcript expression quantification with Salmon. bioRxiv, (2015)). Differential expression at the gene level was called with DESeq2 (M. I. Love, W. Huber, S. Anders, Moderated estimation of fold change and dispersion for RNA-seq data with DESeq2. Genome Biol 15, 550 (2014)), preferring to use counts per gene estimated from the Salmon quasialignments by tximport (C. Soneson, M. I. Love, M. D. Robinson, Differential analyses for RNA-seq: transcript-level estimates improve gene-level inferences. F1000Res 4, 1521 (2015)). Quantifying at the isoform level has been shown to produce more accurate results at the gene level.

### RNA-seq of CRISPRa Cell Lines

RNA was extracted from hPSCs using the AllPrep DNA/RNA/miRNA Universal Kit (Qiagen) using four replicates (e.g., four separate wells of hPSCs) for each condition: Euploid Control iPSC B, DS CRISPRa (untreated), DS 48 hr FMRP CRISPRa, DS 120 hr FMRP CRISPRa and DS 120 hr on/120 hr off FMRP CRISPRa. Libraries were prepared using Roche Kapa mRNA HyperPrep strand specific sample preparation kits from 200 ng of purified total RNA according to the manufacturer's protocol on a Beckman Coulter Biomek i7. The finished dsDNA libraries were quantified by Qubit fluorometer and Agilent TapeStation 4200. Uniquely dual indexed libraries were pooled in equimolar ratio and shallowly sequenced on an Illumina MiSeq to further evaluate library quality and pooling balance. The final pool was sequenced on an Illumina NovaSeq 6000 targeting 30 million 100 bp read pairs per library. Sequenced reads were aligned to the UCSC hg19 reference genome assembly and gene counts were quantified using STAR (v2.7.3a) (A. Dobin et al., STAR: ultrafast universal RNA-seq aligner. Bioinformatics (Oxford, England) 29, 15-21 (2013)). Differential gene expression testing was performed by DESeq2 (v1.22.1) (M. I. Love, W. Huber, S. Anders, Moderated estimation of fold change and dispersion for RNA-seq data with DESeq2. Genome Biol 15, 550 (2014)). RNAseq analysis was performed using the VIPER snakemake pipeline (M. Cornwell et al., VIPER: Visualization Pipeline for RNA-seq, a Snakemake workflow for efficient and complete RNA-seq analysis. BMC Bioinformatics 19, 135 (2018)). Library preparation, Illumina sequencing and VIPER workflow were performed by the Dana-Farber Cancer Institute Molecular Biology Core Facilities and we appreciate the analytical support from Zach Herbert. Ingenuity Pathway Analysis (IPA) was used for analyses of upstream regulators.

Western blot analysis. Cells were lysed using RIPA lysis buffer (Life Technologies) with protease inhibitors (cOmplete, Mini, EDTA-free Protease Inhibitor Cocktail, Roche). 20 μg of protein as determined by Peirce BCA Protein Assay kit (Thermo Scientific) was loaded onto Bolt 4-12% Bis-Tris Plus gels (Invitrogen), transferred using the iBlot2 system (Thermo Scientific), blocked in 5% milk in TBST, and then incubated with primary antibodies in 1% milk in TBST overnight at 4° C. Membranes were rinsed in TBST, incubated with secondary antibodies for 1 hour at room temperature, rinsed in TBST, and then developed using SuperSignal West Femto Maximum Sensitivity Substrate (Thermo Scientific). The following primary antibodies were used: anti-FMRP (Abcam ab17722), anti-GAPDH (EMD MAB374), anti-ADARB1 (Thermo Scientific PAS-34828), anti-NCAM2 (Abcam ab173297), anti-DYRK1A (Bethyl A303-802A), anti-FXR1P (ML13 courtesy E. Khandjian), anti-PCP4 (Abcam ab197377), anti-CBS (Proteintech 14787-1-AP), anti-APP (Abcam ab32136), anti-BACE2 (Abcam ab270458). For quantification, bands were analyzed in FIJI, normalized to GAPDH, averaged, and plotted with SEM for error bars. All Western blots were performed on triplicate samples and significance was calculated by unpaired two-tailed t-test for comparisons between two groups. Prism (GraphPad Software) was used for statistical analyses.

IP-Western Blot. For FMRP IP-Western blot analysis, neurons were lysed in Pierce IP lysis buffer (Life Technologies) and quantified with Peirce BCA Protein Assay kit (Thermo Scientific). 1 mg of protein lysate was incubated with 1 μg of antibody overnight with rotating at 4° C. Protein G magnetic beads (Pierce) were washed twice in cold lysis buffer and added at a concentration of 0.437 mg of beads to 1 μg of antibody per IP and incubated at 4° C. with rotating for 4 hours. Samples were placed on the Dynamag2 (Invitrogen), and the supernatant sample was collected. Samples were then washed once with cold lysis buffer (wash 1), and twice with cold PBS (washes 2 and 3). The washed beads were then resuspended, and equal fractions were boiled in 4× Laemmli buffer with BME for 5 minutes, placed on the magnet, and loaded into Bolt 4-12% Bis-Tris Plus gels (Invitrogen) for Western blotting as described above. FMR1^{y/+} neurons were immunoprecipitated with anti-FMRP (Abcam ab17722) and anti-IgG (Sigma 12370) and FMR1^{y/-} neurons were immunoprecipitated with anti-FMRP (Abcam ab17722). All three immunoprecipitations were blotted for FXR1P with anti-FXR1P ML13 (courtesy E. Khandjian).

ENCODE Dataset Meta-analyses. To analyze data across chromosomes for 120 RNA binding protein (RBPs) from K562 cells collected with eCLIP-seq as part of the ENCODE Consortium Project, the number of replicable binding sites was counted on each chromosome and normalized to the number of transcribed and mappable bases on the given chromosome (based on Hg19 NCBI transcript annotations and ENCODE blacklist data). Counts were also normalized by the total number of replicable binding sites for a given RBP, and scaled by a constant for viewing. Heat maps were plotted and RBPs hierarchically clustered using Seaborn 0.9.0.

### Comparative Analyses

To compare the human targets with published datasets, 842 mouse FMRP targets previously identified by Darnell et al., (J. C. Darnell et al., Cell 146, 247-261 (2011)) were utilized; the corresponding 865 human homologs were identified using Ensembl Biomart. The ‘high’ and ‘stringent’ mouse FMRP CA1 targets from Sawicka et al., (K. Sawicka et al., Elife 8, (2019)) were also used, resulting in 1266 targets after converting from mouse to human homologs using Ensembl Biomart. 3322 targets were identified from a K562 FMRP eCLIP-seq dataset from the ENCODE project. For epilepsy comparisons, the list of top 200 genes with burden of deleterious ultra-rare variants with an allele count ≤3 in all epilepsy cases (Epi25, 2019) were used. For statistical tests of enrichment and overlap, the hypergeometric test for over enrichment with Bonferroni correction was used. To determine the size of the RNA universe in both hPSCs and neurons, the TPM counts from the RNA-seq data were assessed, and a gene was counted as expressed if it had an average TPM≥1 across five replicates in each FMR1^{y/+} cell type. This generated 15,316 RNAs expressed in neurons, and 14,233 RNAs expressed in hPSCs. These universes were then applied to the developmental disorders, autism, epilepsies and HSA21 gene lists to establish expression. The Allen BrainSpan Atlas of the developing human brain data was used to establish expression in the fetal brain (https://www.brainspan.org/static/home downloaded Dec. 12, 2019). Only prenatal time points were included; all brain regions were included. A gene was considered expressed if the RPKM was ≥1.

Statistical analysis. Replicates for experiments using hPSCs refer to separate wells or plates, and replicates for experiments using neurons refer to independent neuronal differentiations. For eCLIP-seq experiments, peak fold changes were calculated by normalizing usable reads between IP and the SMlnput, with a peak threshold of at least 8-fold enrichment in IP over the SMlnput and p-values ≤10⁻³. Enriched p-values were calculated by Chi-square test or Fisher's exact test if the usable read number in IP or SMlnput was below 5. For RNA-seq analyses, four to five replicates per genotype and cell type and an adjusted p-value cutoff of 0.05 were used. For mRNA-seq of CRISPRa lines, a log2foldchange cutoff of over 1 or under −1 was also applied. For Western blot analyses, experiments were performed on triplicate samples and significance was calculated by unpaired two-tailed t-test for comparisons between two groups; Prism (GraphPad Software) was used for statistical analyses. For statistical tests of enrichment and overlap, the hypergeometric test for over enrichment with Bonferroni correction for multiple comparison testing was used. P values (or adjusted P values, where applicable) ≤0.05 were considered statistically significant.

### Other Embodiments

Additional embodiments of the invention are described below.
Embodiment 1: A method of reducing the level of a Dual Specificity Tyrosine Phosphorylation Regulated Kinase 1A (DYRK1A) and/or an amyloid-beta precursor (APP) polypeptide or a polynucleotide encoding such polypeptide in a cell, the method comprising contacting the cell with an expression vector comprising a polynucleotide sequence encoding a Fragile X mental retardation protein (FMRP) polypeptide or a fragment thereof, thereby decreasing the level of the DYRK1A and/or APP protein or polynucleotide in the cell.
Embodiment 2: The method of embodiment 1, wherein the cell comprises an increased level of DYRK1A and/or APP.
Embodiment 3: The method of embodiment 2, wherein the level of DYRK1A and/or APP is increased by at least about 10% relative to a reference.
Embodiment 4: The method of embodiment 2 or 3, wherein the increased level of DYRK1A and/or APP is associated with a neurodevelopmental disorder or neurodegenerative disorder.
Embodiment 5: The method of embodiment 4, wherein the neurodevelopmental disorder is autism, Fragile X syndrome, or Down syndrome, or, optionally, a disease or disorder associated with Down syndrome.
Embodiment 6: The method of embodiment 4, wherein the neurodegenerative disorder is Alzheimer's disease.
Embodiment 7: The method of any one of embodiments 1-6, wherein the expression vector is a lentiviral vector, adenoviral vector, or adeno-associated viral vector.
Embodiment 8: The method of any one of embodiments 1-7, wherein the cell is a mammalian cell.
Embodiment 9: The method of any one of embodiments 1-8, wherein the cell is in vitro, in vivo, or ex vivo.
Embodiment 10: A method for treating a disease associated with an increase in a DYRK1A and/or APP polypeptide in a subject, the method comprising administering an effective amount of a Fragile X mental retardation protein (FMRP) polypeptide or fragment thereof or a polynucleotide sequence encoding said polypeptide or a fragment thereof to the subject.
Embodiment 11: The method of embodiment 10, wherein the level of DYRK1A and/or APP is increased by at least about 10% relative to a reference.
Embodiment 12: The method of embodiment 10 or 11, wherein the increased level of DYRK1A and/or APP is associated with a neurodevelopmental disorder or neurodegenerative disorder.
Embodiment 13: The method of embodiment 12, wherein the neurodevelopmental disorder is autism, Fragile X syndrome, or Down syndrome, or optionally, a disease or disorder associated with Down syndrome.
Embodiment 14: The method of embodiment 12, wherein the neurodegenerative disorder is Alzheimer's disease.
Embodiment 15: The method of any one of embodiments 10-14, wherein the subject is a mammal.
Embodiment 16: The method of embodiment 15, wherein the mammal is a rodent, canine, feline, or human.
Embodiment 17: The method of any one of embodiments 10-16, wherein the polynucleotide is present in an expression vector.
Embodiment 18: The method of embodiment 17, wherein the expression vector is a lentiviral vector, adenoviral vector, or adeno-associated viral vector.
Embodiment 19: A method of treating a subject having or having a propensity to develop Alzheimer's disease, the method comprising administering to the subject an expression vector comprising a polynucleotide sequence encoding a Fragile X mental retardation protein (FMRP) polypeptide or a fragment thereof, thereby treating Alzheimer's disease.
Embodiment 20: The method of embodiment 19, wherein Alzheimer's disease is associated with at least about a 10% increase in the level of APP in a cell of the subject relative to the level of APP present in a corresponding cell of a control subject.
Embodiment 21: The method of embodiment 19 or 20, wherein the subject has Down syndrome, or, optionally, a disease or disorder associated with Down syndrome.
Embodiment 22: A method of treating a subject having or having a propensity to develop Fragile X syndrome, the method comprising administering to the subject an effective amount of a Fragile X mental retardation protein (FMRP) polypeptide or a fragment thereof or a polynucleotide sequence encoding FMRP or a fragment thereof.
Embodiment 23: The method of embodiment 22, wherein the level of DYRK1A and/or APP in a cell of the subject is increased by at least about 10% relative to the level present in a corresponding cell of a control subject that does not have Fragile X syndrome.
Embodiment 24: The method of any one of embodiments 19-23, wherein the subject is a mammal.
Embodiment 25: The method of embodiment 24, wherein the mammal is a rodent, canine, feline, or human.
Embodiment 26: The method of embodiment 25, wherein the mammal is a human.
Embodiment 27: The method of any one of embodiments 19-26, wherein the polynucleotide is present in an expression vector.
Embodiment 28: The method of embodiment 27, wherein the expression vector is a lentiviral vector, adenoviral vector, or adeno-associated viral vector.
Embodiment 29: The method of any one of embodiments 5, 13, or 21, wherein the disease or disorder associated with Down syndrome is autism, a seizure disorder, or a leukemia.

What is claimed is:

1. A method of modulating the expression, expression level, amount, or activity of a gene as set forth in Table 5, or a polypeptide encoded by such gene, in a cell, the method comprising: contacting the cell with an expression vector comprising a polynucleotide sequence encoding a Fragile X mental retardation protein (FMRP) polypeptide or a fragment thereof, thereby modulating the expression, expression level, amount, or activity of the gene as set forth in Table 5, or the encoded polypeptide, in the cell.

2. The method of claim 1, wherein the modulating comprises an increase or decrease in expression or activity of the gene or the encoded polypeptide.

3. The method of claim 1, wherein the gene is associated with Fragile X syndrome (FXS) and/or Down syndrome (DS).

4. The method of claim 1, wherein the modulation is by at least 20% relative to a control cell.

5. A method of modulating the expression or activity of a gene set forth in Table 5, or the encoded product of the gene, which is associated with a neurodevelopmental, or neurodegenerative disease or disorder, the method comprising: administering to the subject an effective amount of a Fragile X mental retardation protein (FMRP) polypeptide, or a fragment thereof, or a polynucleotide sequence encoding FMRP, or a fragment thereof, to modulate the expression, expression level, amount, or activity of the gene, or the encoded product of the gene.

6. The method of claim 5, wherein the expression or activity of the gene, or the encoded gene product, is increased or decreased in the subject following the administration of FMRP to the subject.

7. The method of claim 5, wherein the developmental, neurodevelopmental, or neurodegenerative disease or disorder is Fragile X syndrome (FXS) and/or Down syndrome (DS).

8. The method of claim 5, wherein the subject is at risk of having, or has a propensity to develop one or more neurodevelopmental or neurodegenerative diseases or disorders.

9. The method of claim 8, wherein the neurodevelopmental or neurodegenerative disease or disorder is Fragile X syndrome (FXS) and/or Down syndrome (DS).

10. The method of claim 5, wherein the polynucleotide sequence encoding FMRP is present in an expression vector.

11. The method of claim 10, wherein the expression vector is a lentiviral vector, adenoviral vector, or adeno-associated viral vector.

12. The method of claim 5, wherein the expression or activity of the gene or the encoded product of the gene is increased or decreased in the subject by at least 20% following administration of FMRP.

13. A method of treating or reducing the severity of a neurodevelopmental or a neurodegenerative disease or disorder, or the symptoms thereof in a subject, the method comprising: modulating the expression or activity of one or more genes set forth in Table 5 in a cell and/or in a subject.

14. The method of claim 13, wherein the neurodevelopmental or neurodegenerative disease or disorder is Fragile X syndrome (FXS) and/or Down syndrome (DS).

15. A method of treating Fragile X syndrome (FXS) and/or Down syndrome (DS), or the symptoms thereof, in a subject the method comprising: modulating the expression or activity of one or more genes set forth in Table 5 in the subject.

16. The method of claim 15, wherein the method comprises administering to the subject an effective amount of an agent that modulates the expression or activity of one or more genes set forth in Table 5 in the subject.

17. The method of claim 16, wherein the agent is a polypeptide, a polynucleotide, a drug, or a small molecule compound.

18. The method of claim 16, wherein the agent is a Fragile X mental retardation protein (FMRP) polypeptide, or a fragment thereof, or a polynucleotide sequence encoding FMRP, or a fragment thereof.

19. A panel comprising a plurality of the genes of Table 5.

20. The panel of claim 19, wherein Fragile X mental retardation protein (FMRP) polypeptide, or a fragment thereof, modulates the expression level or activity of the genes.


SEQUENCE LISTING (10 sequences)

SEQ ID NO: 1 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 2 - DNA, 2378 bp, Homo sapiens
  source 1..2378: mol_type = other DNA
agagcaaggacgcggcggatcccactcgcacagcagcgcactcggtgccccgcgcagggtcgcagatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaactccctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 3 - protein, 763 aa, Homo sapiens
MHTGGETSACKPSSVRLAPSFSFHAAGLQMAGQMPHSHQYSDRRQPNISDQQVSALSYSDQIQQPLTNQVMPDIVMLQRRMPQTFRDPATAPLRKLSVDLIKTYKHINEVYYAKKKRRHQQGQGDDSSHKKERKVYNDGYDDDNYDYIVKNGEKWMDRYEIDSLIGKGSFGQVVKAYDRVEQEWVAIKIIKNKKAFLNQAQIEVRLLELMNKHDTEMKYYIVHLKRHFMFRNHLCLVFEMLSYNLYDLLRNTNFRGVSLNLTRKFAQQMCTALLFLATPELSIIHCDLKPENILLCNPKRSAIKIVDFGSSCQLGQRIYQYIQSRFYRSPEVLLGMPYDLAIDMWSLGCILVEMHTGEPLFSGANEVDQMNKIVEVLGIPPAHILDQAPKARKFFEKLPDGTWNLKKTKDGKREYKPPGTRKLHNILGVETGGPGGRRAGESGHTVADYLKFKDLILRMLDYDPKTRIQPYYALQHSFFKKTADEGTNTSNSVSTSPAMEQSQSSGTTSSTSSSSGGSSGTSNSGRARSDPTHQHRHSGGHFTAAVQAMDCETHSPQVRQQFPAPLGWSGTEAPTQVTVETHPVQETTFHVAPQQNALHHHHGNSSHHHHHHHHHHHHHGQQALGNRTRPRVYNSPTNSSSTQDSMEVGHSHHSMTSLSSSTTSSSTSSSSTGNQGNQAYQNRPVAANTLDFGQNGAMDVNLTVYSNPRQETGIAGHPTYQFSANTGPAHYMTEGHLTMRQGADREESPMTGVCVQQSPVASS

SEQ ID NO: 4 - DNA, 2422 bp, Homo sapiens
  source 1..2422: mol_type = other DNA
gttatagttttgccgctggactcttccctcccttcccccaccccatcaggatgatatgagacttgaaagaagacgatgcatacaggaggagagacttcagcatgcaaaccttcatctgttcggcttgcaccgtcattttcattccatgctgctggccttcagatggctggacagatgccccattcacatcagtacagtgaccgtcgccagccaaacataagtgaccaacaggtttctgccttatcatattctgaccagattcagcaacctctaactaaccaggtgatgcctgatattgtcatgttacagaggcggatgccccaaaccttccgtgacccagcaactgctcccctgagaaaactttctgttgacttgatcaaaacatacaagcatattaatgaggtttactatgcaaaaaagaagcgaagacaccaacagggccagggagacgattctagtcataagaaggaacggaaggtttacaatgatggttatgatgatgataactatgattatattgtaaaaaacggagaaaagtggatggatcgttacgaaattgactccttgataggcaaaggttcctttggacaggttgtaaaggcatatgatcgtgtggagcaagaatgggttgccattaaaataataaagaacaagaaggcttttctgaatcaagcacagatagaagtgcgacttcttgagctcatgaacaaacatgacactgaaatgaaatactacatagtgcatttgaaacgccactttatgtttcgaaaccatctctgtttagtttttgaaatgctgtcctacaacctctatgacttgctgagaaacaccaatttccgaggggtctctttgaacctaacacgaaagtttgcgcaacagatgtgcactgcactgcttttccttgcgactccagaacttagtatcattcactgtgatctaaaacctgaaaatatccttctttgtaaccccaaacgcagtgcaatcaagatagttgactttggcagttcttgtcagttggggcagaggatataccagtatattcagagtcgcttttatcggtctccagaggtgctactgggaatgccttatgaccttgccattgatatgtggtccctcgggtgtattttggttgaaatgcacactggagaacctctgttcagtggtgccaatgaggtagatcagatgaataaaatagtggaagttctgggtattccacctgctcatattcttgaccaagcaccaaaagcaagaaagttctttgagaagttgccagatggcacttggaacttaaagaagaccaaagatggaaaacgggagtacaaaccaccaggaacccgtaaacttcataacattcttggagtggaaacaggaggacctggtgggcgacgtgctggggagtcaggtcatacggtcgctgactacttgaagttcaaagacctcattttaaggatgcttgattatgaccccaaaactcgaattcaaccttattatgctctgcagcacagtttcttcaagaaaacagctgatgaaggtacaaatacaagtaatagtgtatctacaagccccgccatggagcagtctcagtcttcgggcaccacctccagtacatcgtcaagctcaggtggctcatcggggacaagcaacagtgggagagcccggtcggatccgacgcaccagcatcggcacagtggtgggcacttcacagctgccgtgcaggccatggactgcgagacacacagtccccaggtgcgtcagcaatttcctgctcctcttggttggtcaggcactgaagctcctacacaggtcactgttgaaactcatcctgttcaagaaacaacctttcatgtagcccctcaacagaatgcattgcatcatcaccatggtaacagttcccatcatcaccaccaccaccaccaccatcaccaccaccatggacaacaagccttgggtaaccggaccaggccaagggtctacaattctccaacgaatagctcctctacccaagattctatggaggttggccacagtcaccactccatgacatccctgtcttcctcaacgacttcttcctcgacatcttcctcctctactggtaaccaaggcaatcaggcctaccacaatcgcccagtggctgctaataccttggactttggacagaatggagctatggacgttaatttgaccgtctactccaatccccgccaagagactggcatagctggacatccaacataccaattttctgctaatacaggtcctgcacattacatgactgaaggacatctgacgatgaggcaaggggctgatagagaagagtcccccatgacaggagtttgtgtgcaacagagtcctgtagctagctcgtgactacattgaaacttgagtttgtttcttgtgtgtttttatagaagtggtgtttttt

SEQ ID NO: 5 - protein, 632 aa, Homo sapiens [sequence omitted]

SEQ ID NO: 6 - DNA, 4362 bp, Homo sapiens
  source 1..4362: mol_type = other DNA
acggcgagcgcgggcggcggcggtgacggaggcgccgctgccagggggcgtgcggcagcgcggcggcggcggcggcggcggcggcggcggaggcggcggcggcggcggcggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagagggtgttagtggcttcatcagttgtagcaggggaatcccagaaacctgaactcaaggcttggcagggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccctaaactgcataattctgaagttatatttcctataccatttccgtaattcttattccatattagaaaactttgttaggccaaagacaaatagtaggcaagatggcacagggcatgaaatgaacacaaattatgctaagaattttttattttttggtattggccataagcaacaattttcagatttgcacaaaaagataccttaaaatttgaaacattgcttttaaaactacttagcacttcagggcagattttagttttattttctaaagtactgagcagtgatattctttgttaatttggaccattttcctgcattgggtgatcattcaccagtacattctcagtttttcttaatatatagcatttatggtaatcatattagacttctgttttcaatctcgtatagaagtcttcatgaaatgctatgtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagcttttttttttaattttgtctgccccaagttttgtgaaatttttcatattttaatttcaagcttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgcacatgatttcacaaatattaaaaagtcttttaaaaagtattgccaaacattaatgttgatttctagttatttattctgggaatgtatagtatttgaaaacagaaattggtaccttgcacacatcatctgtaagctgtttggttttaaaatactgtagataattaaccaaggtagaatgaccttgtaatgtaactgctcttgggcaatattctctgtacatattagcgacaacagattggattttatgttgacatttgtttggttatagtgcaatatattttgtatgcaagcagtttcaataaagtttgatcttcctctgctaaattgatgttgatgcaatccttacaaatgattgcttttaaaattttaagctaggaaaagaaatctatagaaagtgttctgttacaaaatgtaactgttaccattggaaatttcacgtcataggaagttagcctttatctacccaactttcaagaaggttctttaataaagcgaaaactcaaccaaatggtacttttccacagtgtaccattaaaatatgcactagtctctttttacaaggctgtattcagcaagggcctaacttgcttaaagtgtaattactaacttctaaaactgtactttgattcacatgttttcaaatggagttggagttcattcatattacaatatttgtgtgctaaacgtgtatgtttttcagttcaaagtcatgatgtttttaaaatcttattaaagtttcaaaaatctgaagattgtttatctagatgtaaatttt

SEQ ID NO: 7 - DNA, 20 bp, synthetic construct
  misc_feature 1..20: note = Description of Artificial Sequence: Synthetic oligonucleotide
  source 1..20: mol_type = other DNA
agctcaatggcggtaactcc

SEQ ID NO: 8 - DNA, 20 bp, synthetic construct
  misc_feature 1..20: note = Description of Artificial Sequence: Synthetic oligonucleotide
  source 1..20: mol_type = other DNA
gcgctgctgggaaccggccg

SEQ ID NO: 9 - DNA, 20 bp, synthetic construct
  misc_feature 1..20: note = Description of Artificial Sequence: Synthetic oligonucleotide
  source 1..20: mol_type = other DNA
caggtcgcactgcctcgcga

SEQ ID NO: 10 - DNA, 20 bp, synthetic construct
  misc_feature 1..20: note = Description of Artificial Sequence: Synthetic oligonucleotide
  source 1..20: mol_type = other DNA
agaccagacaccccctcccg